SEQUENCE LISTING (175 sequences)

SEQ ID NO: 1 - DNA, 3141 bp, Artificial Sequence
  OGT Sense DNA sequence/Nucleotide Sequence
atggcgtcttccgtgggcaacgtggccgacagcacagaaccaacgaaacgtatgctttccttccaagggttagctgagttggcacatcgagaatatcaggcaggagattttgaggcagctgagagacactgcatgcagctctggagacaagagccagacaatactggtgtgcttttattactttcatctatacacttccagtgtcgaaggctggacagatctgctcactttagcactctggcaattaaacagaacccccttctggcagaagcttattcgaatttggggaatgtgtacaaggaaagagggcagttgcaggaggcaattgagcattatcgacatgcattgcgtctcaaacctgatttcatcgatggttatattaacctggcagccgccttggtagcagcgggtgacatggaaggggcagtacaagcttacgtctctgctcttcagtacaatcctgatttgtactgtgttcgcagtgacctggggaacctgctcaaagccctgggtcgcttggaagaagccaaggcatgttatttgaaagcaattgagacgcaaccgaactttgcagtagcttggagtaatcttggctgtgttttcaatgcacaaggggaaatttggcttgcaattcatcactttgaaaaggctgtcacccttgacccaaactttctggatgcttatatcaatttaggaaatgtcttgaaagaggcacgcatttttgacagagctgtggcagcttatcttcgtgccctaagtttgagtccaaatcacgcagtggtgcacggcaacctggcttgtgtatactatgagcaaggcctgatagatctggcaatagacacctacaggcgggctatcgaactacaaccacatttccctgatgcttactgcaacctagccaatgctctcaaagagaagggcagtgttgctgaagcagaagattgttataatacagctctccgtctgtgtcccacccatgcagactctctgaataacctagccaatatcaaacgagaacagggaaacattgaagaggcagttcgcttgtatcgtaaagcattagaagtcttcccagagtttgctgctgcccattcaaatttagcaagtgtactgcagcagcagggaaaactgcaggaagctctgatgcattataaggaggctattcgaatcagtcctacctttgctgatgcctactctaatatgggaaacactctaaaggagatgcaggatgttcagggagccttgcagtgttatacgcgtgccatccaaattaatcctgcatttgcagatgcacatagcaatctggcttccattcataaggattcagggaatattccagaagccatagcttcttaccgcacggctctgaaacttaagcctgattttcctgatgcttattgtaacttggctcattgcctgcagattgtctgtgattggacagactatgatgagcgaatgaagaagttggtcagtattgtggctgaccagttagagaagaataggttgccttctgtgcatcctcatcatagtatgctatatcctctttctcatggcttcaggaaggctattgctgagaggcacggcaacctgtgcttagataagattaatgttcttcataaaccaccatatgaacatccaaaagacttgaagctcagtgatggtcggctgcgtgtaggatatgtgagttccgactttgggaatcatcctacttctcaccttatgcagtctattccaggcatgcacaatcctgataaatttgaggtgttctgttatgccctgagcccagacgatggcacaaacttccgagtgaaggtgatggcagaagccaatcatttcattgatctttctcagattccatgcaatggaaaagcagctgatcgcatccatcaggatggaattcatatccttgtaaatatgaatggctatactaagggcgctcgaaatgagctttttgctctcaggccagctcctattcaggcaatgtggctgggataccctgggacgagtggtgcgcttttcatggattatattatcactgatcaggaaacttcgccagctgaagttgctgagcagtattccgagaaattggcttatatgccccacactttttttattggtgatcatgctaatatgttccctcacctgaagaaaaaagcagtcatcgattttaagtccaatgggcacatttatgacaatcggatagttctgaatggcatcgacctcaaagcatttcttgatagtctaccagatgtgaaaattgtcaagatgaagtgtcctgatggaggagacaatgcagatagcagtaacacagctcttaatatgcctgttattcctatgaatactattgcagaagcagttattgaaatgattaaccgaggacagattcaaataacaattaatggattcagtattagcaatggactggcaactactcagatcaacaataaggctgcaactggagaggaggttccccgtaccattattgtaaccacccgttctcagtacgggttaccagaagatgccatcgtatactgtaactttaatcagttgtataaaattgacccttctactttgcagatgtgggcaaacattctgaagcgtgttcccaatagtgtactctggctgttgcgttttccagcagtaggagaacctaatattcaacagtatgcacaaaacatgggcctgccccagaaccgtatcattttttcacctgttgctcctaaagaggaacacgtcaggagaggccagctggctgatgtctgcttggacactccactctgtaatgggcacaccacagggatggatgtcctctgggcagggacccccatggtgactatgccaggagagactcttgcttctcgagttgcagcatcccagctcacttgcttaggttgtcttgagcttattgctaaaaacagacaagaatatgaagacatagctgtgaagctgggaactgatctagaatacctgaagaaagttcgtggcaaagtctggaagcaaagaatatctagccctctgttcaacaccaaacaatacacaatggaactagagcggctctatctacagatgtgggagcattatgcagctggcaacaaacctgaccacatgattaagcctgttgaagtcactgagtcagcataa

SEQ ID NO: 2 - protein, 1046 aa, Artificial Sequence
  OGT Polypeptide Sequence
MASSVGNVADSTEPTKRMLSFQGLAELAHREYQAGDFEAAERHCMQLWRQEPDNTGVLLLLSSIHFQCRRLDRSAHFSTLAIKQNPLLAEAYSNLGNVYKERGQLQEAIEHYRHALRLKPDFIDGYINLAAALVAAGDMEGAVQAYVSALQYNPDLYCVRSDLGNLLKALGRLEEAKACYLKAIETQPNFAVAWSNLGCVFNAQGEIWLAIHHFEKAVTLDPNFLDAYINLGNVLKEARIFDRAVAAYLRALSLSPNHAVVHGNLACVYYEQGLIDLAIDTYRRAIELQPHFPDAYCNLANALKEKGSVAEAEDCYNTALRLCPTHADSLNNLANIKREQGNIEEAVRLYRKALEVFPEFAAAHSNLASVLQQQGKLQEALMHYKEAIRISPTFADAYSNMGNTLKEMQDVQGALQCYTRAIQINPAFADAHSNLASIHKDSGNIPEAIASYRTALKLKPDFPDAYCNLAHCLQIVCDWTDYDERMKKLVSIVADQLEKNRLPSVHPHHSMLYPLSHGFRKAIAERHGNLCLDKINVLHKPPYEHPKDLKLSDGRLRVGYVSSDFGNHPTSHLMQSIPGMHNPDKFEVFCYALSPDDGTNFRVKVMAEANHFIDLSQIPCNGKAADRIHQDGIHILVNMNGYTKGARNELFALRPAPIQAMWLGYPGTSGALFMDYIITDQETSPAEVAEQYSEKLAYMPHTFFIGDHANMFPHLKKKAVIDFKSNGHIYDNRIVLNGIDLKAFLDSLPDVKIVKMKCPDGGDNADSSNTALNMPVIPMNTIAEAVIEMINRGQIQITINGFSISNGLATTQINNKAATGEEVPRTIIVTTRSQYGLPEDAIVYCNFNQLYKIDPSTLQMWANILKRVPNSVLWLLRFPAVGEPNIQQYAQNMGLPQNRIIFSPVAPKEEHVRRGQLADVCLDTPLCNGHTTGMDVLWAGTPMVTMPGETLASRVAASQLTCLGCLELIAKNRQEYEDIAVKLGTDLEYLKKVRGKVWKQRISSPLFNTKQYTMELERLYLQMWEHYAAGNKPDHMIKPVEVTESA

SEQ ID NO: 3 - DNA, 3141 bp, Artificial Sequence
  OGT antiparallel sequence/ OGT antisense DNA sequence
ttatgctgactcagtgacttcaacaggcttaatcatgtggtcaggtttgttgccagctgcataatgctcccacatctgtagatagagccgctctagttccattgtgtattgtttggtgttgaacagagggctagatattctttgcttccagactttgccacgaactttcttcaggtattctagatcagttcccagcttcacagctatgtcttcatattcttgtctgtttttagcaataagctcaagacaacctaagcaagtgagctgggatgctgcaactcgagaagcaagagtctctcctggcatagtcaccatgggggtccctgcccagaggacatccatccctgtggtgtgcccattacagagtggagtgtccaagcagacatcagccagctggcctctcctgacgtgttcctctttaggagcaacaggtgaaaaaatgatacggttctggggcaggcccatgttttgtgcatactgttgaatattaggttctcctactgctggaaaacgcaacagccagagtacactattgggaacacgcttcagaatgtttgcccacatctgcaaagtagaagggtcaattttatacaactgattaaagttacagtatacgatggcatcttctggtaacccgtactgagaacgggtggttacaataatggtacggggaacctcctctccagttgcagccttattgttgatctgagtagttgccagtccattgctaatactgaatccattaattgttatttgaatctgtcctcggttaatcatttcaataactgcttctgcaatagtattcataggaataacaggcatattaagagctgtgttactgctatctgcattgtctcctccatcaggacacttcatcttgacaattttcacatctggtagactatcaagaaatgctttgaggtcgatgccattcagaactatccgattgtcataaatgtgcccattggacttaaaatcgatgactgcttttttcttcaggtgagggaacatattagcatgatcaccaataaaaaaagtgtggggcatataagccaatttctcggaatactgctcagcaacttcagctggcgaagtttcctgatcagtgataatataatccatgaaaagcgcaccactcgtcccagggtatcccagccacattgcctgaataggagctggcctgagagcaaaaagctcatttcgagcgcccttagtatagccattcatatttacaaggatatgaattccatcctgatggatgcgatcagctgcttttccattgcatggaatctgagaaagatcaatgaaatgattggcttctgccatcaccttcactcggaagtttgtgccatcgtctgggctcagggcataacagaacacctcaaatttatcaggattgtgcatgcctggaatagactgcataaggtgagaagtaggatgattcccaaagtcggaactcacatatcctacacgcagccgaccatcactgagcttcaagtcttttggatgttcatatggtggtttatgaagaacattaatcttatctaagcacaggttgccgtgcctctcagcaatagccttcctgaagccatgagaaagaggatatagcatactatgatgaggatgcacagaaggcaacctattcttctctaactggtcagccacaatactgaccaacttcttcattcgctcatcatagtctgtccaatcacagacaatctgcaggcaatgagccaagttacaataagcatcaggaaaatcaggcttaagtttcagagccgtgcggtaagaagctatggcttctggaatattccctgaatccttatgaatggaagccagattgctatgtgcatctgcaaatgcaggattaatttggatggcacgcgtataacactgcaaggctccctgaacatcctgcatctcctttagagtgtttcccatattagagtaggcatcagcaaaggtaggactgattcgaatagcctccttataatgcatcagagcttcctgcagttttccctgctgctgcagtacacttgctaaatttgaatgggcagcagcaaactctgggaagacttctaatgctttacgatacaagcgaactgcctcttcaatgtttccctgttctcgtttgatattggctaggttattcagagagtctgcatgggtgggacacagacggagagctgtattataacaatcttctgcttcagcaacactgcccttctctttgagagcattggctaggttgcagtaagcatcagggaaatgtggttgtagttcgatagcccgcctgtaggtgtctattgccagatctatcaggccttgctcatagtatacacaagccaggttgccgtgcaccactgcgtgatttggactcaaacttagggcacgaagataagctgccacagctctgtcaaaaatgcgtgcctctttcaagacatttcctaaattgatataagcatccagaaagtttgggtcaagggtgacagccttttcaaagtgatgaattgcaagccaaatttccccttgtgcattgaaaacacagccaagattactccaagctactgcaaagttcggttgcgtctcaattgctttcaaataacatgccttggcttcttccaagcgacccagggctttgagcaggttccccaggtcactgcgaacacagtacaaatcaggattgtactgaagagcagagacgtaagcttgtactgccccttccatgtcacccgctgctaccaaggcggctgccaggttaatataaccatcgatgaaatcaggtttgagacgcaatgcatgtcgataatgctcaattgcctcctgcaactgccctctttccttgtacacattccccaaattcgaataagcttctgccagaagggggttctgtttaattgccagagtgctaaagtgagcagatctgtccagccttcgacactggaagtgtatagatgaaagtaataaaagcacaccagtattgtctggctcttgtctccagagctgcatgcagtgtctctcagctgcctcaaaatctcctgcctgatattctcgatgtgccaactcagctaacccttggaaggaaagcatacgtttcgttggttctgtgctgtcggccacgttgcccacggaagacgccat

SEQ ID NO: 4 - DNA, 19 bp, Artificial
  OGT antisense oligodeoxynucleotide Coding region “Code1PhT”
gtatcccagccacattgcc

SEQ ID NO: 5 - DNA, 21 bp, Artificial Sequence
  OGT antisense oligodeoxynucleotides Coding region “Code2PhT”
gcctcttcaatgtttccctgt

SEQ ID NO: 6 - DNA, 21 bp, Artificial Sequence
  OGT antisense oligodeoxynucleotides Coding region “Code3PhT”
cttctgccatcaccttcactc

SEQ ID NO: 7 - DNA, 19 bp, Artificial Sequence
  OGT antisense oligodeoxynucleotides 3′UTR region “3UTR1PhT”
ccctccacataaccatccc

SEQ ID NO: 8 - DNA, 19 bp, Artificial Sequence
  OGT antisense oligodeoxynucleotide 3′UTR region “3UTR2PhT”
gtgtctctccctaccatgc

SEQ ID NO: 9 - DNA, 21 bp, Artificial Sequence
  OGT antisense oligodeoxynucleotide Intron “INTR1PhT”
ctcctctaccatcccaaactc

SEQ ID NO: 10 - DNA, 20 bp, Artificial Sequence
  OGT antisense oligodeoxynucleotide Intron “INTR2PhT”
atcccagctactcaggaggc

SEQ ID NO: 11 - DNA, 20 bp, Artificial Sequence
  OGT antisense oligodeoxynucleotide Intron “INTR3PhT”
ttcctcctagagcctcccac

SEQ ID NO: 12 - DNA, 20 bp, Artificial Sequence
  OGT antisense oligodeoxynucleotides (coding region)
ttatgctgactcagtgactt

SEQ ID NO: 13 - DNA, 20 bp, Artificial Sequence
  OGT antisense oligodeoxynucleotide (coding region)
caacaggcttaatcatgtgg

SEQ ID NO: 14 - DNA, 20 bp, Artificial Sequence
  OGT antisense oligodeoxynucleotide (coding region)
tcaggtttgttgccagctgc

SEQ ID NO: 15 - DNA, 20 bp, Artificial Sequence
  OGT antisense oligodeoxynucleotides (coding region)
ataatgctcccacatctgta

SEQ ID NO: 16 - DNA, 20 bp, Artificial Sequence
  OGT antisense oligodeoxynucleotides (coding region)
gatagagccgctctagttcc

SEQ ID NO: 17 - DNA, 20 bp, Artificial Sequence
  OGT antisense oligodeoxynucleotides (coding region)
attgtgtattgtttggtgtt

SEQ ID NO: 18 - DNA, 20 bp, Artificial Sequence
  OGT antisense oligodeoxynucleotide (coding region)
gaacagagggctagatattc

SEQ ID NO: 19 - DNA, 20 bp, Artificial Sequence
  OGT antisense oligodeoxynucleotide (coding region)
tttgcttccagactttgcca

SEQ ID NO: 20 - DNA, 20 bp, Artificial Sequence
  OGT antisense oligodeoxynucleotide (coding region)
cgaactttcttcaggtattc

SEQ ID NO: 21 - DNA, 20 bp, Artificial Sequence
  OGT antisense oligodeoxynucleotide (coding region)
tagatcagttcccagcttca

SEQ ID NO: 22 - DNA, 20 bp, Artificial Sequence
  OGT antisense oligodeoxynucleotide (coding region)
cagctatgtcttcatattct

SEQ ID NO: 23 - DNA, 20 bp, Artificial Sequence
  OGT antisense oligodeoxynucleotide (coding region)
tgtctgtttttagcaataag

SEQ ID NO: 24 - DNA, 20 bp, Artificial Sequence
  OGT antisense oligodeoxynucleotide (coding region)
ctcaagacaacctaagcaag

SEQ ID NO: 25 - DNA, 20 bp, Artificial Sequence
  OGT antisense oligodeoxynucleotide (coding region)
tgagctgggatgctgcaact

SEQ ID NO: 26 - DNA, 20 bp, Artificial Sequence
  OGT antisense oligodeoxynucleotide (coding region)
cgagaagcaagagtctctcc

SEQ ID NO: 27 - DNA, 20 bp, Artificial Sequence
  OGT antisense oligodeoxynucleotide (coding region)
tggcatagtcaccatggggg

SEQ ID NO: 28 - DNA, 20 bp, Artificial Sequence
  OGT antisense oligodeoxynucleotide (coding region)
tccctgcccagaggacatcc

SEQ ID NO: 29 - DNA, 20 bp, Artificial Sequence
  OGT antisense oligodeoxynucleotide (coding region)
atccctgtggtgtgcccatt

SEQ ID NO: 30 - DNA, 20 bp, Artificial Sequence
  OGT antisense oligodeoxynucleotide (coding region)
acagagtggagtgtccaagc

SEQ ID NO: 31 - DNA, 20 bp, Artificial Sequence
  OGT antisense oligodeoxynucleotide (coding region)
agacatcagccagctggcct

SEQ ID NO: 32 - DNA, 20 bp, Artificial Sequence
  OGT antisense oligodeoxynucleotide (coding region)
ctcctgacgtgttcctcttt

SEQ ID NO: 33 - DNA, 20 bp, Artificial Sequence
  OGT antisense oligodeoxynucleotide (coding region)
aggagcaacaggtgaaaaaa

SEQ ID NO: 34 - DNA, 20 bp, Artificial Sequence
  OGT antisense oligodeoxynucleotide (coding region)
tgatacggttctggggcagg

SEQ ID NO: 35 - DNA, 20 bp, Artificial Sequence
  OGT antisense oligodeoxynucleotide (coding region)
cccatgttttgtgcatactg

SEQ ID NO: 36 - DNA, 20 bp, Artificial Sequence
  OGT antisense oligodeoxynucleotide (coding region)
ttgaatattaggttctccta

SEQ ID NO: 37 - DNA, 20 bp, Artificial Sequence
  OGT antisense oligodeoxynucleotide (coding region)
ctgctggaaaacgcaacagc

SEQ ID NO: 38 - DNA, 20 bp, Artificial Sequence
  OGT antisense oligodeoxynucleotide (coding region)
cagagtacactattgggaac

SEQ ID NO: 39 - DNA, 20 bp, Artificial Sequence
  OGT antisense oligodeoxynucleotide (coding region)
acgcttcagaatgtttgccc

SEQ ID NO: 40 - DNA, 20 bp, Artificial Sequence
  OGT antisense oligodeoxynucleotide (coding region)
acatctgcaaagtagaaggg

SEQ ID NO: 41 - DNA, 20 bp, Artificial Sequence
  OGT antisense oligodeoxynucleotide (coding region)
tcaattttatacaactgatt

SEQ ID NO: 42 - DNA, 20 bp, Artificial Sequence
  OGT antisense oligodeoxynucleotide (coding region)
aaagttacagtatacgatgg

SEQ ID NO: 43 - DNA, 20 bp, Artificial Sequence
  OGT antisense oligodeoxynucleotide (coding region)
catcttctggtaacccgtac

SEQ ID NO: 44 - DNA, 20 bp, Artificial Sequence
  OGT antisense oligodeoxynucleotide (coding region)
tgagaacgggtggttacaat

SEQ ID NO: 45 - DNA, 20 bp, Artificial Sequence
  OGT antisense oligodeoxynucleotide (coding region)
aatggtacggggaacctcct

SEQ ID NO: 46 - DNA, 20 bp, Artificial Sequence
  OGT antisense oligodeoxynucleotide (coding region)
ctccagttgcagccttattg

SEQ ID NO: 47 - DNA, 20 bp, Artificial Sequence
  OGT antisense oligodeoxynucleotide (coding region)
ttgatctgagtagttgccag

SEQ ID NO: 48 - DNA, 20 bp, Artificial Sequence
  OGT antisense oligodeoxynucleotide (coding region)
tccattgctaatactgaatc

SEQ ID NO: 49 - DNA, 20 bp, Artificial Sequence
  OGT antisense oligodeoxynucleotide (coding region)
cattaattgttatttgaatc

SEQ ID NO: 50 - DNA, 20 bp, Artificial Sequence
  OGT antisense oligodeoxynucleotide (coding region)
tgtcctcggttaatcatttc

SEQ ID NO: 51 - DNA, 20 bp, Artificial Sequence
  OGT antisense oligodeoxynucleotide (coding region)
aataactgcttctgcaatag

SEQ ID NO: 52 - DNA, 20 bp, Artificial Sequence
  OGT antisense oligodeoxynucleotide (coding region)
tattcataggaataacaggc

SEQ ID NO: 53 - DNA, 20 bp, Artificial Sequence
  OGT antisense oligodeoxynucleotide (coding region)
atattaagagctgtgttact

SEQ ID NO: 54 - DNA, 20 bp, Artificial Sequence
  OGT antisense oligodeoxynucleotide (coding region)
gctatctgcattgtctcctc

SEQ ID NO: 55 - DNA, 20 bp, Artificial Sequence
  OGT antisense oligodeoxynucleotide (coding region)
catcaggacacttcatcttg

SEQ ID NO: 56 - DNA, 20 bp, Artificial Sequence
  OGT antisense oligodeoxynucleotide (coding region)
acaattttcacatctggtag

SEQ ID NO: 57 - DNA, 20 bp, Artificial Sequence
  OGT antisense oligodeoxynucleotide (coding region)
actatcaagaaatgctttga

SEQ ID NO: 58 - DNA, 20 bp, Artificial Sequence
  OGT antisense oligodeoxynucleotide (coding region)
ggtcgatgccattcagaact

SEQ ID NO: 59 - DNA, 20 bp, Artificial Sequence
  OGT antisense oligodeoxynucleotide (coding region)
atccgattgtcataaatgtg

SEQ ID NO: 60 - DNA, 20 bp, Artificial Sequence
  OGT antisense oligodeoxynucleotide (coding region)
cccattggacttaaaatcga

SEQ ID NO: 61 - DNA, 20 bp, Artificial Sequence
  OGT antisense oligodeoxynucleotide (coding region)
tgactgcttttttcttcagg

SEQ ID NO: 62 - DNA, 20 bp, Artificial Sequence
  OGT antisense oligodeoxynucleotide (coding region)
tgagggaacatattagcatg

SEQ ID NO: 63 - DNA, 20 bp, Artificial Sequence
  OGT antisense oligodeoxynucleotide (coding region)
atcaccaataaaaaaagtgt

SEQ ID NO: 64 - DNA, 20 bp, Artificial Sequence
  OGT antisense oligodeoxynucleotide (coding region)
ggggcatataagccaatttc

SEQ ID NO: 65 - DNA, 20 bp, Artificial Sequence
  OGT antisense oligodeoxynucleotide (coding region)
tcggaatactgctcagcaac

SEQ ID NO: 66 - DNA, 20 bp, Artificial Sequence
  OGT antisense oligodeoxynucleotide (coding region)
ttcagctggcgaagtttcct

SEQ ID NO: 67 - DNA, 20 bp, Artificial Sequence
  OGT antisense oligodeoxynucleotide (coding region)
gatcagtgataatataatcc

SEQ ID NO: 68 - DNA, 20 bp, Artificial Sequence
  OGT antisense oligodeoxynucleotide (coding region)
atgaaaagcgcaccactcgt

SEQ ID NO: 69 - DNA, 20 bp, Artificial Sequence
  OGT antisense oligodeoxynucleotide (coding region)
cccagggtatcccagccaca

SEQ ID NO: 70 - DNA, 20 bp, Artificial Sequence
  OGT antisense oligodeoxynucleotide (coding region)
ttgcctgaataggagctggc

SEQ ID NO: 71 - DNA, 20 bp, Artificial Sequence
  OGT antisense oligodeoxynucleotide (coding region)
ctgagagcaaaaagctcatt

SEQ ID NO: 72 - DNA, 20 bp, Artificial Sequence
  OGT antisense oligodeoxynucleotide (coding region)
tcgagcgcccttagtatagc

SEQ ID NO: 73 - DNA, 20 bp, Artificial Sequence
  OGT antisense oligodeoxynucleotide (coding region)
cattcatatttacaaggata

SEQ ID NO: 74 - DNA, 20 bp, Artificial Sequence
  OGT antisense oligodeoxynucleotide (coding region)
tgaattccatcctgatggat

SEQ ID NO: 75 - DNA, 20 bp, Artificial Sequence
  OGT antisense oligodeoxynucleotide (coding region)
gcgatcagctgcttttccat

SEQ ID NO: 76 - DNA, 20 bp, Artificial Sequence
  OGT antisense oligodeoxynucleotide (coding region)
tgcatggaatctgagaaaga

SEQ ID NO: 77 - DNA, 20 bp, Artificial Sequence
  OGT antisense oligodeoxynucleotide (coding region)
tcaatgaaatgattggcttc

SEQ ID NO: 78 - DNA, 20 bp, Artificial Sequence
  OGT antisense oligodeoxynucleotide (coding region)
tgccatcaccttcactcgga

SEQ ID NO: 79 - DNA, 20 bp, Artificial Sequence
  OGT antisense oligodeoxynucleotide (coding region)
agtttgtgccatcgtctggg

SEQ ID NO: 80 - DNA, 20 bp, Artificial Sequence
  OGT antisense oligodeoxynucleotide (coding region)
ctcagggcataacagaacac

SEQ ID NO: 81 - DNA, 20 bp, Artificial Sequence
  OGT antisense oligodeoxynucleotide (coding region)
ctcaaatttatcaggattgt

SEQ ID NO: 82 - DNA, 20 bp, Artificial Sequence
  OGT antisense oligodeoxynucleotide (coding region)
gcatgcctggaatagactgc

SEQ ID NO: 83 - DNA, 20 bp, Artificial Sequence
  OGT antisense oligodeoxynucleotide (coding region)
ataaggtgagaagtaggatg

SEQ ID NO: 84 - DNA, 20 bp, Artificial Sequence
  OGT antisense oligodeoxynucleotide (coding region)
attcccaaagtcggaactca

SEQ ID NO: 85 - DNA, 20 bp, Artificial Sequence
  OGT antisense oligodeoxynucleotide (coding region)
catatcctacacgcagccga

SEQ ID NO: 86 - DNA, 20 bp, Artificial Sequence
  OGT antisense oligodeoxynucleotide (coding region)
ccatcactgagcttcaagtc

SEQ ID NO: 87 - DNA, 20 bp, Artificial Sequence
  OGT antisense oligodeoxynucleotide (coding region)
ttttggatgttcatatggtg

SEQ ID NO: 88 - DNA, 20 bp, Artificial Sequence
  OGT antisense oligodeoxynucleotide (coding region)
gtttatgaagaacattaatc

SEQ ID NO: 89 - DNA, 20 bp, Artificial Sequence
  OGT antisense oligodeoxynucleotide (coding region)
ttatctaagcacaggttgcc

SEQ ID NO: 90 - DNA, 20 bp, Artificial Sequence
  OGT antisense oligodeoxynucleotide (coding region)
gtgcctctcagcaatagcct

SEQ ID NO: 91 - DNA, 20 bp, Artificial Sequence
  OGT antisense oligodeoxynucleotide (coding region)
tcctgaagccatgagaaaga

SEQ ID NO: 92 - DNA, 20 bp, Artificial Sequence
  OGT antisense oligodeoxynucleotide (coding region)
ggatatagcatactatgatg

SEQ ID NO: 93 - DNA, 20 bp, Artificial Sequence
  OGT antisense oligodeoxynucleotide (coding region)
aggatgcacagaaggcaacc

SEQ ID NO: 94 - DNA, 20 bp, Artificial Sequence
  OGT antisense oligodeoxynucleotide (coding region)
tattcttctctaactggtca

SEQ ID NO: 95 - DNA, 20 bp, Artificial Sequence
  OGT antisense oligodeoxynucleotide (coding region)
gccacaatactgaccaactt

SEQ ID NO: 96 - DNA, 20 bp, Artificial Sequence
  OGT antisense oligodeoxynucleotide (coding region)
cttcattcgctcatcatagt

SEQ ID NO: 97 - DNA, 20 bp, Artificial Sequence
  OGT antisense oligodeoxynucleotide (coding region)
ctgtccaatcacagacaatc

SEQ ID NO: 98 - DNA, 20 bp, Artificial Sequence
  OGT antisense oligodeoxynucleotide (coding region)
tgcaggcaatgagccaagtt

SEQ ID NO: 99 - DNA, 20 bp, Artificial Sequence
  OGT antisense oligodeoxynucleotide (coding region)
acaataagcatcaggaaaat

SEQ ID NO: 100 - DNA, 20 bp, Artificial Sequence
  OGT antisense oligodeoxynucleotide (coding region)
caggcttaagtttcagagcc

SEQ ID NO: 101 - DNA, 20 bp, Artificial Sequence
  OGT antisense oligodeoxynucleotide (coding region)
gtgcggtaagaagctatggc

SEQ ID NO: 102 - DNA, 20 bp, Artificial Sequence
  OGT antisense oligodeoxynucleotide (coding region)
ttctggaatattccctgaat

SEQ ID NO: 103 - DNA, 20 bp, Artificial Sequence
  OGT antisense oligodeoxynucleotide (coding region)
ccttatgaatggaagccaga

SEQ ID NO: 104 - DNA, 20 bp, Artificial Sequence
  OGT antisense oligodeoxynucleotide (coding region)
ttgctatgtgcatctgcaaa

SEQ ID NO: 105 - DNA, 20 bp, Artificial Sequence
  OGT antisense oligodeoxynucleotide (coding region)
tgcaggattaatttggatgg

SEQ ID NO: 106 - DNA, 20 bp, Artificial Sequence
  OGT antisense oligodeoxynucleotide (coding region)
cacgcgtataacactgcaag

SEQ ID NO: 107 - DNA, 20 bp, Artificial Sequence
  OGT antisense oligodeoxynucleotide (coding region)
gctccctgaacatcctgcat

SEQ ID NO: 108 - DNA, 20 bp, Artificial Sequence
  OGT antisense oligodeoxynucleotide (coding region)
ctcctttagagtgtttccca

SEQ ID NO: 109 - DNA, 20 bp, Artificial Sequence
  OGT antisense oligodeoxynucleotide (coding region)
tattagagtaggcatcagca

SEQ ID NO: 110 - DNA, 20 bp, Artificial Sequence
  OGT antisense oligodeoxynucleotide (coding region)
aaggtaggactgattcgaat

SEQ ID NO: 111 - DNA, 20 bp, Artificial Sequence
  OGT antisense oligodeoxynucleotide (coding region)
agcctccttataatgcatca

SEQ ID NO: 112 - DNA, 20 bp, Artificial Sequence
  OGT antisense oligodeoxynucleotide (coding region)
gagcttcctgcagttttccc

SEQ ID NO: 113 - DNA, 20 bp, Artificial Sequence
  OGT antisense oligodeoxynucleotide (coding region)
tgctgctgcagtacacttgc

SEQ ID NO: 114 - DNA, 20 bp, Artificial Sequence
  OGT antisense oligodeoxynucleotide (coding region)
taaatttgaatgggcagcag

SEQ ID NO: 115 - DNA, 20 bp, Artificial Sequence
  OGT antisense oligodeoxynucleotide (coding region)
caaactctgggaagacttct

SEQ ID NO: 116 - DNA, 20 bp, Artificial Sequence
  OGT antisense oligodeoxynucleotide (coding region)
aatgctttacgatacaagcg

SEQ ID NO: 117 - DNA, 20 bp, Artificial Sequence
  OGT antisense oligodeoxynucleotide (coding region)
aactgcctcttcaatgtttc

SEQ ID NO: 118 - DNA, 20 bp, Artificial Sequence
  OGT antisense oligodeoxynucleotide (coding region)
cctgttctcgtttgatattg

SEQ ID NO: 119 - DNA, 20 bp, Artificial Sequence
  OGT antisense oligodeoxynucleotide (coding region)
gctaggttattcagagagtc

SEQ ID NO: 120 - DNA, 20 bp, Artificial Sequence
  OGT antisense oligodeoxynucleotide (coding region)
tgcatgggtgggacacagac

SEQ ID NO: 121 - DNA, 20 bp, Artificial Sequence
  OGT antisense oligodeoxynucleotide (coding region)
ggagagctgtattataacaa

SEQ ID NO: 122 - DNA, 20 bp, Artificial Sequence
  OGT antisense oligodeoxynucleotide (coding region)
tcttctgcttcagcaacact

SEQ ID NO: 123 - DNA, 20 bp, Artificial Sequence
  OGT antisense oligodeoxynucleotide (coding region)
gcccttctctttgagagcat

SEQ ID NO: 124 - DNA, 20 bp, Artificial Sequence
  OGT antisense oligodeoxynucleotide (coding region)
tggctaggttgcagtaagca

SEQ ID NO: 125 - DNA, 20 bp, Artificial Sequence
  OGT antisense oligodeoxynucleotide (coding region)
tcagggaaatgtggttgtag

SEQ ID NO: 126 - DNA, 20 bp, Artificial Sequence
  OGT antisense oligodeoxynucleotide (coding region)
ttcgatagcccgcctgtagg

SEQ ID NO: 127 - DNA, 20 bp, Artificial Sequence
  OGT antisense oligodeoxynucleotide (coding region)
tgtctattgccagatctatc

SEQ ID NO: 128 - DNA, 20 bp, Artificial Sequence
  OGT antisense oligodeoxynucleotide (coding region)
aggccttgctcatagtatac

SEQ ID NO: 129 - DNA, 20 bp, Artificial Sequence
  OGT antisense oligodeoxynucleotide (coding region)
acaagccaggttgccgtgca

SEQ ID NO: 130 - DNA, 20 bp, Artificial Sequence
  OGT antisense oligodeoxynucleotide (coding region)
ccactgcgtgatttggactc

SEQ ID NO: 131 - DNA, 20 bp, Artificial Sequence
  OGT antisense oligodeoxynucleotide (coding region)
aaacttagggcacgaagata

SEQ ID NO: 132 - DNA, 20 bp, Artificial Sequence
  OGT antisense oligodeoxynucleotide (coding region)
agctgccacagctctgtcaa

SEQ ID NO: 133 - DNA, 20 bp, Artificial Sequence
  OGT antisense oligodeoxynucleotide (coding region)
aaatgcgtgcctctttcaag

SEQ ID NO: 134 - DNA, 20 bp, Artificial Sequence
  OGT antisense oligodeoxynucleotide (coding region)
acatttcctaaattgatata

SEQ ID NO: 135 - DNA, 20 bp, Artificial Sequence
  OGT antisense oligodeoxynucleotide (coding region)
agcatccagaaagtttgggt

SEQ ID NO: 136 - DNA, 20 bp, Artificial Sequence
  OGT antisense oligodeoxynucleotide (coding region)
caagggtgacagccttttca

SEQ ID NO: 137 - DNA, 20 bp, Artificial Sequence
  OGT antisense oligodeoxynucleotide (coding region)
aagtgatgaattgcaagcca

SEQ ID NO: 138 - DNA, 20 bp, Artificial Sequence
  OGT antisense oligodeoxynucleotide (coding region)
aatttccccttgtgcattga

SEQ ID NO: 139 - DNA, 20 bp, Artificial Sequence
  OGT antisense oligodeoxynucleotide (coding region)
aaacacagccaagattactc

SEQ ID NO: 140 - DNA, 20 bp, Artificial Sequence
  OGT antisense oligodeoxynucleotide (coding region)
caagctactgcaaagttcgg

SEQ ID NO: 141 - DNA, 20 bp, Artificial Sequence
  OGT antisense oligodeoxynucleotide (coding region)
ttgcgtctcaattgctttca

SEQ ID NO: 142 - DNA, 20 bp, Artificial Sequence
  OGT antisense oligodeoxynucleotide (coding region)
aataacatgccttggcttct

SEQ ID NO: 143 - DNA, 20 bp, Artificial Sequence
  OGT antisense oligodeoxynucleotide (coding region)
tccaagcgacccagggcttt

SEQ ID NO: 144 - DNA, 20 bp, Artificial Sequence
  OGT antisense oligodeoxynucleotide (coding region)
gagcaggttccccaggtcac

SEQ ID NO: 145 - DNA, 20 bp, Artificial Sequence
  OGT antisense oligodeoxynucleotide (coding region)
tgcgaacacagtacaaatca

SEQ ID NO: 146 - DNA, 20 bp, Artificial Sequence
  OGT antisense oligodeoxynucleotide (coding region)
ggattgtactgaagagcaga

SEQ ID NO: 147 - DNA, 20 bp, Artificial Sequence
  OGT antisense oligodeoxynucleotide (coding region)
gacgtaagcttgtactgccc

SEQ ID NO: 148 - DNA, 20 bp, Artificial Sequence
  OGT antisense oligodeoxynucleotide (coding region)
cttccatgtcacccgctgct

SEQ ID NO: 149 - DNA, 20 bp, Artificial Sequence
  OGT antisense oligodeoxynucleotide (coding region)
accaaggcggctgccaggtt

SEQ ID NO: 150 - DNA, 20 bp, Artificial Sequence
  OGT antisense oligodeoxynucleotide (coding region)
aatataaccatcgatgaaat

SEQ ID NO: 151 - DNA, 20 bp, Artificial Sequence
  OGT antisense oligodeoxynucleotide (coding region)
caggtttgagacgcaatgca

SEQ ID NO: 152 - DNA, 20 bp, Artificial Sequence
  OGT antisense oligodeoxynucleotide (coding region)
tgtcgataatgctcaattgc

SEQ ID NO: 153 - DNA, 20 bp, Artificial Sequence
  OGT antisense oligodeoxynucleotide (coding region)
ctcctgcaactgccctcttt

SEQ ID NO: 154 - DNA, 20 bp, Artificial Sequence
  OGT antisense oligodeoxynucleotide (coding region)
ccttgtacacattccccaaa

SEQ ID NO: 155 - DNA, 20 bp, Artificial Sequence
  OGT antisense oligodeoxynucleotide (coding region)
ttcgaataagcttctgccag

SEQ ID NO: 156 - DNA, 20 bp, Artificial Sequence
  OGT antisense oligodeoxynucleotide (coding region)
aagggggttctgtttaattg

SEQ ID NO: 157 - DNA, 20 bp, Artificial Sequence
  OGT antisense oligodeoxynucleotide (coding region)
ccagagtgctaaagtgagca

SEQ ID NO: 158 - DNA, 20 bp, Artificial Sequence
  OGT antisense oligodeoxynucleotide (coding region)
gatctgtccagccttcgaca

SEQ ID NO: 159 - DNA, 20 bp, Artificial Sequence
  OGT antisense oligodeoxynucleotide (coding region)
ctggaagtgtatagatgaaa

SEQ ID NO: 160 - DNA, 20 bp, Artificial Sequence
  OGT antisense oligodeoxynucleotide (coding region)
gtaataaaagcacaccagta

SEQ ID NO: 161 - DNA, 20 bp, Artificial Sequence
  OGT antisense oligodeoxynucleotide (coding region)
ttgtctggctcttgtctcca

SEQ ID NO: 162 - DNA, 20 bp, Artificial Sequence
  OGT antisense oligodeoxynucleotide (coding region)
gagctgcatgcagtgtctct

SEQ ID NO: 163 - DNA, 20 bp, Artificial Sequence
  OGT antisense oligodeoxynucleotide (coding region)
cagctgcctcaaaatctcct

SEQ ID NO: 164 - DNA, 20 bp, Artificial Sequence
  OGT antisense oligodeoxynucleotide (coding region)
gcctgatattctcgatgtgc

SEQ ID NO: 165 - DNA, 20 bp, Artificial Sequence
  OGT antisense oligodeoxynucleotide (coding region)
caactcagctaacccttgga

SEQ ID NO: 166 - DNA, 20 bp, Artificial Sequence
  OGT antisense oligodeoxynucleotide (coding region)
aggaaagcatacgtttcgtt

SEQ ID NO: 167 - DNA, 20 bp, Artificial Sequence
  OGT antisense oligodeoxynucleotide (coding region)
ggttctgtgctgtcggccac

SEQ ID NO: 168 - DNA, 21 bp, Artificial Sequence
  OGT antisense oligodeoxynucleotide (coding region)
gttgcccacggaagacgccat

SEQ ID NO: 169 - RNA, 19 nt, Artificial Sequence
  OGT siRNA1 19mer, pos. 740
gcauguuauuugaaagcaa

SEQ ID NO: 170 - RNA, 19 nt, Artificial Sequence
  OGT siRNA2 19mer, pos. 1948
gcacaauccugauaaauuu

SEQ ID NO: 171 - RNA, 19 nt, Artificial Sequence
  OGT siRNA3 19mer, pos. 959
gcccuaaguuugaguccaa

SEQ ID NO: 172 - DNA, 21 bp, Artificial Sequence
  OGT shRNA
gccctaagtttgagtccaaat

SEQ ID NO: 173 - DNA, 49836 bp, Artificial Sequence
  OGT Sense DNA sequence/Nucleotide Sequence - full sequence including regulatory regions, introns, and coding sequence
attgtcctttcacatctggttgctcataaaagcaaaacctaccattcgagtgttcaattctagtgtgaagtgttttaccatgggagcaaaagcttgagcttgaaagataacagtcattatggctatctgggacaagaaaaaaaccatctagcacatttgctcacttttcttctgcctgtcagtgtatgattgcccccagcaccattattattttgcaggttttttcagctcctctgtaaggcttgtcacaaccatagtgtcactactttggacagaatcatcaactcttgcaaccccaggaggacagtcaggattagaattcaaatgacagtgggtactttcagacacacacatagacaactgcacctgtgagtccacagaagttcctttagattctgtattggaggtagtaatgatgaaagtgtcttcaggaatgtcttcatggcttgctcttgggctctgcattgtaactccttacaggtaccaatgaacaagccattcagagatggatccacaaagatctctggagcaacgaccaggtcctgctctacccgattctcagctttggggatagaattcccagaaacatctgtgtccggagttggttccttccagtgggttcttggtcttgctgacttcaagaatgaagccgtggaccttcgcggtgagtgttacagctctttaaaggtagcacagacccaaagagtgagcggcagcaagatttattgtgaagagcaaaagaacaaagcttccatagcgtgaaagggtacccaagcaggttgccagtggggctgggggcggccagcatttattcctttatttgtccccgcccacatcctgctgattggtccattttacagagtgctgattggtccattttacagagtgctctttggtgtttttacaattctttagctagacacagagcgctaattggtgcattttacagagtgctgattggtgcatttacaatcctttagctagaaacagagcgctgattggtgcgttttgtttttgtttttttttttgagacagagtctcgctctgtcgcccaggctggagtgcagtggtgcaatctcagctcactgcaacctccgcctcctgggttcatgccattctcatgcctcagcctcccaagtagctgggactacaggtgcccgccaccacgcctgactaattttttgtatttttagtagagacggggtttcaccatgttagccaggatggtctccatctcctgacctcatgatctgcctgcttcagcctcccaaagtgctgggattacaggtgtgagccactgcacctggctggtgcatttttacagagtgctgattggtgcatttacaatcctttagctaaacacagagtgctgattggtgcgttttacaatcttcttgtaagacacaaaagttctccaagtccccacccaacccagaagtccagctggcttcaccactcacttccatgggagaactgtccagacaatagcatgacacccttgcaaaggataaagcctctcatctaaaggcatagaatactgaatatagtcttgaggcataagtccaataagtacaggcacatgttccttgaagtgaagatgcaagtctccattctgaaattttgaactctttactttgttctttgtatgcagcttcaggctgaagatagtgaaaatccgtgccagacttggattggaagaatgaaccaaggctttgagtctcttcgtctcatctccgttttgatacgtgtctccttagtttgtaagccaaggagaggactacagtgaggattgtggagggaagtggaccttattggtctttgagctctcacatctttaaagactgttggtgctgagagaaaaagtatcctcatctggcagaactcccatagggtgtgctccccttgctgttctgcttttgttttgcagaaagctgcctttttcaatgtactcattaggctttcattgttttttccttttttacctttttcatctttcactgttgatatcacaggtggccatatctttaccagtgcagctaccaaataagaaatcatcttttccacattcactagttagtgatggttgttgtactaccatgaaatcagtttcttctttacttttattcaggttacaagatttctggaaggttttaagatcaattttaattagtctttgtgactggttacttcatccaggggaccaatttctctctggaatattagtcccgcacccctggagagcctgtgcggctgcatctccctccggaggtggttctggagttggttccttttggtgggttcctggtctcgctgacttcaagaatgaagccacggaccttcacggtgagtgttacagctcttaaaggtggcacggactcaaagagtgagcagcagcaagattcattgtgaagacggaaagaacacagcttccacggtgtggaaagagacccgagtgggttgctactactggcctggtgggtgggtgggtgggtgggtgcggaatgtggccagcttttattcccttatttgtccccacccatgtcctgctgattggtccattttacagagtgctgattggtccattttacaaggtgctaattggtccattttacagagcgctgattggtccattttacagactgctgattggtccattttacaaacctctagctagccacagagcactgattggtgtgtttttacagagcactgattggcgcattttacaaacctctagctagccacagagcgctgattggtgcgttttacaatcctagctacagagtgctgattggtgcattttacaatcctcttgtaagacagaaaagttctttaagtccccacccaacccaggaagtccagctggcttctcctctcacatctatgtatttctttggcttcatttaacctttggagccattttttttcagcagcagtgcaacagcagccaccagcccgggtctcatccagcgctggtgctactgctgtgaaatgtgtgttgttctgagcagctgtatttgtgatactttgttacacagcagcaaaaaaagtaatgcaacatcagtggtgttactgggtgacaaagtttgggattggggttgaagacccagctgatttaattataattggttctaaatttaaaattgcacataaaaaaaacccagaaatcaatgtgaacaacaataaaaaacatacaaaagcacttccttcaatgtctagatcaaggcttctttctcaccccctccaaccttacctgtttcctccctagatattcatagagagtaatatattttatcatctaaatagtcttctgtattgctctctagtagctttttgtgtctacctaactaggcttcagattccttgagggcgaggatttctgttcatttttgtgccccccacagtgccttgcacatagagactctagtaaatgttacttattggctgaagaattaggaacatgacctgcatttaacaacaaaaagagtttttgttaattagctagcacccgctcatcagcaaggcctaaacatctgccagtggaatctgagaagtctcttctaaaagttgaaaagctgagtttactcattacagatagggaacttgtagtaggtgtgtgtgtgcgggggggaggttttaccagttaaatcaaattacaagaggttggtgctcaattttcaggagagtttgtgtagtgatttttgtctttttcactgcatgatgtgactcttgattatgggatggaagaaattagcattatgaattttctctgtgccatttccataattaattgtttgttgactgttttgaagaaatttaagcagaatttgaggacttagttttaaaaaatgtagtgaaggggctaggtagcaaaaactttttgaagaaattcattgacctgctttcttagatcaaggactttttgaaataaaaaggctctcagagatccaacttgtagcctccagtggcttgtaatactttcttgctgatgtggcaagctaccttaagtcgtttctagaacaagaggtgaagtataaataccaatgtgacaaaatgtctgaacagtagacttgaaactgcaggaagatcgtaagaaccgatttgttcaaggaagtagaggcaattcatgactcaatctagacgtgtggtttaggcagatggcaagtgaattcatttatcattgttgatctgctttgagctcagtggaatcgccgactgtgacaatgtgcagagctagctggtggaaatgaagtcacaactcgaggagagtcatggattttgacgaacgtgaatcacctttaacatttttatggctgccttcggagtgcaaacgcccagcatgtccctgagttattaaacatttgccctatcaccgtgggcaggagtattctgaataccaaaacgattttgaaatttcaggctatacggccaacattgaaggtgtctgcattcaactggccctgtgaaagcttcagtctttttcatcgaagtgctgaaatcgatggccgagtcacaatgaagacgggctcaaatcggaaccctctcgctcctcgttttgagcagctgtctttgagaggcaatcttctgaattgactggctcagaattgatccgtgagagtcgtaaccatccgtttttgtacgaatacagttgataaacttttttagacgcctttggcatttatgtttaaaacgacaccacctgctaagtaacaaacactcttcttaatcgcggtggcaaaaacgtatccgtcccgtgtgtgtttgtttttacccttccgctacactcttaagaaacttggccgcttccgccccagagagtgccctaaccacgagtcgggttgacagggctgctcgcatgcgcggtctccaccgtggtgtttgtcgcaatgggcgctgcgcggaggactacatggaatctcgactttgcagcttgaagtcattttgttggcggcgctggttaccggctacctccgcgcgggcacttgattgtaacagaaagtagttccggcccatgttgtttcggccgaggagccgtcgccgccatttcaagaccgtactaggtagatggtcaattagagttcccagggtttgaagcctgtaactgctgccgccgctcaagccctccagagcattgctacggctgctgcccttgtactactacctccaaatacgttcttgctggtagtggcggcagcaggaccaattacctcttttttgctctccctcgagaagctccagatggcgtcttccgtgggcaacgtggccgacagcacaggtaccggtgtcccgttctaccttggcagatgcccccttggggtctcgcgccgtcctctaccttgtatcactccttccctcccttccctcgaaccatccccattacatcagtgacattgctaagcttttcgtggtctactggcatctgatcacatcgggttttccgctgttgtccgcttcccgaacatttcctcctcctcccttgcttgctttttgctgcctttcttgctttgtttttatttcgtaaatgttctagcattccactctctccaggcgcttttatcacacgtgcgcgcttagaatgtcgtggaatggactgcattggaatccctgccctttcactcctttgcagcggttttctcccctaattccagctcatcgcctctcctttttcccctccatctcctccttcatctccccccccagtcttttccccttagccagatttggtccttctctgccaatctagggcgggggtgccgttccgatgtaatcatatcctcactttgcatcacccgaggacgatcaaaagctcgtcgatggtgccacaagtcacctcgagaagaggggacattgagcagcaccctgctaggcagtgcctttctggtgttctttcggttgagcggtcccggtttagattggaaatttccaggggtttcctttgccatcgcccaattccaccgctgccctcagcctggatttcccaccctgtccgcggggaaccacacccttcccttcgcgaggttgccagccctgttcacttcctatccttgggggagaaaccaccccgatgctgctgctttggcgttgaccaccctgcccgcccaattctcgctttgtgttactttctcgatgtattcctccgccgccgcctctgggtctgccatccccattcttcagcccagtatctctttattcacagggtgtaatattcttttaagtttccatccctgcttgaagagtttgtaaagcaactggagtgtagctgcacaagtacaaggcggttgcccttttagacacttcagtcgcgagcatttggtactgaaaatacgaaaacaaccaaattgtaggagtgtccttcagctgcctttccatattgaggcgatttgggcggaagggggtggggaattgggctgcgcttctgaggtttcccgcgaggttattttttggttgtttctcagttgagatgtgtggagggttttagctactgttcctggaagttgtgctgtaagtagtaggtgaatcgtcaatagtgtcttatccgtaattgtagggagtgggattaataattagtccgtgctcctctccccacgtctccaccgtgattttcaaaggtttaagattctgtactggattctgtactagcttgccctttttaatgtgttatttatttgagaaagctgtcgtttcaagtgttaaaatagcagataagcggcattgaaaagtgacaaagtaatgtgtgaaaacaaggagctaggaagttctagagacttagctaaaatgtaattttagtttctattaaagatataacctctcccagatgtggatgcaagtctcaatgctctttgtgttggtggtcagctgtgcattctgtcaggaaatttactttgctttcctcagtatccggaacgctcatcagctctgatgattggggagctgggctcaaatttgaactagtcaccttaaccttggtagaattaaataatttgtccatggtttcccatttcaaccagctagtgaagtgctacggtcgaattcccagtttaagtatttacagttgtgacaagcagttttttttttttttttttccctctctgggctttagctatatcctttaaagatctttatgtatctggattaagtcacttgcagcgaagtgcttttatttattcattatgttgcgctttatcaaatatattttagcgtccatcctccctccctcccaacgttttgaccattgcagttgcaggtaactgcctggcttatataggcttgtttgcaatgtcttaatcctttagtcaataccagagctgtttggaaaatgttagtgctaaaccaacgtgggtgggatttagccaatttagtctccttttgagtttgggatggtagaggagcagtgtaatcttgggtctttatttaaaatgtttgatgacccctttaactattttggctgttgtcaaatgacaaacatcttgtttatcagctgttttaaaaatagcactttgactttttggtttttcccttaggctcgtttctacttgtccttttaaagaattaaattctgaaatatacaaaaattgatataatttaaagaattaacacatctggtgtcctcactgaggttaagattgatagattgttaccagtactccacctcaacaaatccaacctgaaatatggcacaaaatattacgtttcccaatatgttattctgatattattaagtctttgtgaccttgcaaatcacacgtttaatattttgtttctgtatgttaactaaggtataaggaataatgttcagtgaatccttagatgctacctatgatttataatttaaaagtacaaagtatttttggtcataaaatgtaatctcatatggaagtaaatattcctttctagtttgggaaagaaagtattaatgggtgaattctaaaaatgccaattctgcacttgatgttaacttgaagatatacataacgtgcagaacactctagtgctttacatagtttcaagcatttccatttttaagttaaatataccatatttttacatttttttggtttacatttctaactttttgttttcccccctccttccctcaaatgttttgatgattctccagaaccaacgaaacgtatgctttccttccaagggttagctgagttggcacatcgagaatatcaggcaggagattttgaggcagctgagagacactgcatgcagctctggagacaagagccagacaatactggtgtgcttttattactttcatctatacacttccagtgtcgaaggctggacaggtaggagatgttggggtacctgctcgtgattgctgcctctgggtgctgagcttgaaaaatgatacttaaatatttgaacttgaaatttttccagtaccgggtttcaactgagccgccaacgcacatctgctctcttgcccacttgtgacatgcccagctgccagtttttctccttccttttgtggtggtggttatattatcagtatgactagggaccttttctagatttttctatctgggtctgctgctttctagactctgttcaggtaataaattattatcaatgtatatctgagcatccagtctccctttcatgagacctgtcagtttgagggactgaaatgctcagaaatgatggttatgtatgctgtggcaggagatgcatacctggagtttcattaagttgttatggtgtgttgcttttagttaatcagttgaattaatcaaaagggaagaatttgtgttcctgtccttttaataattgttattctggcatatttagacacttaaagatgtgcatcattaaatcttaaatggaaaatgagactcgtgcacatagctttccattttttatcttaatgatcatgatttccaatgtggtgggtttttttttttttttttttttttgagacggagtttcgctcttgttgcctaggctggagtgcagcggcgccatctcggctcactgcaactctgcctcctgggttcaagcgattcttctgcctcagcctcctgagtagctgggattacaggcatgtgccaccacgcccagctaatttttgtatttttagtagaaacggggtttctccatgttggtcaggctggtctcaaactcccgacctcaggtgatctgctcgcattggcttcccaaagtgctgggattacagacatcagccaccgcgcccggccaatgtggtagttttgtaacaagactctagttagaatttttttcttttttttgaggtggagttttgctcttgtcacccaggctggagtgcagtggcgcaatctcagctcactgcaacctccgcctcccgggttcaagcgattctcctgcctcagcctcctgagtagttgagattacaggcgcctgccatcatgcgtggctaaatttttgtatttttagtggagacggggtttcaccatattggccaggctggtctcgaacacctgacctcaggtgatcctcccgcctcagcctcccaaagtgctgggattacaggcgtgagccactgcgcccagccaataattttatttaataagaaaaagcttttcattttgaaatctagaatacctccacttttgaaaatctttgatttcccaaatttttcttgctatgcactagttaaaagataaggtattatataatgagcaatagcacagatctcaaactgtttgagttgcatatgggccataccttcttttctgtaacgtgcatacccatgcattaacacttgtcgccttttccagatctgctcactttagcactctggcaattaaacagaacccccttctggcagaagcttattcgaatttggggaatgtgtacaaggaaagagggcagttgcaggaggcaattgagcattatcgacatgcattgcgtctcaaacctgatttcatcgatggttatattaacctggcagccgccttggtagcagcgggtgacatggaaggggcagtacaagcttacgtctctgctcttcagtacaatcctgtgagtaaaattttaatggttactttcccttcctagaaacccagaaagaaacatagtgtgatatttcagacactaaagtaggttttcacatggaataaagtcaaaagtttagaaatgcgtatgtacttaaaatggtgaaattgcttttagctggtctgcgttatgctaaaatatttgttttatttagcaagttatattaaaaagcataaaaattcttattgcaagaaactacatcaattttaaaataacacgtataaagtacagagcacatgacttttttctgttgtgattaactttggataaattctgataagtgtttgcaagttaattgattttcaaggaatggtggataatgggtcttaatctgtaaaatctgtaatataaaatagtttgaaacaagatgccactatatggaatgatgtgaaaattaggctacaaaatttcttcctcaatatcctattctcttctctttcaatgtaattagaagtcatagaatttagatgatactacttatactgggcttttgattagtttaaaaattttttttgtctaagttgcagatgtgttctgggaacaagtaaggctcagctggcagaccttaaattcatactgtgtttaggatctccgaaagatacaaaaacataagactttccctcagaacttatgatgtagttagagaaagtagatgtaaacttaagaggtaaatgatagtggggttatagaaatctagaaaaagttcccaagttacattgtcaacattcagttttgtaaccaacctggaaatggctaatagtttattttagctaccgtttactgaaagtctgtgtggtcagacactctactgacagctttaaatgattggtcttagttaatcctcacaataattcttcaaaatagatgatattatttctgatttttatcaattaaaaaaaaaccatggactcagggttatgtggctttcatggggacaaagctagtaatgaagtattaatagatattaaaacctgagtctgaatatcctcaaagcaaatgttgaagtgaaaagaaataaaagtgggatgatgtgcccagagggcactcacccatcaaagcagcccttgagcacagttaaaaaaccattgatcatattttgctgtcccttattttttggttaggaaactgaaactaaatttagtagtttgttcaaagatccgaagtcacttctcatgatgatatctattaacaatttttgtagttaattttgagagtttaaacgtgtcctttttagaaaatccattgctttaaaaaacatttttttttaagagatggagtcttgctatgttgcccaggctgtactcatactcctgggctcaagcagtcctcctgcctcagcctccagagtagctgggactataggtgtgtgccaccacacccagcttctaaatttattggtttttgaatggatacagtgaatacctccagttagcactcttggaggtagcaaatgaaactaatttcttgtctatcttttctgagacactcagtgtatatatatgtacatttgagtgactttttgtaagttttattgcaatactaatctagctagttacccacataaagttttagggagcctggtatgtactgccactgggccttggaccccatttttcccagggctcagggctcacctctgtacccagttgtcacagtaagttctttacctttattaatcaggttgattctactcttattgatagtgtaccctcttacacagctgtggttcttgccatctttaactctaatgcccttggctcaggtttccagttgcctatgtgtgtagcagagcagtcattgctactgatagtgtacccaagcagtaggactatgctgagaaggaaaactaaagtgagagatggaaccttgctatttaatatctaacctgctgttagcaggaagcttattgttgtaccctaccaaggaagtgagatagtactaaaataaatcatgggattacctcttgaatgcattgaatggccatgtgttgtcatctggttctatgtttcttctagtggaaggtgctgctttacttatttattactgtttttaccaataagaatgaaaatgggattgtgcaagttcccttgatttcaactaaacatgccatagtgatcatattttaatttatttcaggatatctaaagtcttaactagatttaaacttactgtcaaatgcccgttgtcagttcagtactggtagaaccttgcgtctgttccttatgttcttgttgacagtgcttaaatgcatcattagtacattgtgtacctgaatcatagatgtagttggcattctatgaaaaatggttaaagaaactcaggttattttggttttgcagcagtgaaagagtagaatgttaaggtggtatctttggtaaccttggtaactgttttcaacagtgtttatctttttaagttaatagatatgtcaatttagaagttgttctctgtgacttgattatagaaattatctttgagacagctatctcatttttttcattctgaattatcttctataattataatgttgggtgttttcataattttggtaagattttagtctgtcattttttttttttttttggagacggagttttgcccttgtcacccaggctggagtgcagtggcgcgatctcggctcactgcaacctctgcctcctgggttcaaacgattcttctgcctcagcctccgaagtagctgggatcacaggtgcccaccaccatgcctggctaattttttgtatttttacaaaaacaggctttcaccatgttggccaggcttgtcttgaactcctggcctcaacagatccgcctgcttcagcctcccaaagtgctgggattacaggcgtgaaccactgtgcctggcagtctgttgtatttgaaggtgttattgcagatagcaccttgcagtgacagcacatgttctttgacatggtgactcactagagttgcctatacctggcaattccttttgcaaattatgtataaaattgtcctttctgtaacggctataaccctgagttgttttaaacaggtctgtttctgaatctataggttgttgaattgttgcttgtgctttgattgtggatgcacagaatggagagctgctggcatcctggccatcatgtgtgttcctgtcatgttatttgttacttgttactgctgagcctctcttatacagctgcactgtagaaaaccatatatttcgattcttctctgttctgctttctgttgtatatttcgattcttctctgttctgctttttgttaaggttgctgtgcaaatgtcacatttaaatatgtgagtgcttattgtgcatacaatgctttgttaggaaatggggagatgtgttgtgggggaagttgcgggttataatataagacaggacctgacccgtaaaggaaaaaatggtgtaagttccttggatgataattgttggagagtattaatcagaagatttcctcgagtgggtgcttatgacctaagcattctgtaggaaggtaaatagcaagtagacttccttggctgaagtggtgagtcaagtagtaacaagaagtgaatgtagtagaggagagtcaattggaagatttataaagaatagagaggctgaggagtttgaatttaatttgagaggcaattgagaattactgtaggtttttatcgacctgattttacttcatatgtatgtacgtgtctagatgttgtggctggtgggggtgggaggctctaggaggaaaggcaggaaactgatgcaatcttaggagaaatagttctacaggaaaatcttgctcatttctatctccttttattctcacgattttcccttgtatacagtgccctcctcctttgtctattaaaaccctactcatccttcaaggcccagcttaaacttctcttatgagctcatcatctagttctcttaaaattctttactactgagtgggcatttagtgtgtgccttgtcttttcacatagattgtaagccccttgatgataaggattctatatcttttccctatagttccaggcatgttattgaattgagtaacagtttgcctgaaatgtattaacctgggatggataaaatgggctagttttgagaaatataattgtcaaaaacattttgatttgacagatgaagcagaagtgatgtcttgggttttgaacttttgggagaatggtggtgcccgtgcgttttctagaaattagctgttcatgcacataggtttggatccccaaacaggagaattggatttcaccgttacatttaatatgatgttttagcttttcctaaaactgccttattgattatcttatagtaatcttgaagataatgtatttttaactatgagcattgtgttaacttgatatagtatactgtaatggatctagatgataacaaaatatagatatgctgtcaggacttaagtggcatccatccaagagttcatagaaactagagggtaataaaattacgcaacagctggctactatgtatggccattacagtcattgttggagaattattgcaagacttctgattattatgacttagaaaaggcataggaattggtaaaagtccaaacatgtataaccacaattatcaagtagtagaaggagcttgtatgcatggattggattatgcagtcatggtctgccaaaacaggggtaaccctttcagtttagaaaaaaacataaccatacaaagtagatagcaagattatataattcattacttctgagagtatagtgttaaaaagtgctcaggctaattagaggagaagtcaaataagattaatggtacctttgatgttttgaggattagtattagccttttgtgcttgaagtgatgggagacaaccttactctctagaacatcagtcattgcttgaaggacagtattgagtcagggagatgatggaactgacccagtatggctgaccgtaagattgttgtgtggcagcctgtactttgttggcagactttttggttcacctgatggatgatactatacaaccacaaaagctgtttatcagtagaattaagtgattttggaaaccataatatagttccagagcaaatcatatatatgatgatttttcggcacagttgacaaaatttcactttctaggacttaagtctctttcacccatggaacagtttggaataataagcatacttcctgttgtcattgtttctgcctgttgactggtagatgtgactgacaatggcaaaatagggcaaataaaaatttaagctcactgttgtgatgagtgtctcatccagatgaatggactgatgagtcagatgactagcagcacaattgagctgctaataaaaagcagagtcaactgcttttcattattcacccactcaactctaatggcacttaatactaatgagtaatctagtggcaggtgccaggattgaggctttggttggataattcttctgcccgatagtgaaatgaagtcagagtttctaaatatgcctgacagcctaagggtaggtttgctcataggtagtcatttgctaaatatgtttttttccactctggaaaattttcggtccttgatacagaagtgattcatcataaccttgaataacatagttggacaaatatttgagatgtgtgtgtgtgtgtgtgtgtgtgtgtgtgtgtatatatatatatatatatatatatatttccttttttttttgagacaagagtctctgtcgcccaggctggagtgcagtggcacgatctcggctaactgcagtcactgcctcctgggttcaaccgattctcctgcttaagcctcccaaatagctgggattacaggcacacgccaccatgcccagctaatttcatatttttagtagagatggggtttcgccatgttggccaggttgatctcgaactcctgacctcaggtgatcctcccacctcgccctcccaaagtgtggggattacaggcatgagccaccacgcccggcctgatatatatatttttaatgtgactgacatttggggaggagagaaagaagctctagtaaggtcccatagtattttatgtgaagctaatggggccaaatgcatttcaacattcagaatttttaggatattagaaagataataccatatataaaacacccagaaaagtctggagcagcaccttttagtaaaacacatttatatttctgcagcaaaagacagtattcatactaagtgagataattaaattctataaatagtttcatgtcacctcaagtcaggtttaaccctcaaatgagtttttggaactttttgggatttggaattgtggatgaggggttgtggtcctgcactgaagtttgtcaggcagtatctttagttgatagaataaaatggcagggagctagatgcttaaaagtgatttcaagatatttttaattgacaaagcagagtataattaatgacagcgtctctgggtttctaggatttgtactgtgttcgcagtgacctggggaacctgctcaaagccctgggtcgcttggaagaagccaaggtaggtgtttgatagaacacatttaaacatcagtattatgaaaacttgtactttttgccaagtcttcaactcttcattgagctatcttcacaaaacagtcctttgaaactgaggaaaactgacggcacgaatcgcctcagaatagagcagggccaggctttggcatatctgttctaaatctgggggtaaagcaagaacctgaacattttggagcctttctgctgagctagaccatctttataacactgggctccgtcatgatcttatgtgggaataaataacattccttcaaatctgaggcttgcctgctggtgacaagcagagcgcctgtgatttggctcaagactcctatatgatgcaggtgccattgaaaatgctgctcttctaagtcctttgtggcttgtaagtggagaagaatttcatccaaatgttaccctgtaatactggcatttaaaattcttatttaaccttcctcccttcatcttcctcaccctttttacagtggaagaaaggctgttaaaatgattacaaattaataattggaacatcctgtcccttgtccccactcccttcccaagttcctttttcctcttttccaatcctagttgtctaccttcttttcttcctcatttccttcttttattcctccccaccccaaccccttaaaaaaaaggtcagaaggacaaagctggtttgtttgggaaatggactgatcgaaagaaaacttgccaaagtggaaaggtggcttttagcattctgtgtttccaaataatgaatttgaacaccaggttgggttaattaaagcttttggtataatttaaaattaaatttataaatgcagttgtcttgttacaagccaccttacgcaaccgcgctgcaggggtgaggagtggggagaaaccagaatgcttctgaaactcccacctgttgctctgagccccacgcgcatgctaatgcgtggagtgtatgcgcagagtagctgtctgtttgactgcttcatccagggagggagaaggcttttcagcaccatctaatgttttaaaaggcactagttttaagtgcacagctcataaattctgctgacattttggattaaccttatgtaggttgccagctaatgaattgtaattgatttcaatcttagctgataaatctaattggtaatttatagaacaaatatttgataagctcctattaattgtcaccccaccaagcggacagctaacatgaattgcacttcactgcagctttagagatcggtttaggctgagacattgcgcctgccttaggttgctgacttctttatttcagagctctggagacacctagtttgaaaaatgttattctgtttttttgtgagaacttagtaaacaagaaaatactcttgagtgaaatgcaatgtatttcttttgtaatcagtgcatttgaaaattcaagccagcatattcctagtagatggaagcaaaattaagttgtctttgtagaaaatgaagagcctttcttccagcaaaaatccctgctgtatgcaatagccctgattaaccctctcccttctgcatgtttcccatattacagacttgagactgtcctcattcccatatgtaatagacatccaaagaatttcaattgctttgttgaacttttactaatgatcttgtttttattttctctcttgtttttggtttttcaccattgatattgtatttagaaggtttcaggtgggtgaaacctcctattccatgcgtaaggtgcctcgctgaagggagctcgaggcctggatctagggcagacacacaacctcctcctcctcttccagcaaggaacgcaccgaaaagtcacatgatgagaaatatggtaacgggtttgtaactgccacagcaaaacaatttgcctccatgcctgaatcttctgtcttgtggcttcagaaacagcttaaaataattttatttacaagcaagttatgtaagagaatgttttatactatagccacaattctgtcaaagataagtaaaagttaattgatattaaaaattattagagataatttacttagtaaaagcttctaactcttcttgttgttcattttttttccttttttcttctttgtttggattgcagcattctgctcttctgatgatgcgctgtgaccctgcagtagcgcaaaggctgcgcagcgttaatgcgcattgcgtgcgaatgaacccctgtgaacggttgactagatgagtaatctgattgactggctccctcagtcctattctgtagcctttttggataaaattgggttttaacatacctcgagtccaactaatctcattaaacaaatattctccatgggcctgtctagtagattaatggatctggttggccgtttgctgcgtctaggggtgttctatgtagcgcagcagttcgcagcgattgcgcagtgcgatgctgttaggttgcgcaagcgatgtttgcgctcgcattacagggacctcaacctaggtgcaatcctgtcatgtgaggtttcagcttcagtcctccttgggagacggggcattgtgagaatgtaacttaaagcctggctttatgatatcctacttggcagaaagacatttttctcctcagtagcatagttttgatgttagtgaggaacattgttgaagagcagcatttcccaaaatgtgtttcatagtattctaataaaatgcccaatgaaagaagagttccatggtcaactaagttcagggaaccctgttacactattaaaggcttagggaagtccagtaaagaaacctattttccgaatttatttgatcatgaactcctttttttttcagccatacctcttaacacctcatagaacacactttgggaaacagtgggggtaggaaaactcggcctcaagttgcgccctctaggtagcacttgaaaacatgacaagggcccgtagttgtttggataagagaactccagcatagagccttatagcaactgacttcccagttaagtcccagtgtaagggttggtctttggttggcagaactgaacatggtggtttgcacttgggttctggtggcgcaggcgcaggagcagccagctgtggcagcgcattagttttggcgcaagcgagcctatgctgcagggtcacttttggctggtcagagaaggaataatgatatcaccttcttccccccctccccccaatcttttttttttccctttacaaattttcccctttccctttacctcctttccctcccatcttctttcattaacccctcctaaggcatgttatttgaaagcaattgagacgcaaccgaactttgcagtagcttggagtaatcttggctgtgttttcaatgcacaaggggaaatttggcttgcaattcatcactttgaaaaggttagtcattaaattaataattggtatttttgaagtgcttacgcatgtagtgtttttactagaactaaataataggtcttagccagtgacattatatctttgtttctctggcggtataggcagtatgtgacttacagagaacaatgatttgacttaaatattttttcaacttgacaatggtgcaaaagacatacatattcaatagaaatcatacttcgagtacccatgcagccattctggttttccctttcagtacaagattcagtaaattacatgagatgcccaacacactattataaaataagaaggaggctgaggcaggaggatcacttgagcccaggagtttgaggctgcagtgagctgtgaccatgccactgtacttagcctgagtgacagagtgagaccctgtctctaagattaaaaaataaaaaggtaccataaagacacatgcatgcgtatgttcatcacttcacaatagcaaagacatggaatcaacctagatgcccatcaacggtgggctaaagaaaatgtacatatatgccatggaataccatgtagccatcaaaaatgaaatcatgtcctttgcagcaacatggatgcagctggagtttattatcttaagtgaattaatgcaggaacagaaaaccaaatattgcatgttctcacaagtgggagctaagcattgggtacacatggacacaaaggggaacaatagacagtggggcttacttgagggtggagggtgggaggagtgtgagggtacaaaaactacttaccaggtactatgttcactacctgagtgacaaaatcatttgtataccaaattgctgtgacacaatttacccatttaacaaacctgcatgtgtaccccaaacctaaaataaatgttggaattaaaataaaactcatattaaagttttaaaaaagggctttgtattaggtaattttacctaaacgtaggctaatgtgttctgagcatgtttaaaatgagactagccgggccaggtgtgatagctcacatctgtgatcccagtgctttgggaggccgaggcgggcagattgcttgagcttaggagttgaaaaccagcctggacaacataacgagaccctgtctctacaaaaatgagctgggtgtggcagtgtgctcctgtagtcccagccatttgtagggctgaggtgggaggattgcttgggcctgggagctggaggttgcagtgaaccaagattgcaccactgcactccagcctgggtgacaaagtgaggccctgtctcaaaaaaaaaaaaaaaaaaaaaaaaaaaattaggctaagctaggatgttcactaagtgttacagatgcattttgacttaagatattttcaacttatgatgggtgtatcgggacataaccccatcctaagttgagaaacatctttattgcagcatttgctggatagtacatggtatggcagtttccaattttgtgttgaaaatgtactttaggctgcgtgctgtggctcacacctgtaatccagcactttgcgaggctgaggtgggaggattgcttgagcctagtagtttgagaccagcctgggcaacatagtaagaccctgtctctacaaaaaatacaaaaagtagctatgcgtggtgacacacacacagctactggggaggctgagatgggagtttgagtctgcagtgagctgtgatcatgcaactgcactccagcctgggtgatggtgtgagaccctgtctcaaaaaaaatgtactttagtaattaaaaatgatttttataaatgggctttaaagaagcagggcttcttggtgaaaaggcagattccaggtctggggcaggaatttaaaaaaaatttttggaatgtcttgtcataccagaaagcaaggaagctttgacatattactttgcatcaaaaggactcaggaagcaacttgaggaggctcccactggctaaagatgcagacttgagcatcaataggaataataactgaaatatactaaaacttatcaagtatgtttaaatctatgagcttataattatgctaaaaaattcattggtcatctttggaagacttacaaggaacaattaatttggaaaactggttaaaaagagaagaataaagtttttgaggaacctctatactagttcccaaaatggctgtgccaatttatacttcccccatcagttacaaggcttccctttctttacatcctcactaacacttgttattattcgtccttttgatgatagctatactaacaagtgtgagatgctaactcatggttttattttgcatttccttgatgattagtgatgttgagcatattttttatatatctgcttgccatttgttttttttgttttgttttgttttgtttttttgagatgaggtcctgctctgttgcccaggctggagtgcagtggcgccataatagctcactgcaacctcgaactcctgggctcaagtgatcctcccaccttggtctccccaagtgataggattacagacatgagccactgtgtctggcctgtacctcttcttttgagaaatgtctgtttagatcctttgcccgtattttaatgggcgtatttgctactgagttgagcttcttaaccctttatcagttgtatagtttgcaaatgctttctcctaatccggggttgcctattcactctgtgtaattgtttcctttactgtgcagaagctttttagtttgatgcaatctcattgcatatttttgcttttgttatctgtgctttatgctaagtgaaataaaccaggcacagaaagtagatactgtattatgtcacctatttgtggaatctaaaaaagttgatttcatagaaacagagagtagaagggtggtgaggcttgggggggtggaggagggatggggaaaagggagatattgatcaaagtttcagttagactggaggaataagttttagtgatctgtagtactgcatggtgaccacagttgttaataatgtatatttcaaaattgcaaaaagaataggtttttaatgtcctcaaccacaaaaaaataagttagtgaggtgatggctatgttaattagcttggttgactctttctaaaatgtatacgtggatcaaaccatcacattgtatgccataaatatacacaattatatatcaactttaaatttaaaaattaaaagaataaagcatatgtcttccctctcctgtatgaactagaccatagagtaacccaatagttgatgggggaagcttttctttataaaagcattctagctaataagtgaagaaggaatgatagaatatcaccattaatagtgaaagcatttattaggccgggcttggtggctcacgcctgtaatcccagcacttcgggaggccatggcaggtggatcacctgaggtcaggagttcgagaccagcctggccaacatggcaaaaccccatctctactaaaaatacaaaaattagccaggcatggtagcaggagcctgtaatcccagctactcaggaggcggaggcaggagaatctcttgaacctgggaggtggaggttgcagtgagctgagattgcaccattgcattccagcctgggcgacagagcaagactccatctcaaaaaataaaaaaaaagttaaagcatttatcagtgacaaaaacgagatacaatcaggtattaagtaccttgtggtaaacaatttcaccagtgaagtagtcttgcacaaagagaaaagaacacaaacctaattaagcttctggttctacctaccaatttacaggagatatacacgggacaggaaacatgttaaaggacaccatagggatgcagtcagcaaaattcaaactttgggaagctgaccttcttacttcaacaaattgtaataaaaatggagatttaaattaaacttttaggccgggtgcggtagctcacccctgtaatctcagcactttgggaggctgaggcgagtggatcacttgaggtcaggagttcgagaccagcctggccaacatggtgcaaccccatctctactcaaaatacaaaagaaaaaaaaaacagctgggtgcggtgcggtggcgtgtgcctgtaatcccagctacttaggaggctgaggcaggagaattgcctgaacccgggaggaggaggaggttgcggtgagccaagatcgtgccactgcactccagcctgggcgacagagtgagtgacactccgtctccaaaaataaataatgagtgacactccgtctccaaaaataaataaataaacacacaaacttttaaaggattaaaagaaactcagagaggcacattgacaaattatatctgtcttatttggatcctgactcagttaatgagaaattggagcactgaatggatttttttttttttttttttgagacagggtctcacttgatcgcccaggctgaagtgcagtggtgtgatcttggctcactgcaaccttcacctcccaggctcaagggattcacttgcctcagcctccggagtaggtgggactacaggcgtgcgccatcatgcccggctagctttttgtatttttagtggagacagggctttgccatgttcccaggtcttgaactcctgagctcaggcagtctgcctgcctcggcctctcaaagtgctaggattacaggcatgagccaccacgcccagctgatatttaatatgaaggaattgctgtcttttttttttttttttctgttaggtatgataatggtattgtggttaggttgggaggaaaaagcattcttgtgttgtccctgagatacatattgaagtatttttgaatggaatatgtctgggctttgcttcaaaatgatctgcgaagagggggagtaagttggagtatagattaaataagatcaaacatgatatatgaattgttgaaaatggataatggagacttggacgtaatgttcctctgcttttgtatatgtttaaacatttctattataaagtttaaaagttcttggggtggacatctatcactatagacaatctaaattaaatgcatcatgccgtgtttactacagagttgtcatctaagtgaagatgaagtgttctttctttatttttttttgagacagagtctcgctctgtcgcccaggctggagtgcagtggcacgatctcagctcactgcagcttccgcctcccaggttcaagcaattctcctgcctcggcctcctgagtagctgggattacaggcgtgagccaccatgcccggctaattttttgtattttttagtagagacggggtttcgccatgttggccagggtggtcttgaactccgggcctcaagtgatctgcccgccttggcctcccaaagtgctgggattacaggcatgagccaccgcgcccagcagatgaagtgttctttatcagtctttaataaacatacaattactcagtgtcttaatcaaataccttttcggtgtgtgtgtttttttttgtttttgttttttgtttttggagacacagtcttgctctgttgcccaggctggagtgcagtggcgcaatcttggctcactgtaacctctgcctctcgggttcaagccattctcatgcctcagcctctcgagtagctgggactacagttgcacatcaccatgcccaggtaatttttgtatttttagtagagatggggtttcaccatgttggccaggctggtcttgaactcctgacctcaagtggtccgacctcggcctcccaaagtactgggattacatgtgtgagccactgcacctggccggtgttttgattgaagtttgcatttaccatgctgtaggagtatctttcttgatcagtgtttctcttccttggttatacatgaggagcttataaaagatctgaatatcctgaccctatcttcagagggtctgatttaactggcctgtgatagagcctcggtaccttttctaggaggtaaattcttaatatattgccaaggttgagaatcactgctctagacaacatgacagctatttggatgacaccgtgtaatggttagtatttccttgaagtattgtatcttactgtgtatacagctctaattgcatgtcttaataaggtcttgctgagcatgtagtttgtatgcatacaactgaggtatatatttaccttttcagtaagagtttgctgcctcttttgccactatttttcagaagagttagactggttaattgtgaataaaatagggccaatcattagaagtgttcagtttaatgaaattggagcaggcccaatatttaatttttctcttcaggacccattttgctcatgagaacatgaggcatgagaagttattttgataatgttgaggtttgtaaaaataatgagaagacactaaataaataattggtttattaaaatgaaatgtaattgaaagtctgcaaggaccttggagtaaaaaaattgtaattgggaagttgatctggtgaaatcctaacttgctctgagtaactaactgtcttgaaatttttaggctgtcacccttgacccaaactttctggatgcttatatcaatttaggaaatgtcttgaaagaggcacgcatttttgacaggtgagagaatgttctgtttaataaattttcttgaatctttgttgtattgacacttgacagtttggaccgaaatgatgacaatagtccattgaagcatatttgccttttctagcacgttgttctctgcccatgtcctgtgttgccttttatttatagatgttgttcgttgcctgactttttgttgttggctttcctcaaagccgttatccttctcattgggagtttagttgaaatttttttattgttaaaatggttgaaatgattctttcttagactaaattattcctttgacctgattttagaaagcaaagtaaatccagacttttaaattttttgttgttgtcaaaggaaagcctattttttatgtgggagtagaaaacttcccagtgtaaagacacattttataaaggtgtgattttggtttgaggttaaaatataatacagcatgaattatgtaattaattacaattacttgatatattttgatatagtacagcttgaatgagttgtaacaaaccatccattaagcatgagttacattttgtgtgtgtgtgtgtgtgtgtgtgtgtgtgtgtgtgtgtgtgtgtttatttacagagctgtggcagcttatcttcgtgccctaagtttgagtccaaatcacgcagtggtgcacggcaacctggcttgtgtatactatgagcaaggcctgatagatctggcaatagacacctacaggcgggctatcgaactacaaccacatttccctgatgcttactgcaacctagccaatgctctcaaagagaagggcagtgtaaggatttttactcattctatttgttatctggtaggattaagagtcttttctggccaggtgtggtggctcacacttgtaatgccagcacttcgggaggccgagatcagaggattgcttgagcttagggtttcgagaccagcctaggcaacatgacgaaaccctgtctctacaagaaatacaaaaattagccgggcatggtggcacacacctgtagtcccagctccttgggaggccgaggtgggaggatcgattgagtcctgggaggttgaggctgcagtgggccatgattgtgccacagcactccagcctgggcaacagagcacgagactctgtctcaaaaaaatcacaaaaaccaaaaaacagcaaaaaaggaaagtcttttgtacaagtattgagatggtatattggtttactttagagttttggttgagggtaagaataccaaaaaatatcaattttctgtagcattaccagccattaggcttaattaaacaattattagaacagtatcagtggaggctttatgattctgtacagtttttgaagactttgttttgttttctaggttgctgaagcagaagattgttataatacagctctccgtctgtgtcccacccatgcagactctctgaataacctagccaatatcaaacgagaacagggaaacattgaagaggcagttcgcttgtatcgtaaagcattagaagtatgtgagggtggtgtagctgggcatttagcatgatagtagagggaaagcagttaagtttaccatcatccacctcttttgtaaaaatagtggttgaatcatttacaagaggattattcattgaagtaacaattgagtacctaaggtatgatgtgaggccctatgtgacattccagaaaatccctgcattcagagttcattattctgtggataagatctgtacaaaaaaattaaacattaacccacagttcaagagaagttacttataagtatataatcttagtgcctgatggctgtatggtagtggttagtagatgtggtaggagtttagcagagagagatttcagtggtgttggagtggtcaggataaactaagaggagaagaggtgtacctcattatgtttgaaacaagaagaggtataaaatagaggaagaggtagaatttgagaatgaatagacttggaaaaagtagagacaaatttaggggtttaaatgtacttgagcaaagttcttaaggttgtggtgaattattactgctgctttagcaatttttttaacctcagttctgatcttgactctttataggtcttcccagagtttgctgctgcccattcaaatttagcaagtgtactgcagcagcagggaaaactgcaggaagctctgatgcattataaggaggctattcggtaagagacactaacagccttatttttaaaattatttttaattttaaaatgtttgacattcggcacattgcagatgctaaaatggctttaaataacaacggaataagttagcattacagtgggttaaagatttttgtattggagaaataaaacctttggcttctcttttagaatcagtcctacctttgctgatgcctactctaatatgggaaacactctaaaggagatgcaggatgttcagggagccttgcagtgttatacgcgtgccatccaaattaatcctgcatttgcagatgcacatagcaatctggcttccattcataaggtactactgtttattataatatgtgcagtttaacacctaaaatttaacttttggaaattttttaccatcctgctttatttattttccaggattcagggaatattccagaagccatagcttcttaccgcacggctctgaaacttaagcctgattttcctgatgcttattgtaacttggctcattgcctgcaggtaaagaataacaggccagtaattggctctcagtgttgtaatagctttttaattgttgtatgccataagaatccaagcctgactggaaatagtatttttaataggctatctgacattactttaggccaaagacatatcaaatattaaatcctgggataggactttctggataataacttgtttttgctttctctagattgtctgtgattggacagactatgatgagcgaatgaagaagttggtcagtattgtggctgaccagttagagaagaataggttgccttctgtgcatcctcatcatagtatgctatatcctctttctcatggcttcaggaaggctattgctgagaggcacggcaacctgtgcttagataaggtgtgattcttttgttttaatctttttgttgtaaactaaaacacaaatacagaaaactgtaaaaatcaaatctgtggcttaatgaacgattataaagtgaacaccacccaagtcgagaaataaaacttttgtgggccacttcagaaccccttccattgtgtcctgagtataaccccctgcacttcataagacggaatccttaactccttcacttaaacactaatgatagactgatggtacttttctttaagttgaaagccataatttttttttcttttatcttttttttcttttttttttgagacggggtcttgctctgtcgcccaggctggagtgcagcggtgtgatcatggcttactgcagccttgacctccccgagcttaggtgatcctcccacctcagcctcctgagtagctgggactacaggcgcatgccaccacacctaattttgtgttttttgtagagatggggttttgctatgttgcccaagctggtcttgaacctctgggctcaagtgatctgcccacctcgcccggccaaagtgctgggattacgggtgtgagccaccgcgcctggccagccatcatgtttaatggagtgcttggtattttgtattcacctttgagaattagagcagtacttgatcaaggattggatcaaaggttggataatagattggagtgctaggtgtactttttttttttttttgagacatagtgtctctgtcatccagactgcagtgcagtggtgccatctcagctcattgcaacctctgtctcccaggttaaagtgatgctcgtgccttagcttcccgaggagctaggattacaggcgtgtgccaccacgcctggctaatttttgtatttttagtagagatagggctttgccatgctaggctggtctcgaaatcctgacctcaggtgatccacctgcctcagcctcccaaagtgctgggattacaggcatgagccactgcccccagccataattttgtttttagaaaatgtttccctatagaattttctaaaatgcgggaggaaaccatgctctgtccatactgcttcatacctacaagtgttagatttgctcgtacttttttttttttttttttttttttttgagacagggtcttgctctgttgcccaggctggagtgcagtggcacaaccttggcttactgcaacctccacctcccaggtttgggcgattctcccacctcagcctcccaggtagctgggactacaagcatgtgccaccacgcccagctaattttttttttttttttttttttagtagagattggatttcactgtgttggccaggctggtctcgaactcctgacctcaagcgatccacctccttggcttcccagagcgctgggatttgctcatgctttatagatctcaggagcaggtcaaagtatgaagcatattacataaagtgctgaggttgcatctctctggcaatattaactaaatgctctgggcagacctttttcaacaggtgtgaggtataggtttggtgtgtttttcgtcagttttcctagatgaaaatttatgtagattttactaacaagcattggattctgttgatagattaatgttcttcataaaccaccatatgaacatccaaaagacttgaagctcagtgatggtcggctgcgtgtaggatatgtgagttccgactttgggaatcatcctacttctcaccttatgcagtctattccaggcatgcacaatcctgataaatttgaggtaagactagtgtttctctagaatcactatttgtttaaaaaagaaaaaacccacaatgttggcttgtcaccatgaggcatggaaacctagtgtcttttggaaagatttgagccagaaagatttgagccaatgttattaaatatgccatgtgctgcctttcaggtgttctgttatgccctgagcccagacgatggcacaaacttccgagtgaaggtgatggcagaagccaatcatttcattgatctttctcaggtagatgaaactctcatactttaactttttattttgagcaagtttaaataaaactattagcatatagtttatttttgatgataagtattaaatgctgtagtaccacatataggcaattgtgttgatcttatttccttgatcatttgtgttaatcagcatggctttttccgaggttgcaaatgaaactgaaatgtgttatgcttgtgcttggacatacaacttttctggagattgagccagcacttgtgacagaagatacttaataaatattgacaaaatggtgacttgggagaagaataaataggtcttttaaagaaaatagggccgggtgtggtgactcatgcctgtaatcccagcactttgggaggtcgaggcgggcagatcatgaggtcaggagatgagaccatcttgactaacatggtgaaaccccgtctctactaaaatacaaaaaattagctgggtgtggtggtgcattcctgtaatccccgctacttgggaggctgaggcaggggaatcgcttgaacccgggaggcggaggttgcagtaagctgagatcgcaccactgcactccagcctggcaagagcaagactctgtctcaaaaaaaaaaaaaaaaaaagaaaaaatagatgaactatggttcatcagtttccatgggcctttgcctttggttatctttatttctgtacttgttctattgttgtatcctctaggagagtaagttttatgataaattaagattgaaacctatgggggaattaatttgctgagtatgtaggctacagtgttttctttgtagactctattagccactttgactaatagagataatatactctttatatgtgtgtgtacacacatgtatgtatgtacatacacatgcaggtatgatatgtatgcatatatgtataaatatacatgtgtatgtgtatatatatatgaatcactgaagtcctagcagaaaggttttgagacttctcatttaataaatagtatgtactaacaaattttgcagtatagctgttgcttcaaaagtatacctggccatttttgatacacagttggactctttgtgaccaagaagcatacctctttctacctctgagacctttagggactcactgctgattgattgcagtctaaactcttcagcatagcttttgtaacctttctctgtttgccacaactttgtcaatactttaactcaccattcaacatcttgtataccctatactaaatagaagtgaattactcaccctttttttttttgagatggagttttgctcttgtcgcccagggtggaatgcagtggcacgatctcggctcactgcaatctttgcctcccaggttcaagcgattctcttgcctcagcctcccgagtagctgggattacaggcgcctgccaccacacccggctaatttttgtatttttagtagagacggggtctcaccacattggccaggctggtctcgaactcctgacctcaggtgatccacctgcctcggcctcccaaagggctgggattacagatgtgagccaccatgcccggcctactcaccattttctaaactccctcacactttactctagttcttcattcatactgttctctactgagattgtattctgttaaactccttcaaactcctattcgtctttgaagacctattttcaaataccaattctaggctgggtgctgtggctcacgtgtataatcctttgggaggctgaggtggaaggattgcttgagaccatcatgggcaacatagtgagaccccatccctaccaaaaaaaaaaaaaagattacaaatttcaaataccaatttctctgtgatatgttgttattcatacattcaatccagaagtgaactctccttcctttgtgttctgtttctttaatgctttgggcccattttaatgacagtcaaccatatttgtgcccgaattaatgtagttaatttgtgttcttacctcttttccatctgggaatctgaattatgttaaatgccattaaaactaaatgccattaaaactaaatgtcattaaaactaattgatctgagattatacatagtgagttcttattttgtattctgtagattccatgcaatggaaaagcagctgatcgcatccatcaggatggaattcatatccttgtaaatatgaatggctatactaagggcgctcgaaatgagctttttgctctcaggccagctcctattcaggtaaacaaattaacagtcatcacttataacatgtatttggctaagaatacagtgaggtgctgcttttcctccctctggttaactgatatttgaaactgtagggcagacatacttttaatttttttaagttgttgaaatgcacaggacaaaaagagtgtagagcacagtgcttataggtacatgtatatagagacatgcaattcatggttggctatgaacagttactctgtcttgcaaatttgaagaacattttagcattttgaaagttagtctgaaatacaggccgggtatggtggctcacgcctgtaatcccagcactttgggatgcttaggcaggaggatctcttgaaaccaggagtttgagtcctgcctgggcaacaaagcaaaacaccctatctctacaaaaaattaaaaaattagccagagtggtggtgtgcctctgtgatcccagctatttgggaggctaaggtggaaggattgcttaagcttaggagtttgaggctgcagtgaactatcactatgccactgcactccagcccaggtgacagagtaagagtctctcttatagaactgcttcattgctttcggctaaagaagtcagccttgctgtgcgtgtggcaagtgcctgtaatcctggctactctggaggctgaggcagaaggattgcttgagcccaggagtttgaggctgtagtgatctacgattgcaccactgtattccagcctagggaacagagtgagatcctgtctctaaaaaagagttagacctccttgtttatttacttatttaattttggcacctttaggttctttttttaaaaaccactttattgaggtacagttgacatacaaaaagttgtacatatttaatgtatgtgacttgatgaatttagaggtaagtgtactgataaaagttcttaatcagtttttgatctgatttttttaaggcaatgtggctgggataccctgggacgagtggtgcgcttttcatggattatattatcactgatcaggaaacttcgccagctgaagttgctgagcagtattccgagaaattggcttatatgccccacactttttttattggtgatcatgctaatatgttccctcacctgaaggtaggtatgaaacagtgctatggacgaatttcaaagaactgtagctttttatttccttgctattgtctatgtaaatcctaaaagacatgtctgaaactattttttccattagaaaaaagcagtcatcgattttaagtccaatgggcacatttatgacaatcggatagttctgaatggcatcgacctcaaagcatttcttgatagtctaccagatgtgaaaattgtcaaggtcagaacctagtcagtattgtcattgaaataaagttaactgcattcacgatcttttccctaatgatgcatttttttttcagatgaagtgtcctgatggaggagacaatgcagatagcagtaacacagctcttaatatgcctgttattcctatgaatactattgcagaagcagttattgaaatgattaaccgaggacagattcaaataacaattaatggattcagtattagcaatggactggcaactactcaggtgagaagataataatacaccattatatgtcccgccaagtatgcatttatttcccttaacctcatgataactctaggaggcaagtatcattatcaccatttttatggatgaggaaatgagtttagtggaaacgtgcatcttatataggtgtgtctgattttaaaagtctcctggattcagccagcagattattacttactgagaaactgctgtgttaatatcttgttattccctgcctgtggctttactctcttacatgatgaactatgtgtagattccatacagtttcttgcttctgtaaacccatcctttccctagctaaatcctactcaaacgttcagttcagacagctcttccaggaatccatttccgtacttaccgctaacgtctccatccctacttcattggtacctgtcttttatacctctgtaatgtttggaatatatcattacatacagcacaacagatgataattccctgtgtttgtatcctctactgaattgaattactgagcccatgtcttaatcacagtaatgtagtatgtactcaataaataatatattgcatgcttaaataagtagctctatcaagagagtgtaaggcaaagatctctgacctctagaggtcatagatgtccttgcacacctggaatagttatataacaatgtaaggcagtactcaaatgtgtgagaattacaagatgaaactaattatgtatgtagaagctcagaaacggtgaggatcaatgtgaagagagacactcttgggagaagagaccatttggaggagtaatttagccttgaagagtagatgtgatttagaaaaggacatttcattcatgaagaaaagagtgatcaaaagctgttggaacgttaataggattttgagaccttttttggttagagcaagaagtttcatagtgcagtctcccccaacttaagataatgctgcaacggtttgccttttcttaaacttcatatatcatgaatattttctcatggttcagtgacttttaatggttataccattttggatcttatggattgaaatattggactccttttgcctttaaatataaccatcatttttttcttgttctagatcaacaataaggctgcaactggagaggaggttccccgtaccattattgtaaccacccgttctcagtacgggttaccagaagatgccatcgtatactgtaactttaatcagttgtataaaattgacccttctactttgcagatgtgggcaaacgtgagtatgcaagtatgttagagactaataaagattttgtatctagagcttcttgctgaagatggtccttccactcccatttaggagctgttctgctcatttctttttaaaatttttttttggctgggtgcagtggctcacgcctgtaatcccagcactttgggaggctgatgcgggtggatcacctgaggtcaggagcttgagaccagcctgaccaacattgcaaaaccccgtctctattaaaaataccaaaaaattagccggacatggtggtaggcacctgtaatcccagctactcgggaggctgagacaggagaatcacttgaacccgggaggtggaggttgcagtgagccaagatcgtgccattgcactccagactgggcaacaagagcaaacctccatctcaaaaaaattttttttaaaaatactttttaaaaaatttattttatttatttacatgtgactcaactgggttatgccctttttttaaaaaacagaatctcactctgttgttcgggctgcagtgtagtggtgtgatcttggctcatggcaacctttgcatcccaggttcgagttattcttgtgcctcagccttccaagtagctgggattacaggcatgtgccaccatgtctgggtaatttttttttgtatttttagtagacggggtttcaccatgttggctaggctggcctccaactcctggcctcaagagatctgcccgccttggcctcccaaaatgctaggatgacatgtgtgagccaccgcacctggccttgtgctcatttataatcttcaagagatgtagatttttcaggcatttttggtttctccttccagtgtctaaatatgacattgtgaaccttttaaatttacatattcaaagcattcttctgtctaaagggtcagcaaactttttcttaaagggccagatggtaaatattttcagttttgtggtccatgctgtttcacgtctactcagctttgccgttgtagcacgaaaataaccataggcattttgtaaatgatgagtgtggctgtgttccaataaaattttatttatacgaactggcagtatgccagatttggctcacaggctgtgtagttcgccaactcctggtctaaactgatagcatatgctgtcatgtacatccacccatttttaacattcttgtagtattgtagagttataaccaagaaggagtcgatctgtacatatgattatctctcagttcatcagacaagtttgcataagtgggatatcggatagctataactggctcttaatccatagaactaaaataaaatggcatgttttacctttaggtaatctggagataattccttggttaattttttccccgatatctgttttgttttatgtttagaggagaggggaggatagaagagaagaggaagcagatgagtacaggtctactgtccttaatccttacttcctgatgttcacaaaaactctgaaaatcaaaagctttttcataagtgtgtggcaaatttgtttggcagcaaaactacctgaactaacattagggtatatattagtccgttttcatgctgctataaaggactgcccgagactgggtaatttataaaggaaagaggtttaattgactcacagttccgcgtggctatggaggcctcaggaaacttagagttacggcggaaaagggagcaaacatgtccttcttcgcgtgatggcaggaagaagggctgagccaaagcggggaaagccccttataaagccatccgatcttgtgagaactcactctctatcacaagaacagcagcattggaaataactgtccatgattcaattacctaccactgggtccctcccatgacacatggggcttatgggaactgcagttcaagaagagagatttgggtgggggacacagtcaaactatgtcagggtatttgtagtcttttatttatttcatttagtatgactgttcctgcatctcaatgcagaggtactaatatgtttataggaagttgctgctgagggtgttaatgtgatacatacacacacagtactacttttttaaaatctgaaatattctgaatttggaaattcatctgttctcatagctttcagagaaagggattttgaacttacatggtacatagagtagatgtaaatctaaatcaggagttggcaaactatgacctgtgggtcaaatccagcccactgcttgtttttgtaaataaaacttaatggacacatagctatgcctacttgtttacacattgtctgtggctgctttcaagctacagcagaagaactgagcagctatgacagtccatgtggccacagaaccaaaaatatttgctatctggccctttacagaaaagaccgatttatattaggcaaagaaagttaggcaagtgtgaggtaactttcttggttgtatcatagtgggggatttgaaatttggaatgcaaaattcctctctcagcaaaacccactgcctgtgtcaattcccgcaggaggtagctgcctcagattttgcctgtctttctttgaactcactttgaggacagctaagcagaagagggataattcaattgatctcattcatctctgtgaaggaaagacttaagtccacagcaggcttctatctggatggaaagaatagagtgtggtagaattattagagacctctgggagtctttgttgaaaaaggcagatctgctattaataattattcttattttccctattttagattctgaagcgtgttcccaatagtgtactctggctgttgcgttttccagcagtaggagaacctaatattcaacagtatgcacaaaacatgggcctgccccagaaccgtatcattttttcacctgttgctcctaaagaggaacacgtcaggagaggccagctggctgatgtctgcttggacactccactctgtaatgggcacaccacagggatggatgtcctctgggcagggacccccatggtgactatgccaggtaagttgctgataaatcactggaatcttccttgttcctttgaaaatctccgtttgggggagaaacttccaggtgaaattatatgtactaggagcaacattaaaggaaaggtgatagaaagtgagacttgttctcacttcattctcttcatctgcgttgtgtggaggcttaacataaataacattaactctgtgctgttttacgttctcagatgtgcagttgttatcttttgtattacaggagagactcttgcttctcgagttgcagcatcccagctcacttgcttaggttgtcttgagcttattgctaaaaacagacaagaatatgaagacatagctgtgaagctgggaactgatctagaatagtaagtaaacttttccttgaacaaattatttggtaaagtagagagaatatagctgttataaaatgaaaccataaaataagtgtaatataaaataggtgaactagttgtatgccctctggatgggagaagtacttctaaactcttagaaatgaagtgatcaataggttttactatgtaaaaagtaaatggctgaataaaaaatattcaagaaaattaaaaagcaaacgaggggaaaaaatcttttgtctcagataacaaagtattaattacctggcaatgtaaagactgcagaacatttaaattttataatcccctaataggaaagtaaaaatggaaatgaccagacacctcactgaagtggaaatacaaaaggttaacaagaaagtgtttcaggctctgtagtagaaatgccaattaaaatggaattgaggctgggtgtggtggcttatgcctgtaatctcagcactttgggaggcttaggcgggcggatcacttgagatcaggagttcgagaccagcctggccaacatggtgaaaccctgtttctactaaaaatacaaaaattagttgggtgtggtggcatgtgcctgtagtcccagctactcaggaggctgaggcaggagaatcgcttgaacccgggaggcagaggttgcagtgagccgagatcgcaccattgcactccagcctgggtgacatagtgagactctatctcaaaaacaaaaacaaaaacaaaaacaaacaaacaaaaaaaaaaccaaacaaacaaaaaaaaccaaaaaaagaacctgaggtatatccattatttgcctatacaataaaaagattttttgtatcagtgaggtagcattgaggactagcctttttatgcattggtgaataaattggtacagcccttcctcagagtttttagagccttaaaagttagatctttgggccaggtgcggtggctcacgcctgtaatcccagcactttgggagggcgaggtgggcggatcacgaggtcaggagatggagaccatcctggctaacacggtgaaacccccctctactaaaaatacaaaaaattagccaggcatggtggcgggcgtctgtagtcccagctacttgggaggctgaggcaggagaatggcatgagcccaggagacggagcttgcagtgagctgagattgcgtcactgcactccagcctgggcaacagagcgagactctgtctcaaaagaaaaagttagatctttgaccctaatactgtcacttcttcactacggtcggtgtaagaaataaacaaagcataacttaaatgcctgaaaatgagtaggaattagaggggtgatgcttagatgaggaggatttatccatacaataaactagtatttctttcattcttttattttttttgagataggattttactctgttgcctaggttggaatgcagtggggtgatcttggctcactgcagcctccacgtcctgggctcaggtgatcctcccacctcagccacctgagtagcagggaccataggactataggtacatgccaccatgtcccatgcctggctaatttttaaatttttttgtagagattaagtttcaatatgttgcccaggctggatatttcttttttcttttccttttttttttctggacggagtctcgctctgtcgcccaggctggagtgcagtggcgcgatctcggctcactgcaacctccacctcccgggttcaagcaattctcctgcttcagcctcccgagtagctgggactacaggtgcacgccaccacgcccggctaatttttgtatttttagtagagacggggtttcaccatgttggccaggctggtcttgaactcctgacctcgtgatccacccaccttggcctcccaaagtgctgcgattacaggagtgagccactgtgcctggcgttttttttttttttttttttttttttggagacagcctcgcactgttgcccgggctgaagtgcaatggcacgatctcggctcactgcaacctctgcctcccgggttcaagcgattctcctgcctcagcctcctgagtagctgggattacaggcatccaccaccacatccagctaatttttgtatttttagtggaggctggatttcactatgctggccaggctggtctcgaactcctgacctcgtgatccacccaccttggcctcccaaagtgctgggattacaggcataagccaccgtgcctgacgggtatttcttaaatgaggtttttctttcggtataatgggtgatgttttttctttgtgtttttctgagaagtagcatattacataataattaaagaaacatactttttgagtcagactacctgagtgaacccaagctctcctacttatgagttttgagactttgggcaaataaatctctgtgttgtgccttggtttcttcttctgcaaaatggagcataatgctagtgcctactttgcagagatgatgtgaggattaaaaaaggcattccatgtaaagtacttagaatagttgttggtatctggtaaacattcagcagatgttagctacttattattttcagaatttcctccatagaacatcacatacgtctaagaaaagtgagaaatttgaacccaggcattaaaaaaaaaataagattcatgtccattagcccagagataactatagtaaaaaatgtagtgcgttttattttcagacagggtctcactctgttaactcaggctggagtgcagtggcatgatcacggctcactgcaacctctgcctcctgggatcgagtgatcctcccaccttagccttccagtagctgggactacaggcacatgccaccatgcctggcttttttttttttttaatttaatattttgtggagacagggtctcaccaatattgcccaggctggtaactcctgggcccaagcagtccacccacctcggcctcccaaagtggcgggattacaggcgcgtgagacactgcacccagcctgtggtgcattttaaaatttttattagactctttggtacttagtttggttcatacaatgcttttcctcaacatttttgttctcaaaatttttaattttataataaacacatctatacctactgcttagaatctaccattagcattttactattcttgctttattacatatctattcatccatttattccctttattcatctgccaatccatcttattggacacattttgaaataaattgcagacatcagtacacttctccttaaatacttaagattggctgtcattaactggagttcagtacacatatatttttttgaggtagggtctccctctgtttcccaggctggagtacagtgatacgatctccgctcactgcaacttccgcctcccgggctcaagcagtcttcccctcaaactcctgaatagctggaaatacaagcatgagccaccacacctagctaatttttgtatttttagtagaggcagggttttggcaggttgcccaggctggtctcaaactcctgacctcaagtgatccacctgccttggcctcctgaagtgttgggattacaggcgtgagccactgcgcctggccatttttttcttttgaggcggaagttatatacagtgaaatacacaaatattcagtgagttttgagaaataacccttttttttttcgttttgaaacggagtcttgctctgtcgcccaagttggagtgcagtggtgtgatcttggctcactgcagcctctgcctcctgggttcaagcgattctcctgcctcagcctcttgagtagctgggactacaggcacgtaccaccacacccggctaatttttcatatttttagtagagatggggtttcaccgtgttagccaggatggtcttgatctcctgaccttgtgatccacctgcctcggcctcccaaagtgctgggattacaggtgtgagccacagtgcccaaccgagaaatacctttgtaacccaaattcctatgaagacaaaaaacattatcactccagagagtttcttcatgctccttatcagtcagtctttttcaccaacccccagagacaaccactgttctggagggtttttttcccttccataaattagttttgcttttctagaattttatccaaatggaactatgtcaatatgcactctaaaggcttctttcatcaagtttatttcttttgctgagtagtatttcactgtatgaatgcactacagtttattttcctattggcatagaattttaaaaaaatagagtgcacaggtctttctctttggagggtagcattaatatggacatggttgtattgagaccaacagatatgcacaaccattttactgcttctgtcacaacaaacattaaaacaaagttgggcgtggtggcgcatgcctataatcccggcgctttaagaggctgagggaggaggattgcttgagcccaggagttcgagaccagcctggacaacatggcgagaccccatctctacaaaaaatacaaaaatagctgagtgtggtgatgcgtgcctgtagtctcagcttcccgggaggctggggcaggaggattgcttgagctaaggaggttcagtctgcagtgagccatcatcgttccattgcactccagcctgggcaacagagtgagactctatttcaaaaattaaaagaaaataaataaaaattattgcttttaaaaatgcacagaaatgttctggcttctggttctaaagtatttatcccttcatctaagaataatctcatgaaaaatatattcaaaagggaagtagaaatcttttcccctgtgtttcctgaagaatcaattacttcttttttatttttctgaacagaaccatactcttggtgatattagcaatttttaaaaatctgaactcagttcaggtacaagtggaaaattacgagagtagacttattgtatttcttttccccttgagtggttttctgtttcatcaaaatgaccataaatcgaatacttcatcttatgtttatgctttaacttttgtaaatgatgcttcaatgaatatctttgcatggtcctgtatgtttttgcatctgattatttctttttaatagattttaaagtgttactgttaggccagagggttatgaacatttaaggttaatacatacttgctaaattgcttaccaaaaaggtaagcaacagtacataggaatagtcatgtgccgttgtcaattcttgttattttaaaggtagaaacaagacgaaatggagaacgcgtggtatcccttggatatacttgtttaggaaagaactaagttttttcagagtatatggagttgagctcatttcagtgttagcattatgcaagatgtgacccttccctttttagcacgaagccaatcaagaaatgagatagcgtagagtttggtcatgcattttttgccacaaatgcgactaggtatttatagggtgtgtttctgagtttctgctctgatttgtaaactgggttcttgttttttattacccagcctgaagaaagttcgtggcaaagtctggaagcaaagaatatctagccctctgttcaacaccaaacaatacacaatggaactagagcggctctatctacagatgtgggagcattatgcagctggcaacaaacctgaccacatgattaagcctgttgaagtcactgagtcagcataaataaagactgcacaggagaattacccctatacctgagcctcaaccttctgggggaaagggaactagataacatacttcttacttgtctgtacagtaccttgttgcagatgggtgatatataatggtaatagaatagcacagccagacttgcttcctgcatggtagggagagacacaaaagatgggaaactgcttttccacaaggaatctccgtagaattttgcggcgaccagatggtgcataggtctggaaggtctgatctcccttggtcttccatgggatggttagtgtggaggggagatatagattgtccggccgctttgtgattccatggattgattcagtcttctggatttttttttctttatattttgggtactggagcttttaaaaatgtttggtttcaggtatttttattcatgtgaagtgtatatgattctcttgagataaggttttaagctaaaatgttactccctgttttagtttctgaactctgacagattgacagggactttgctggtgtagtctttttataggttttataaaccacttgagcctatatcagtcgttttagtgtctgacctaatatttggagctatcagtgctttgttgatttagatgatgactcaagattttttctggtccatttcccatttccttttcttccctgacccccataccctcacccttaaaattctcctgtaactcaactaacaaaatcaagcctgattcaaaacatcctagggtgttttaaacacaccatctggtgccaaatgaagatttttaggagtgattactaattatcaagggcacagttgtggtactgtcattgataataatatagtttttttttttttcctaattttgacctgtttcaccagtgttttacccttgactgccccttctatgctgcttccaaaagtgatagtgtgtgtaagatttttaccttcctttctaaagttttttttttttttttttaagtgagtcctgttcttcctatttctttcagcagaaatgaaatcccaggtaagtataagtattcaagtatttgatcagtaagtcacagttatctccagtgcattaaataaccttcatcaagaaataggttataggtaaaatctctgaaggatcatctatgtattcaagtaattattttttagataataactgtcttctggacttggtcttgaagtctgtacagattcagcctcagtagtagcgaactgcactgctgtttggtttggagtacaaattagacttatagtcctcctggaacttgagttattaaaatcataggaataaaattatgggatctcaacaaagggtcgagggtttgaggcttaaacaagccaacatatgaatatatgttttgtctcgctatactgcacttacgctatccagttgcaggtaattttttgtctgctagtagtgttctagattatgtctttccaaagcgctgaggctgtgcacctattctgtagttgcagctgatgcctgaatgtatcctagctgacaaattattgattaataagaacttgaatttctggaagattcttactgttaaccaaattttgagcaaggagtctcaaaggtaattctgaaccagaattacatgttaatgaacagtgtaccttttaacagtgtaaatcacggaatatccgtgaagggatttcttaatttattttttaccggttgattgaaatatcagttaaaggttgccagcatggttgcagataaactgatgtttgaaattcgctgaaatacttaatgtggaataggataatatacttccaatgccctcaaggctgtgaccttacagccattttacatagcacatcattcctcctatagggatgaactttttcctggcacgaaaagtagccgctctggttgaagctttgcttattgtaacaggcttttatttccaggtaatatgtcttggaagacttaattctgattagagatatagatattactggaaactaattgttttttttctattgtactctgctttatcaaagaagtaaaacatttaaatcgtactacagaaattaagatgttgtcttgcgatccttaataaatgaatgatttccctttaatacggctgatgtattttgtatcctgttatattgagtgttggactgaagaatagtgtcatccaatgatgaaacctatatttaaacacctccaaacagcatgaaaggaccaaatttccctactgtagctatgatattaggaattgttttctcagccaggtctggtagagggtggggttagtttatgatccattaagaaggcaaagtggccttgtgtggtggctcacgcctgtaaccccagcactttgggaggcggaggcaggtggatcacttgaggtcaggagttcaagaccagcctggctaacatggtgaaaccctgtctctactaaaaatacaaaaaaactagccgggagtggtggcacacgcttgtaatcccaactacatgcgaggctgagacaggagaatcgcttgaacccgggaggcagaggttgcagtgagctgagatcgtgccattgcattccagcctggacaacagagtgagatgtcatctcaaaaaaaaaaaaagacaaagcctgtgatccccaacacacccattcatgcttgctcctcttttgcttgtgacctctctgctttcatttacccttgtagtctcggttttcatgtggaccattcttttcttggctcttgggggcaaaattattttccttcctgctctgtatagtggtcgaactctgccacaagcatcagggaggaagatatcaattcatgattgatatcatgaattgatatcatggcaaatgctgtttgccagttttctcagatatgagagggaagatttttttcagtaaaattttgcatgatttaattcccaacttcttaaagccctcatgtggattggtgtttgtttcctaaaagattattatgggaagaaagcctactcaaaagtttttactcccattcgcttctcttgaaaaatctacattatttccagtctacttcagctcctcctgccttcaatctaaaggagttgcttgcacaccttgagtaaatgtgtgtaatttctgaactgcccattttaagatttgattccaaacctatcccattcttttacttgaagcatatgcctctcaattatagctaacatcctggttctttgcccaagcctctgaggcacagttatgtaatccccattcttataaagaggcttgtagaggttaatttgcaacttcattgtagagagagagagtaatcagccccctttatccaatttttttatcaactgagaaagtatctgtatttaggtaggcaatctggtgcttaagagcaggttttggaactagagttggagtttgaatctcagctctgccacttttttttttttttttgagatggagtctcgccctgtccccatgctggagtgcaatggcacgatctcggctcactgcaacctccgcctcccaggttccagcgattctcctgcctcagcctcccgagtagctgggattacaggcgcgggccaccacacgcggctatttttttttttttatctttagtagagacggggtttcaccatgttgaccaggctggtctcaagctcctgacttcgtgatccacccgcctcagcctcccaaagtgctggggttacaggcgtgagccactgtgcccggccagctctgccactttctagctgtgtgatctctaaaatgagagtgctaacagttgcctactttttaaagtgttgtatgaattaagtcgtaagacacttggaataatgcttagcacaaaatagtatcaaacgcagggacattttatgtgtgttctgttacccaaaggcatacattacgaaatatatagatgctcgctggagaaactggcacaatctagaaagcgctgttttccccgtttgtttttcttggtgtgtatggagtgagtgccaaaaagcctcaagtaacctttttttatctccagggtttggagggttctggaagtaaacatcatagttcgccgttgccaggcgctgttgagttgctgccttgctcttatctaggggtctagatggccttccctg

SEQ ID NO: 174 - DNA, 21 bp, Artificial Sequence
  shOGA
ccagaaactttccttgctaat

SEQ ID NO: 175 - RNA, 19 nt, Artificial Sequence
  Control siRNA, sense strand
gcaguuauaaugacuagau

(19) United States
(12) Patent Application Publication
(10) Pub. No.: US 2017/0166897 A1
(43) Pub. Date: Jun. 15, 2017

(54) COMPOSITIONS AND METHODS FOR TARGETING O-LINKED N-ACETYLGLUCOSAMINE TRANSFERASE AND PROMOTING WOUND HEALING

(71) Applicant: UNIVERSITY OF NORTH CAROLINA AT CHAPEL HILL, Chapel Hill, NC (US)

(72) Inventors: David Rubenstein, Chapel Hill, NC (US); Kasper Runager, Chapel Hill, NC (US)

(73) Assignee: University of North Carolina at Chapel Hill, Chapel Hill, NC (US)

(21) Appl. No.: 14/774,800

(22) PCT Filed: Mar. 11, 2014
(86) PCT No.: PCT/US2014/023525

§ 371 Date: Sep. 11, 2015

Related U.S. Application Data
(60) Provisional application No. 61/775,937, filed on Mar. 11, 2013.

(51) Int. Cl. C12N 15/113; A61K 45/06; A61K 31/7088

(52) U.S. Cl. CPC C12N 15/1137; C12Y 204/01255; A61K 31/7088; A61K 45/06; C12N 2310/531; C12N 2310/14; C12N 2310/11

(57) ABSTRACT

The presently disclosed subject matter provides compounds, compositions, and methods for targeting UDP-N-acetylglucosamine polypeptide β-N-acetylglucosaminyl transferase (OGT). Further, the presently disclosed subject matter provides compounds, compositions, and methods for promoting wound healing.

### RELATED APPLICATION

This application claims priority from United States Provisional Patent Application Ser. No. 61/775,937, which was filed on Mar. 11, 2013, the entire disclosure of which is incorporated herein by this reference.

### GOVERNMENT INTEREST

The presently disclosed subject matter was made with support from the United States government under Grant RO1 AI49427 awarded by the National Institutes of Health. The United States government has certain rights in the invention(s) described hereinbelow.

### TECHNICAL FIELD

The presently-disclosed subject matter relates to compounds, compositions, and methods for targeting UDP-N-acetylglucosamine polypeptide β-N-acetylglucosaminyl transferase (OGT). The presently-disclosed subject matter further relates to compounds, compositions, and methods for promoting wound healing.

### BACKGROUND ART

Chronic wounds are a significant source of morbidity affecting 6.5 million patients in the United States and costing approximately $25 billion annually to treat (1). Patients with diabetes are at increased risk for developing chronic non-healing wounds. A variety of factors likely contribute to the predisposition of diabetic patients to develop chronic wounds including neuropathy, vasculopathy, as well as the underlying endocrine dysfunction that results in elevated glucose levels.

Like phosphorylation, intracellular protein O-glycosylation is a common, dynamic post-translational modification that regulates many intracellular proteins including enzymes, transcription factors, structural and cell adhesion proteins. N-acetylglucosamine (GlcNAc) modification of serine and threonine is catalyzed by the enzyme UDP-N-acetylglucosamine-polypeptide β-N-acetylglucosaminyl transferase (O-GlcNAc transferase, OGT); whereas, GlcNAc is removed by O-GlcNAc-selective N-acetyl-β-D-glucosaminidase (GlcNAcase, OGA) (reviewed in (2)).

Hyperglycemia, excess glucose, feeds into the glucosamine pathway to provide excess UDP-GlcNAc for OGT to modify intracellular proteins (3). Excess glucose is converted to glucosamine, which is ultimately converted to UDP-N-acetylglucosamine (UDP-GlcNAc), the donor substrate for OGT modification of intracellular proteins. Consequently, hyperglycemia is associated with increased O-glycosylation of a variety of proteins (3-7). The increased GlcNAc modification of intracellular proteins observed in hyperglycemic states including diabetes is thought to contribute to some of the pathology associated with diabetes. For example, (i) pancreatic-cells have high levels of OGT and are sensitive to alterations in intracellular O-GlcNAc modification and (ii) over-expression of OGT in muscle and adipose tissue causes diabetes in transgenic mouse models (8). Increased GlcNAc modification of intracellular proteins is observed in diabetic tissue, including human diabetic tissue (9) and hyperglycemic animal models (4). Further support for a pathologic role for intracellular O-glycosylation in diabetes comes from studies demonstrating a genetic association of diabetes and mutations causing increased intracellular protein O-glycosylation; a mutation that results in early termination in the gene encoding OGA has been associated with a genetic predisposition to adult onset type II diabetes in a Mexican American population (10). OGA removes GlcNAc from intracellular proteins and the identified OGA mutations result in increased levels of protein O-glycosylation.

During wound healing, keratinocytes at the wound margin must down-regulate adhesion to adjacent cells at the trailing margin to permit movement away from the edge and into the wound (11). Previous data from the group demonstrated that increased O-glycosylation stabilizes cell-cell adhesion in part by increasing the post-translational stability of desmosome components including plakoglobin (12).

### BRIEF SUMMARY

Briefly, the present disclosure is directed to compounds, compositions and methods for targeting OGT and/or for promoting wound healing.

In certain embodiments of the present disclosure, an isolated antisense OGT polynucleotide is provided, wherein the polynucleotide is 18-30 nucleotides in length and further wherein the isolated antisense OGT polynucleotide comprises a sequence that hybridizes to OGT mRNA. In some embodiments, the isolated polynucleotide may have a sequence as set forth in SEQ ID NO: 3 or a sequence complementary to an 18-30 nucleotide sequence set forth in SEQ ID NO: 1 or SEQ ID NO: 173. In some embodiments, the isolated polynucleotide hybridizes to OGT mRNA under conditions of medium to high stringency.

In some embodiments, the present disclosure is directed to a pharmaceutical composition comprising (i) an antisense OGT polynucleotide, wherein the polynucleotide is 18-30 nucleotides in length and has a sequence that hybridizes to OGT mRNA; and (ii) a pharmaceutically acceptable carrier. In some embodiments, the antisense OGT polynucleotide is a first wound healing agent and/or a first anti-OGT agent. In some embodiments, the pharmaceutical composition further comprises a second wound healing agent and/or a second anti-OGT agent.

In some embodiments, the polynucleotide of the pharmaceutical composition hybridizes to OGT mRNA under conditions of medium to high stringency. In some embodiments, the pharmaceutically-acceptable carrier is a gel, an alginate, a hydrogel, or a cellulose-based carrier selected from hydroxymethyl cellulose, hydroxyethyl cellulose, carboxymethyl cellulose, hydroxypropylmethyl cellulose, and mixtures thereof. And in certain embodiments, the pharmaceutically-acceptable carrier comprises an alcohol, a polyoxyethylene-polyoxypropylene copolymer, Pluronic® F-127 and/or mixtures thereof.

In some embodiments, the isolated polynucleotide and/or the polynucleotide of the pharmaceutical composition has a sequence complementary to an 18-30 nucleotide sequence set forth in the coding region of SEQ ID NO: 1 or SEQ ID NO: 173. In some embodiments, the isolated polynucleotide and/or the polynucleotide of the pharmaceutical composition has a sequence complementary to an 18-30 nucleotide sequence set forth in the regulatory region of SEQ ID NO: 173. And in certain embodiments, the isolated polynucleotide and/or the polynucleotide of the pharmaceutical composition has a sequence complementary to an 18-30 nucleotide sequence set forth in the intronic region of SEQ ID NO: 173.

In some embodiments, the isolated polynucleotide and/or the polynucleotide of the pharmaceutical composition comprises a sequence selected from any one of SEQ ID NOs: 4-168.

In some embodiments, the isolated polynucleotide and/or the polynucleotide of the pharmaceutical composition comprises a sequence complementary to an 18-30 nucleotide sequence set forth in SEQ ID NO: 1 or SEQ ID NO: 173 or a sequence corresponding to an 18-30 contiguous nucleotide fragment of SEQ ID NO: 3, wherein the sequence comprises a sequence selected from any one of SEQ ID NOs: 4-168. And in some embodiments, the isolated polynucleotide and/or the polynucleotide of the pharmaceutical composition comprises a fragment of any one of SEQ ID NOs: 4-168 and further comprises 1-10 nucleotide residues, such that the sequence of the polynucleotide corresponds to an 18-30 contiguous nucleotide fragment of SEQ ID NO: 3, or an 18-30 nucleotide molecule that is complementary to an 18-30 nucleotide sequence set forth in SEQ ID NO: 1 or SEQ ID NO: 173.

In some embodiments, the pharmaceutical composition is formulated for topical application and/or local injection. In certain embodiments, the pharmaceutical composition is formulated in a wound dressing. And in some embodiments the pharmaceutical composition is formulated in a colloidal gel dressing. In some embodiments, the pharmaceutical composition is formulated for sustained release, for slow release, for extended release, and/or for controlled release. And in some embodiments, the pharmaceutical composition is a liquid, a cream, an ointment, an emulsion, a lotion, a spray, a salve, a foam, and/or a paint.

In some embodiments, the isolated polynucleotide and/or the polynucleotide of the pharmaceutical composition is administered to a subject having a wound. In some embodiments, said wound is not healing at an expected rate. In certain embodiments, the subject's wound is delayed, difficult to heal and/or chronic, and in still further embodiments, the wound is characterized at least in part by increased expression of OGT.

In some embodiments, the subject is a diabetic. In certain embodiments, the subject has (i) type 1 diabetes mellitus, (ii) type 2 diabetes mellitus, (iii) higher than normal blood glucose levels and/or (iv) insulin-resistant receptors. In some embodiments, the subject is not diabetic.

In some embodiments of the presently-disclosed subject matter, a method of inhibiting OGT in a cell is provided and involves administering an anti-OGT agent to a cell, wherein the anti-OGT agent is chosen from (i) an antisense OGT polynucleotide, wherein the polynucleotide is 18-30 nucleotides in length and has a sequence that hybridizes to OGT mRNA; (ii) a double stranded RNA molecule that inhibits expression of OGT; and (iii) a short hairpin RNA molecule that inhibits expression of OGT. In some embodiments, the cell includes insulin-resistant receptors, and in certain embodiments, the cell is in a subject.

In certain embodiments of the presently-disclosed subject matter, a method of treating a subject having a wound is provided and involves administering an anti-OGT agent to the subject, wherein the anti-OGT agent is selected from the group consisting of (i) an antisense OGT polynucleotide, wherein the polynucleotide is 18-30 nucleotides in length and has a sequence that hybridizes to OGT mRNA; (ii) a double stranded RNA molecule that inhibits expression of OGT; and (iii) a short hairpin RNA molecule that inhibits expression of OGT.

In some embodiments of the methods of the present disclosure, the polynucleotide hybridizes to OGT mRNA under conditions of medium to high stringency. In some embodiments of the methods of the present disclosure, the polynucleotide has a sequence set forth in SEQ ID NO: 3, in certain embodiments, the sequence of said 18-30 nucleotides corresponds to an 18-30 contiguous nucleotide fragment of SEQ ID NO: 3, and in certain embodiments, the antisense OGT polynucleotide comprises a sequence selected from any one of SEQ ID NOs: 4-168.

In certain embodiments of the methods of the present disclosure, the polynucleotide has a sequence complementary to an 18-30 nucleotide sequence set forth in SEQ ID NO: 1 or SEQ ID NO: 173.

In some embodiments of the disclosed methods, the polynucleotide is an antisense OGT polynucleotide, and in certain embodiments, the polynucleotide is an oligodeoxynucleotide.

In some embodiments of the methods of the present disclosure, the polynucleotide comprises a sequence (i) complementary to an 18-30 nucleotide sequence set forth in the regulatory region of SEQ ID NO: 173, (ii) complementary to an 18-30 nucleotide sequence set forth in the intronic region of SEQ ID NO: 173, and/or a sequence (iii) complementary to an 18-30 nucleotide sequence set forth in SEQ ID NO: 1 or SEQ ID NO: 173 or a sequence corresponding to an 18-30 contiguous nucleotide fragment of SEQ ID NO: 3, wherein the sequence comprises a sequence selected from any one of SEQ ID NOs: 4-168.

In certain embodiments of the disclosed methods, the sequence of the polynucleotide comprises a fragment of any one of SEQ ID NOs: 4-168 and further comprises 1-10 nucleotide residues, such that the sequence of the polynucleotide corresponds to an 18-30 contiguous nucleotide fragment of SEQ ID NO: 3, or an 18-30 nucleotide molecule that is complementary to an 18-30 nucleotide sequence set forth in SEQ ID NO: 1 or SEQ ID NO: 173.

In some embodiments of the disclosed methods, the isolated, double-stranded RNA molecule includes a first strand comprising a sequence selected from SEQ ID NOS: 169-171, and including about 11 to 27 nucleotides. In certain embodiments of the methods, the small hairpin RNA comprises the sequence of SEQ ID NO: 172.

In some embodiments of the presently-disclosed subject matter, the anti-OGT agent is provided in a pharmaceutical composition. And in some embodiments, the pharmaceutical composition further comprises a pharmaceutically-acceptable carrier, wherein the pharmaceutically-acceptable carrier comprises, for example, a gel, an alcohol, a polyoxyethylene-polyoxypropylene copolymer, Pluronic® F-127, an alginate, a hydrogel, and/or a cellulose-based carrier chosen from hydroxymethyl cellulose, hydroxyethyl cellulose, carboxymethyl cellulose, hydroxypropylmethyl cellulose, and mixtures thereof.

In some embodiments of the methods of the present disclosure, the pharmaceutical composition is formulated for and/or administered by topical application, local injection, wound dressing, and/or colloidal gel dressing. In certain embodiments of the disclosed methods, the pharmaceutical composition is formulated for sustained release, slow release, extended release, or controlled release, and, further, the composition is in the form of a liquid, cream, ointment, emulsion, lotion, spray, salve, foam or paint.

In some embodiments of the disclosed methods, the pharmaceutical composition is administered topically and/or by local injection. In certain embodiments, the pharmaceutical composition or agent is administered to treat a subject having a wound, and in some embodiments, the wound is delayed, difficult to heal, not healing at an expected rate and/or chronic. In certain embodiments, the wound is characterized at least in part by increased expression of OGT.

In some embodiments of the methods of the present disclosure, (i) the subject is a diabetic, (ii) the subject has type 1 diabetes mellitus, (iii) the subject has type 2 diabetes mellitus, (iv) the subject has higher than normal blood glucose levels, (v) the subject has insulin-resistant receptors, and/or (vi) the subject is not diabetic.

In some embodiments, the disclosed methods further comprise the step of administering a second wound healing agent and/or a second anti-OGT agent to a cell or to a subject.

It is to be understood that both the foregoing general description and the following detailed description present embodiments of the disclosure and are intended to provide an overview or framework for understanding the nature and character of the disclosure as it is claimed. The description serves to explain the principles and operations of the claimed subject matter and sets forth illustrative embodiments, in which the principles of the invention(s) are used. Other and further features and advantages of the present disclosure will be readily apparent to those skilled in the art upon reading the following disclosure and considering the accompanying drawings.

### DESCRIPTION OF THE FIGURES

FIG. 1. Hyperglycemia increases O-GlcNAcylation and retards wound healing in human keratinocytes. HaCaT cells were cultured in media supplemented with glucose to the final concentrations indicated. Cell lysates were analyzed by SDS-PAGE and immunoblotting using RL2 antibody, which recognizes O-GlcNAc modifications and GAPDH as a loading control (FIG. 1). The RL2 signal was quantified relative to the GAPDH loading control (FIG. 1B). Monolayers of human keratinocytes were incubated in DMEM with the glucose concentrations indicated. Cells were scratched with a pipet tip and micrographs were made at 0 h and 16 h (FIG. 1C). Wound sizes were then measured using image analysis software. N=11 for all conditions (FIG. 1D). Error bars reflect the standard error of mean (SEM). * indicates P-value<0.07 and ** indicates P-value<0.0005 compared to normal glucose levels (5.5 mM). The P-value between 25 and 100 mM is <0.01.

FIG. 2. RNA interference (RNAi) in the O-GlcNAc pathway affects wound healing rates in human keratinocytes. Moreover, OGT knockdown using shRNA accelerates wound healing. HaCaT cells were stably transduced with shRNA targeting OGT, OGA, and GFP (control) and grown to confluency. Cell lysates were then analyzed by immunoblotting probing against O-GlcNAc modifications (RL2), OGT protein, and actin (loading control) (FIG. 2A). RL2 reactivity was quantified compared to actin signal (FIG. 2B). Transduced cells were grown to confluency in growth medium with 25 mM glucose and scratched to introduce wounds. Representative micrographs obtained at 0 h and 12 h are shown in FIG. 2C, while the quantification of open wound areas is shown in FIG. 2D. In the scratch wounding assay N=18 for untreated cells, N=10 for shGFP, N=8 for shOGT, and N=14 for shOGA. Error bars reflect the standard error of mean (SEM). Asterisks (*) denote results with p-values<0.05.

FIG. 3. Specific knock down of OGT accelerates human keratinocyte wound healing. HaCaT cells were transfected with 100 nM siRNA against OGT or a scrambled control siRNA. Cell lysates of confluent cultures were immunoblotted probing for RL2, anti-OGT, and anti-GAPDH reactivity (FIG. 3A) and quantified (FIG. 3B). 60 hrs after transfection with siRNAs the confluent cells were scratched and micrographs were obtained at 0 h, 16 h, and 26 h (FIG. 3C). The open wound area was quantified using image analysis software (FIG. 3D). N=7 for the untransfected cells, N=7 for control siRNA, and N=6 for OGT siRNA. Error bars reflect the standard error. The asterisk indicates p value <0.05 as compared with controls.

FIG. 4. Cell-cell adhesion is increased in OGT transfected keratinocytes. FIG. 4A presents the results of a dispase assay, wherein confluent monolayer cultures of control (Con) and OGT (OGT) transfected keratinocytes were floated off the plates after treatment with dispase and subjected to shear force by rocking the cultures back and forth 10 times. Fewer fragments are generated from the OGT cells indicating greater cell-cell adhesion compared to controls. FIG. 4B and FIG. 4C present electron micrographs of control and OGT over-expressing keratinocytes, wherein FIG. 4B provides a top down view and FIG. 4C. provides an orthogonal view, each showing that the cell membranes of OGT over-expressing keratinocytes are more tightly associated compared to control keratinocytes.

FIG. 5. Increased O-glycosylation of intracellular proteins is observed in skin of diabetic mice compared to control mice. FIG. 5 presents an immunoblot analysis using the GlcNAc-specific monoclonal antibody RL2, which demonstrates increased GlcNAc modification in epidermal extracts of diabetic mice compared to wild type controls. The asterisks denote additional GlcNAc modification of proteins detected in diabetic skin not seen in controls. Arrows depict GlcNAc modification of proteins not altered in diabetic skin vs. controls and serve as loading controls (+, anode; −, cathode).

FIG. 6. Increased intracellular O-glycosylation delays wound healing. Confluent monolayer cultures of control (Con) and OGT (OGT) transfected keratinocytes were subjected to wounding by scratch and the time to closure of the wound determined. As shown in FIG. 6, control keratinocytes heal the wound by 16 hours; whereas, OGT over-expressing keratinocytes have failed to close the wound at t=16 hours.

FIG. 7. Inhibition of OGT activity using OGT specific shRNA promotes keratinocyte wound healing and reverses the dose dependent inhibition of wound healing by glucose in a monolayer scratch assay. Confluent cultures of human HaCat Control (darker colored bars) and HaCat keratinocytes in which OGT was knocked down using an OGT specific shRNA (shOGT, lighter colored bars) were treated with increasing concentrations of glucose, subjected to wounding by scratch, and the size of the healing wound measured 19 hrs post wounding (n=12 per group), as presented in FIG. 7A. Scratch assay as in FIG. 7A for non-transduced control HaCat keratinocytes and GFP specific shRNA (shGFP) transduced controls compared to shOGT transduced HaCat keratinocytes (n=12 per group), data is presented in FIG. 7B.

FIG. 8. shRNA knockdown of OGT decreases keratinocyte intracellular protein O-glycosylation; whereas, OGA knockdown increases protein O-glycosylation. HaCaT cells stably transfected with shRNA targeting GFP (control), OGT, and OGA were grown to confluency. Cell lysates were then analyzed by immunoblotting with antibodies to the (i) O-GlcNAc modification (RL2), (ii) OGT protein, and (iii) GAPDH (loading control), as shown in FIG. 8A and FIG. 8B. As shown, genetic knockdown of OGT decreases O-GlcNAc protein modification; whereas, genetic knockdown of OGA increases O-GlcNAc protein modification.

FIG. 9. OGT antisense oligodeoxynucleotides downregulate OGT protein levels and O-GlcNAC modification in skin of test mice when applied topically to a wound. Topical application of 10 nM OGT antisense oligodeoxynucleotides in 50 ul Pluronic® F-127 gel to full thickness skin wound of WT mice at t=0 and t=24 hrs post wounding. Perilesional skin of mice treated with OGT antisense ODN or vehicle control was harvested at 48 hrs post wounding. Skin extracts were separated by SDSPAGE and probed by immunoblot with antibodies to the O-GlcNAc modification (RL2), OGT protein, or GAPDH (as a loading control), as shown in FIG. 9.

### DETAILED DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

Each example is provided by way of explanation of the present disclosure and is not a limitation thereon. In fact, it will be apparent to those skilled in the art that various modifications and variations can be made to the teachings of the present disclosure without departing from the scope of the disclosure. For instance, features illustrated or described as part of one embodiment can be used with another embodiment to yield a still further embodiment.

All references to singular characteristics or limitations of the present disclosure shall include the corresponding plural characteristic(s) or limitation(s) and vice versa, unless otherwise specified or clearly implied to the contrary by the context in which the reference is made.

All combinations of method or process steps as used herein can be performed in any order, unless otherwise specified or clearly implied to the contrary by the context in which the referenced combination is made.

The methods and compositions of the present disclosure, including components thereof, can comprise, consist of, or consist essentially of the essential elements and limitations of the embodiments described herein, as well as any additional or optional components or limitations described herein or otherwise useful.

The presently-disclosed subject matter includes compounds, compositions, and methods that are useful for inhibiting UDP-N-acetylglucosamine polypeptide β-N-acetylglucosaminyl transferase (OGT) in a cell and/or for wound healing.

In some embodiments, the presently-disclosed subject matter includes an isolated antisense OGT polynucleotide having a sequence that hybridizes to OGT mRNA.

The term “isolated”, when used in the context of an isolated polynucleotide, is a polynucleotide that, by the hand of man, exists apart from its native environment and is therefore not a product of nature.

The terms “nucleotide”, “polynucleotide”, “nucleic acid” and “nucleic acid sequence” refer to deoxyribonucleotide(s) or ribonucleotide(s) and polymer(s) thereof in either single or double stranded form.

Synthesis of antisense polynucleotides and other anti-OGT polynucleotides such as siRNA and shRNAs is known to those of skill in the art. See e.g. Stein C. A. and Krieg A. M. (eds), Applied Antisense Oligonucleotide Technology, 1998 (Wiley-Liss). The antisense polynucleotide can inhibit transcription and/or translation of an OGT. In some embodiments, the polynucleotide is a specific inhibitor of transcription and/or translation from the OGT gene or mRNA, and does not inhibit transcription and/or translation from other genes or mRNAs. The product may bind to the OGT gene or mRNA either (i) 5′ to the coding sequence, and/or (ii) to the coding sequence, and/or (iii) 3′ to the coding sequence.

The antisense polynucleotide is generally antisense to an OGT mRNA (e.g., complementary to a sequence set forth in SEQ ID NO: 1 or SEQ ID NO: 173). Such a polynucleotide may be capable of hybridizing to the OGT mRNA and may thus inhibit the expression of OGT by interfering with one or more aspects of OGT mRNA metabolism including transcription, mRNA processing, mRNA transport from the nucleus, translation or mRNA degradation. The antisense polynucleotide typically hybridizes to the OGT mRNA to form a duplex which can cause direct inhibition of translation and/or destabilization of the mRNA. Such a duplex may be susceptible to degradation by nucleases.

The term “complementary” refers to two nucleotide sequences that comprise antiparallel nucleotide sequences capable of pairing with one another upon formation of hydrogen bonds between the complementary base residues in the antiparallel nucleotide sequences. As is known in the art, the nucleic acid sequences of two complementary strands are the reverse complement of each other when each is viewed in the 5′ to 3′ direction. As is also known in the art, two sequences that hybridize to each other under a given set of conditions do not necessarily have to be 100% fully complementary.

The antisense polynucleotide may hybridize to all or part of the OGT mRNA. Typically the antisense polynucleotide hybridizes to the ribosome binding region or the coding region of the OGT mRNA. The polynucleotide may be complementary to all of or a region of the OGT mRNA.

For example, the polynucleotide may be the exact complement of all or a part of OGT mRNA. However, absolute complementarity is not required and polynucleotides which have sufficient complementarity to form a duplex having a melting temperature of greater than about 20° C., 30° C., or 40° C. under physiological conditions are particularly suitable for use in the present invention.

Thus the polynucleotide is typically a homologue of a sequence complementary to the mRNA. The polynucleotide may be a polynucleotide which hybridizes to the OGT mRNA under conditions of medium to high stringency such as 0.03M sodium chloride and 0.03M sodium citrate at from about 50° C. to about 60° C. In some embodiments, the phrase “medium to high stringency” means between about 0.0165 and about 0.033 M sodium chloride; in some embodiments, “medium to high stringency” means between about 0.0165 and about 0.033 M sodium citrate; in some embodiments, “medium to high stringency” means a temperature of from about 5 to about 30° C. below a melting temperature (Tm), wherein 50% hybridization occurs at Tm.

In some embodiments, suitable polynucleotides are from about 6 to 40 nucleotides in length. In some embodiments, suitable polynucleotides are from about 18 to 30 nucleotides in length. In some embodiments, the polynucleotides can be about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides in length. In some embodiments, the polynucleotide is an oligodeoxynucleotide.

In some embodiments, polynucleotide can have a sequence set forth in SEQ ID NO: 3, or a sequence complementary to a sequence set forth in SEQ ID NO: 1. In some embodiments, the polynucleotide hybridizes to OGT mRNA under conditions of medium to high stringency.

In some embodiments, the polynucleotide can have a sequence complementary to a nucleotide sequence set forth in the coding region of SEQ ID NO: 1 or SEQ ID NO: 173. In some embodiments, the polynucleotide can have a sequence complementary to a nucleotide sequence set forth in the regulatory region of SEQ ID NO: 173. In some embodiments, the polynucleotide can have a sequence complementary to a nucleotide sequence set forth in the intronic region of SEQ ID NO: 173.

In some embodiments, the polynucleotide can include a sequence selected from any one of SEQ ID NOs: 4-168. In some embodiments, the polynucleotide has a sequence selected from any one of SEQ ID NOs: 4-168.

The presently-disclosed subject matter further includes a pharmaceutical composition. In some embodiments, the pharmaceutical composition includes an anti-OGT agent and a pharmaceutically acceptable carrier. As used herein, anti-OGT agent refers to OGT inhibitors and polynucleotides, such as siRNAs, shRNAs, antisense polynucleotides, such as oligodeoxynucleotides, and all such specific polynucleotides as disclosed herein, including polynucleotides having a sequence of any one of SEQ ID NOs: 4-172.

In some embodiments, the pharmaceutical composition includes a polynucleotide having a sequence of any one of SEQ ID NOs: 4-172, and a pharmaceutically acceptable carrier.

In some embodiments, the pharmaceutical composition includes a polynucleotide having a sequence that hybridizes to OGT mRNA, and a pharmaceutically acceptable carrier. In some embodiments, the pharmaceutical composition includes a polynucleotide having a sequence set forth in SEQ ID NO: 3 or a sequence complementary to a sequence set forth in SEQ ID NO: 1 or SEQ ID NO: 173, and a pharmaceutically acceptable carrier. In some embodiments, the pharmaceutical composition includes a polynucleotide as described herein, and a pharmaceutically acceptable carrier.

As used herein, the term “pharmaceutically acceptable carrier” refers to sterile aqueous or non-aqueous solutions, dispersions, suspensions or emulsions, as well as sterile powders for reconstitution into sterile injectable solutions or dispersions just prior to use. Examples of suitable aqueous and non-aqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials, such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

In some embodiments, the pharmaceutically-acceptable carrier comprises a gel, an alginate, a hydrogel, an alcohol, and/or a polyoxyethylene-polyoxypropylene copolymer. In some embodiments, the pharmaceutically-acceptable carrier comprises Pluronic® F-127. In some embodiments, the pharmaceutically-acceptable carrier comprises a cellulose-based carrier, e.g., hydroxymethyl cellulose, hydroxyethyl cellulose, carboxymethyl cellulose, hydroxypropylmethyl cellulose, and mixtures thereof. In some embodiments, the composition is a liquid, cream, ointment, emulsion, lotion, spray, salve, foam, or paint.

Compositions of the presently-disclosed subject matter can be formulated for various desirable forms of administration. In some embodiments, the composition is formulated for topical application. In some embodiments, the composition is formulated for local injection. In some embodiments, the composition is formulated in a wound dressing. In some embodiments, the composition is formulated in a colloidal gel dressing. In some embodiments, the composition is formulated for sustained release. In some embodiments, the composition is formulated for slow release, extended release, or controlled release.

The term “wound dressing” refers to a dressing for topical application to a wound and excludes compositions suitable for systemic administration. For example, the one or more anti-OGT polynucleotides, (such as OGT antisense polynucleotides) can be dispersed in or on a solid sheet of wound contacting material such as a woven or nonwoven textile material, or may be dispersed in a layer of foam such as polyurethane foam, or in a hydrogel such as a polyurethane hydrogel, a polyacrylate hydrogel, gelatin, carboxymethyl cellulose, pectin, alginate, and/or hyaluronic acid hydrogel, for example in a gel or ointment. In certain embodiments the one or more anti-OGT polynucleotides are dispersed in or on a biodegradable sheet material that provides sustained release of the active ingredients into the wound, for example a sheet of freeze-dried collagen, freeze-dried collagen/alginate mixtures (available under the Registered Trade Mark FIBRACOL from Johnson & Johnson Medical Limited) or freeze-dried collagen/oxidized regenerated cellulose (available under the Registered Trade Mark PROMOGRAN from Johnson & Johnson Medical Limited).

As used herein, “wound promoting matrix” includes for example, synthetic or naturally occurring matrices such as collagen, acellular matrix, crosslinked biological scaffold molecules, tissue based bioengineered structural framework, biomanufactured bioprostheses, and other implanted structures such as for example, vascular grafts suitable for cell infiltration and proliferation useful in the promotion of wound healing. Additional suitable biomatrix material may include chemically modified collagenous tissue to reduce antigenicity and immunogenicity. Other suitable examples include collagen sheets for wound dressings, antigen-free or antigen reduced acellular matrix (Wilson G J et al. (1990) Trans Am Soc Artif Intern 36:340-343) or other biomatrix which have been engineered to reduce the antigenic response to the xenograft material. Other matrices useful in promotion of wound healing may include for example, processed bovine pericardium proteins comprising insoluble collagen and elastin (Courtman D W et al. (1994) J Biomed Mater Res 28:655-666) and other acellular tissue which may be useful for providing a natural microenvironment for host cell migration to accelerate tissue regeneration (Malone J Metal. (1984) J Vase Surg 1:181-91). The invention contemplates a synthetic or natural matrix comprising one or more anti-OGT polypeptides described herein, including anti-OGT polypeptides. OGT antisense oligodeoxynucleotides are preferred.

Isolated polynucleotides and compositions of the presently-disclosed subject matter are useful for inhibiting OGT in a cell and/or for treating a wound in a subject. In some embodiments, the wound is not healing at an expected rate. In some embodiments, the wound is delayed, difficult to heal, or chronic. In some embodiments, the wound is characterized at least in part by increased expression of OGT or by increased activity of OGT.

As used herein, the term “wound” includes an injury to any tissue, including for example, delayed or difficult to heal wounds, and chronic wounds. Examples of wounds may include both open and closed wounds. The term “wound” may also include for example, injuries to the skin and subcutaneous tissue initiated in different ways (e.g., pressure sores from extended bed rest and wounds induced by trauma) and with varying characteristics. Wounds may be classified into one of four grades depending on the depth of the wound: i) Grade I wounds limited to the epithelium; ii) Grade II wounds extending into the dermis; iii) Grade III wounds extending into the subcutaneous tissue; and iv) Grade IV (or full-thickness wounds) wounds wherein bones are exposed (e.g., a bony pressure point such as the greater trochanter or the sacrum).

The term “partial thickness wound” refers to wounds that encompass Grades I-III. Examples of partial thickness wounds include pressure sores, venous stasis ulcers, and diabetic ulcers. The present invention contemplates treating all wounds of a type that do not heal at expected rates, including, delayed-healing wounds, incompletely healing wounds, and chronic wounds.

“Wound that does not heal at the/an expected rate” means an injury to any tissue, including delayed or difficult to heal wounds (including delayed or incompletely healing wounds), and chronic wounds. Examples of wounds that do not heal at the expected rate include ulcers, such as diabetic ulcers, diabetic foot ulcers, vascultic ulcers, arterial ulcers, venous ulcers, venous stasis ulcers, pressure ulcers, decubitus ulcers, infectious ulcers, trauma-induced ulcers, burn ulcers, ulcerations associated with pyoderma gangrenosum, and mixed ulcers. Other wounds that do not heal at expected rates include dehiscent wounds

As used herein, a delayed or difficult to heal wound may include, for example, a wound that is characterized at least in part by 1) a prolonged inflammatory phase, 2) a slow forming extracellular matrix, and/or 3) a decreased rate of epithelialization or closure.

The term “chronic wound” generally refers to a wound that has not healed. Wounds that do not heal within three months, for example, are considered chronic. Chronic wounds include venous ulcers, venous stasis ulcers, arterial ulcers, pressure ulcers, diabetic ulcers, diabetic foot ulcers, vasculitic ulcers, decubitus ulcers, burn ulcers, trauma-induced ulcers, infectious ulcers, mixed ulcers, and pyoderma gangrenosum. The chronic wound may be an arterial ulcer which comprises ulcerations resulting from complete or partial arterial blockage. The chronic wound may be a venous or venous stasis ulcer which comprises ulcerations resulting from a malfunction of the venous valve and the associated vascular disease. In certain embodiments a method of treating a chronic wound is provided where the chronic wound is characterized by one or more of the following AHCPR stages of pressure ulceration: stage 1, stage 2, stage 3, and/or stage 4.

As used herein, chronic wound may refer to, for example, a wound that is characterized at least in part by one or more of (i) a chronic self-perpetuating state of wound inflammation, (ii) a deficient and defective wound extracellular matrix, (iii) poorly responding (senescent) wound cells especially fibroblasts, limiting extracellular matrix production, and/or (iv) failure of re-epithelialization due in part to lack of the necessary extracellular matrix orchestration and lack of scaffold for migration. Chronic wounds may also be characterized by 1) prolonged inflammation and proteolytic activity leading to ulcerative lesions, including for example, diabetic, pressure (decubitus), venous, and arterial ulcers; 2) progressive deposition of matrix in the affected area, 3) longer repair times, 4) less wound contraction, 5) slower re-epithelialization, and 6) increased thickness of granulation tissue.

Exemplary chronic wounds may include “pressure ulcers.” Exemplary pressure ulcers may be classified into 4 stages based on AHCPR (Agency for Health Care Policy and Research, U.S. Department of Health and Human Services) guidelines. A stage 1 pressure ulcer is an observable pressure related alteration of intact skin whose indicators as compared to the adjacent or opposite area on the body may include changes in one or more of the following: skin temperature (warmth or coolness), tissue consistency (firm or boggy feel) and/or sensation (pain, itching). The ulcer appears as a defined area of persistent redness in lightly pigmented skin, whereas in darker skin tones, the ulcer may appear with persistent red, blue, or purple hues. Stage 1 ulceration may include nonblanchable erythema of intact skin and the heralding lesion of skin ulceration. In individuals with darker skin, discoloration of the skin, warmth, edema, induration, or hardness may also be indicators of stage 1 ulceration. Stage 2 ulceration may be characterized by partial thickness skin loss involving epidermis, dermis, or both. The ulcer is superficial and presents clinically as an abrasion, blister, or shallow crater. Stage 3 ulceration may be characterized by full thickness skin loss involving damage to or necrosis of subcutaneous tissue that may extend down to, but not through, underlying fascia. The ulcer presents clinically as a deep crater with or without undermining of adjacent tissue. Stage 4 ulceration may be characterized by full thickness skin loss with extensive destruction, tissue necrosis, or damage to muscle, bone, or supporting structures (e.g., tendon, joint capsule). In certain embodiments a method of treating a chronic wound is provided where the chronic wound is characterized by one or more of the following AHCPR stages of pressure ulceration: stage 1, stage 2, stage 3, and/or stage 4.

Exemplary chronic wounds may also include “decubitus ulcers.” Exemplary decubitus ulcers may arise as a result of prolonged and unrelieved pressure over a bony prominence that leads to ischemia. The wound tends to occur in patients who are unable to reposition themselves to off-load weight, such as paralyzed, unconscious, or severely debilitated persons. As defined by the U.S. Department of Health and Human Services, the major preventive measures include identification of high-risk patients; frequent assessment; and prophylactic measures such as scheduled repositioning, appropriate pressure-relief bedding, moisture barriers, and adequate nutritional status. Treatment options may include for example, pressure relief, surgical and enzymatic debridement, moist wound care, and control of the bacterial load. In certain embodiments a method of treating a chronic wound is provided wherein the chronic wound is characterized by decubitus ulcer or ulceration, which results from prolonged, unrelieved pressure over a bony prominence that leads to ischemia.

Chronic wounds may also include “arterial ulcers.” Chronic arterial ulcers are generally understood to be ulcerations that accompany arteriosclerotic and hypertensive cardiovascular disease. They are painful, sharply marginated, and often found on the lateral lower extremities and toes. Arterial ulcers may be characterized by complete or partial arterial blockage, which may lead to tissue necrosis and/or ulceration. Signs of arterial ulcer may include, for example, pulselessness of the extremity; painful ulceration; small, punctate ulcers that are usually well circumscribed; cool or cold skin; delayed capillary return time (briefly push on the end of the toe and release, normal color should return to the toe in about 3 seconds or less); atrophic appearing skin (for example, shiny, thin, dry); and loss of digital and pedal hair. In certain embodiments a method of treating a chronic wound is provided wherein the chronic wound is characterized by arterial ulcers or ulcerations due to complete or partial arterial blockage.

Exemplary chronic wounds may include “venous ulcers.” Exemplary venous ulcers are the most common type of ulcer affecting the lower extremities and may be characterized by malfunction of the venous valve. The normal vein has valves that prevent the backflow of blood. When these valves become incompetent, the backflow of venous blood causes venous congestion. Hemoglobin from the red blood cells escapes and leaks into the extravascular space, causing the brownish discoloration commonly noted. It has been shown that the transcutaneous oxygen pressure of the skin surrounding a venous ulcer is decreased, suggesting that there are forces obstructing the normal vascularity of the area. Lymphatic drainage and flow also plays a role in these ulcers. The venous ulcer may appear near the medial malleolus and usually occurs in combination with an edematous and indurated lower extremity; it may be shallow, not too painful and may present with a weeping discharge from the affected site. In certain embodiments a method of treating a chronic wound is provided wherein the chronic wound is characterized by venous ulcers or ulcerations due to malfunction of the venous valve and the associated vascular disease. In certain embodiments a method of treating a chronic wound is provided wherein the chronic wound is characterized by arterial ulcers or ulcerations due to complete or partial arterial blockage.

Exemplary chronic wounds may include “venous stasis ulcers.” Stasis ulcers are lesions associated with venous insufficiency are more commonly present over the medial malleolus, usually with pitting edema, varicosities, mottled pigmentation, erythema, and nonpalpable petechiae and purpura. The stasis dermatitis and ulcers are generally pruritic rather than painful. Exemplary venous stasis ulcers may be characterized by chronic passive venous congestion of the lower extremities results in local hypoxia. One possible mechanism of pathogenesis of these wounds includes the impediment of oxygen diffusion into the tissue across thick perivascular fibrin cuffs. Another mechanism is that macromolecules leaking into the perivascular tissue trap growth factors needed for the maintenance of skin integrity. Additionally, the flow of large white blood cells slows due to venous congestion, occluding capillaries, becoming activated, and damaging the vascular endothelium to predispose to ulcer formation. In certain embodiments a method of treating a chronic wound is provided wherein the chronic wound is characterized by venous ulcers or ulcerations due to malfunction of the venous valve and the associated vascular disease. In certain embodiments a method of treating a chronic wound is provided wherein the chronic wound is characterized by venous stasis ulcers or ulcerations due to chronic passive venous congestion of the lower extremities and/or the resulting local hypoxia.

Exemplary chronic wounds may include “diabetic ulcers.” Diabetic patients are prone to ulcerations, including foot ulcerations, due to both neurologic and vascular complications. Peripheral neuropathy can cause altered or complete loss of sensation in the foot and/or leg. Diabetic patients with advanced neuropathy loose all ability for sharp-dull discrimination. Any cuts or trauma to the foot may go completely unnoticed for days or weeks in a patient with neuropathy. It is not uncommon to have a patient with neuropathy notice that the ulcer “just appeared” when, in fact, the ulcer has been present for quite some time. For patients of neuropathy, strict glucose control has been shown to slow the progression of the disease. Charcot foot deformity may also occur as a result of decreased sensation. People with “normal” feeling in their feet have the ability to sense automatically when too much pressure is being placed on an area of the foot. Once identified, our bodies instinctively shift position to relieve this stress. A patient with advanced neuropathy looses this ability to sense the sustained pressure insult, as a result, tissue ischemia and necrosis may occur leading to for example, plantar ulcerations. Additionally, microfractures in the bones of the foot, if unnoticed and untreated, may result in disfigurement, chronic swelling and additional bony prominences. Microvascular disease is one of the significant complications for diabetics, which may also lead, to ulcerations. In certain embodiments a method of treating a chronic wound is provided wherein the chronic wound is characterized by diabetic foot ulcers and/or ulcerations due to both neurologic and vascular complications of diabetes.

Exemplary chronic wounds can include “traumatic ulcers.” Formation of traumatic ulcers may occur as a result of traumatic injuries to the body. These injuries include, for example, compromises to the arterial, venous or lymphatic systems; changes to the bony architecture of the skeleton; loss of tissue layers-epidermis, dermis, subcutaneous soft tissue, muscle or bone; damage to body parts or organs and loss of body parts or organs. In certain embodiments, a method of treating a chronic wound is provided wherein the chronic wound is characterized by ulcerations associated with traumatic injuries to the body.

Exemplary chronic wounds can include “burn ulcers”, including 1st degree burn (i.e. superficial, reddened area of skin); 2nd degree burn (a blistered injury site which may heal spontaneously after the blister fluid has been removed); 3rd degree burn (burn through the entire skin and usually require surgical intervention for wound healing); scalding (may occur from scalding hot water, grease or radiator fluid); thermal (may occur from flames, usually deep burns); chemical (may come from acid and alkali, usually deep burns); electrical (either low voltage around a house or high voltage at work); explosion flash (usually superficial injuries); and contact burns (usually deep and may occur from muffler tail pipes, hot irons and stoves). In certain embodiments, a method of treating a chronic wound is provided wherein the chronic wound is characterized by ulcerations associated with burn injuries to the body.

Exemplary chronic wounds can include “vasculitic ulcers.” Vasculitic ulcers also occur on the lower extremities and are painful, sharply marginated lesions, which may have associated palpable purpuras and hemorrhagic bullae. The collagen diseases, septicemias, and a variety of hematological disorders (e.g., thrombocytopenia, dysproteinemia) may be the cause of this severe, acute condition.

Exemplary chronic wounds can include pyoderma gangrenosum. Pyoderma gangrenosum occurs as single or multiple, very tender ulcers of the lower legs. A deep red to purple, undermined border surrounds the purulent central defect. Biopsy typically fails to reveal a vasculitis. In half the patients it is associated with a systemic disease such as ulcerative colitis, regional ileitis, or leukemia. In certain embodiments, a method of treating a chronic wound is provided wherein the chronic wound is characterized by ulcerations associated with pyoderma gangrenosum.

Exemplary chronic wounds can include infectious ulcers. Infectious ulcers follow direct inoculation with a variety of organisms and may be associated with significant regional adenopathy. Mycobacteria infection, anthrax, diphtheria, blastomyosis, sporotrichosis, tularemia, and cat-scratch fever are examples. The genital ulcers of primary syphilis are typically nontender with a clean, firm base. Those of chancroid and granuloma inguinale tend to be ragged, dirty, and more extravagant lesions. In certain embodiments, a method of treating a chronic wound is provided wherein the chronic wound is characterized by ulcerations associated with infection.

As used herein, the term “dehiscent wound” refers to a wound, usually a surgical wound, which has ruptured or split open. In certain embodiments, a method of treating a wound that does not heal at the expected rate is provided wherein the wound is characterized by dehiscence.

In some embodiments, composition of the presently-disclosed subject matter can further include one or more additional wound healing agent, e.g., such as those described in U.S. Pat. Nos. 8,063,023 and 8,247,384, and U.S. Patent Application Publication No. US 1012/0289479 (which are each incorporated herein by this reference), including, for example, anti-connexin agents.

In some embodiments, composition of the presently-disclosed subject matter can further include one or more additional anti-OGT agents.

As used herein, the term “subject” includes both human and animal subjects. Thus, veterinary therapeutic uses are provided in accordance with the presently disclosed subject matter. As such, the presently disclosed subject matter provides for the treatment of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses.

In some embodiments, the subject is not diabetic. In some embodiments, the subject is diabetic. In some embodiments, the subject has type 1 diabetes mellitus, type 2 diabetes mellitus, has higher than normal blood glucose levels, and/or has insulin-resistant receptors.

As noted herein, the presently-disclosed subject matter further includes a method of inhibiting OGT in a cell and a method of treating a subject having a wound.

In some embodiments, a method of inhibiting OGT in a cell involves administering an anti-OGT agent to the cell. In some embodiments, the cell is in a subject. In some embodiments, a method of treating a subject having a wound, comprising administering an anti-OGT agent to the subject.

In some embodiments of the methods of the presently-disclosed subject matter, the anti-OGT agent is selected from the group consisting of an antisense OGT polynucleotide, as describe herein, having a sequence that hybridizes to OGT mRNA; a double stranded RNA molecule that inhibits expression of OGT, e.g., siRNA, as described herein; and a short hairpin RNA molecule that inhibits expression of OGT, as described herein.

In some embodiments, the method includes administering a second wound healing agent and/or a second anti-OGT agent. In some embodiments the anti-OGT agent is provided in a composition, as described herein, for administration in accordance with the methods of the presently-disclosed subject matter.

In certain instances, nucleotides and polypeptides disclosed herein are included in publicly-available databases, such as GENBANK® and SWISSPROT. Information including sequences and other information related to such nucleotides and polypeptides included in such publicly-available databases are expressly incorporated by reference. Unless otherwise indicated or apparent the references to such publicly-available databases are references to the most recent version of the database as of the filing date of this Application.

While the terms used herein are believed to be well understood by one of ordinary skill in the art, definitions are set forth herein to facilitate explanation of the presently-disclosed subject matter.

Unless defined otherwise, all technical and scientific terms used herein have the same meaning as commonly understood by one of ordinary skill in the art to which the presently-disclosed subject matter belongs. Although any methods, devices, and materials similar or equivalent to those described herein can be used in the practice or testing of the presently-disclosed subject matter, representative methods, devices, and materials are now described.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

The presently-disclosed subject matter is further illustrated by the following specific but non-limiting examples. The following examples include certain prophetic examples, as will be apparent. The following examples may include compilations of data that are representative of data gathered at various times during the course of development and experimentation related to the present invention.

### EXAMPLES

### Example 1

This study was undertaken to explore whether increased O-GlcNAc modification of cellular proteins in diabetic skin could contribute to the delayed wound healing observed in patients with chronic diabetic skin ulcers. The present inventors modeled hyperglycemia by culturing human keratinocytes in elevated glucose. Under hyperglycemic conditions, the present inventors observed (i) increased levels of O-GlcNAc modification of keratinocyte proteins and importantly (ii) delays in wound closure. Hyperglycemia induced delays in wound closure were reversed by shRNA and siRNA knock down of OGT, the gene responsible for adding the GlcNAc moiety to proteins. These observations suggest that targeting OGT may be beneficial for treating non-healing diabetic wounds.

Non-healing wounds are a significant source of morbidity. This is particularly true for diabetic patients, who tend to develop chronic skin wounds. O-glycosylation of serine and threonine residues is a common regulatory post-translational modification analogous to protein phosphorylation; increased intracellular protein O-glycosylation has been observed in diabetic and hyperglycemic states. Two intracellular enzymes, UDP-N-acetylglucosamine-polypeptide β-N-acetylglucosaminyl transferase (OGT) and O-GlcNAc-selective N-acetyl-β-D-glucosaminidase (OGA), mediate addition and removal, respectively, of N-acetylglucosamine (GlcNAc) from intracellular protein substrates. Alterations in O-GlcNAc modification of intracellular proteins is linked to diabetes and the increased levels of protein O-glycosylation observed in diabetic tissues may in part explain some of the observed underlying pathophysiology that contributes to delayed wound healing. The present inventors have previously shown that increasing protein O-glycosylation by over-expression of OGT in murine keratinocytes results in elevated protein O-glycosylation and a hyper-adhesive phenotype. This study was undertaken to explore whether increased O-GlcNAc modification of cellular proteins in diabetic skin could contribute to the delayed wound healing observed in patients with chronic diabetic skin ulcers. In the present study, the present inventors show human keratinocytes cultured under elevated hyperglycemic conditions display increased levels of O-GlcNAc modification as well as a delay in the rate of wound closure in vitro. The present inventors further show that specific knock-down of OGT by RNA interference (RNAi) reverses this effect significantly, thereby opening up the opportunity for OGT-targeted therapeutic intervention in delayed wound healing in diabetic patients.

### EXPERIMENTAL PROCEDURES

Materials.

Cell culture media were obtained from Invitrogen, Carlsbad, Calif. shRNA plasmids were purchased from Open Biosystems (Thermo Fisher Scientific, Waltham, Mass.), and packaged into inactivated lentivirus particles at University of North Carolina at Chapel Hill Lenti-shRNA core facility. The sequences for the mature sense strands in the hairpins were: shOGT (TRCN0000035064; SEQ ID NO. 72): 5′-GCCCTAAGTTTGAGTCCAAAT-3′, and shOGA (TRCN0000134040): 5′-CCAGAAACTTTCCTTGCTAAT-3′. The TRC Lentiviral eGFP shRNA was used as a Positive Control for transduction (Open Biosystems catalog #RHS4459). Mouse monoclonal O-GlcNAc specific antibodies (clone RL2) were from Thermo Scientific (Waltham, Mass.). Rabbit monoclonal antibodies to GAPDH were from Cell Signaling (Danvers, Mass.). Mouse monoclonal antibodies to β-actin were from OGT-specific antibodies were from Sigma (St. Louis, Mo.). Rabbit polyclonal OGT antibodies were from Abcam (Cambridge, Mass.). Mouse and rabbit anti-sheep horseradish peroxidase-conjugated secondary antibodies were from GE Healthcare (Pittsburgh, Pa.). Control siRNA (sense strand: GCAGUUAUAAUGACUAGAU) and OGT siRNA (sense strand: GCACAAUCCUGAUAAAUUU) with 3′UU overhangs were purchased from Sigma-Aldrich.

Cell Culture and Scratch Wounding.

Untransfected and shRNA transfected HaCaT cells were cultured in normal or high glucose Dulbecco's modified Eagle's medium (DMEM) (5.5 mM or 25 mM glucose, respectively) (13), 1% fetal bovine serum (FBS), 1,000 units penicillin/mL, 100 μg streptomycin/mL. Media were supplemented with the amounts of glucose or inhibitor specified in the figure legends. shRNA-transfected cells were selected using 1 μg puromycin per mL medium. Puromycin-containing media were replaced six hours prior to scratching. Cells were grown for 60 hours (until confluent) before scratch assays were performed. Scratch wounds were performed by making a linear scratch across monolayers of confluent cells in 24-well culture plates followed by one wash with 1×PBS and the addition of fresh culture medium. Pictures were taken on a Nikon TE2000-U spinning disk microscope using a 10× magnification immediately after scratching, and incubated at 37° C. for the amount of time stated in the figure legends before another set of pictures were taken. Wounds were subsequently analyzed using the Tscratch software package (14). Only wounds of the same initial wound-size were evaluated and compared.

Statistical Analyses.

Error bars reflect the standard error of mean (SEM). Student's T-tests were performed as two-sided tests with unequal variance as described in the Tscratch software manual.

Stable Transduction of Keratinocytes with shRNAs.

HaCaT cells were cultured in DMEM, 10% FBS to 50-60% confluency and incubated with 10 μg/mL polybrene and shRNA (shGFP, shOGT, or shOGA) using a multiplicity of infection (MOI) of two for five hours after which the medium was changed to fresh DMEM. The following day, medium containing 1 μg/mL puromycin was added to the cells to select for successfully transduced cells. Cell cultures were passaged 6-8 times under puromycin selection before they were used for experiments.

Quantification of Immunoblot Signals.

Samples were equally loaded on and separated by SDS-PAGE as previously described Immunoblotting was performed according to established protocols and developed by enhanced chemiluminescence (ECL) reaction (Amersham Biosciences). Protein bands from immunoblots were quantified using the GeneSnap software (SynGENE, Frederick, Md.). For RL2 staining, the three most prominent bands were analyzed using GeneSnap software.

siRNA Transfection of Keratinocytes.

siRNA against OGT (3′-GCACAAUCCUGAUAAAUUU-5′) and a scrambled control siRNA (3′-GCAGUUAUAAUGACUAGAU-5′) were synthesized with 3′-UU overhands and were diluted to 20 mM in water (working stock) and each well in a 24-well plate with 40% confluent keratinocytes was transfected using Oligofectamine (Invitrogen) according to the protocol. Briefly, 3 μL Oligofectamine (Invitrogen) was diluted in 12 μL Opti-MEM I (Invitrogen) and incubated for 8 min. In the meantime 3 μL siRNA was mixed with 50 μL Opti-MEM I and this was added to the Oligofectamine dilution and left to form complexes for 20 min. 32 μL Opti-MEM was then added to the mix and added to the cells (in 500 μL high glucose DMEM). After 48 hours the medium was changed to high glucose DMEM and at 60 hours the cells were used for scratch assay. Pictures were taken at the time points described in the figure legends.

Results

Hyperglycemic Conditions Result in Elevated O-GlcNAc Levels in Human Keratinocytes.

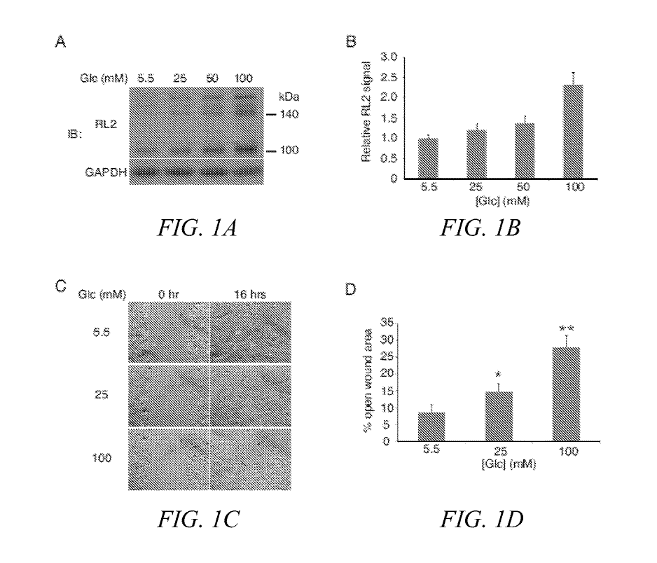

In order to investigate if increased levels of O-GlcNAc modification in diabetic skin may be linked to the high levels of glucose in tissue, these conditions were mimicked in cell culture by growing human keratinocytes (HaCaT) for 48 hours with different amounts of glucose supplemented in the growth media (FIG. 1) Immunoblot of cell lysates shows that increased levels of glucose indeed resulted in more O-GlcNAc modification in keratinocyte lysates, as detected by the O-GlcNAc specific antibody RL2 (FIGS. 1A and 1B). This dose-dependent increase in O-GlcNAcylation emphasizes the link between increased glucose concentrations and the O-GlcNAc modification in keratinocytes

Human Keratinocytes Exhibit Delayed Wound Healing Under Hyperglycemic Conditions.

The present inventors then wanted to test whether hyperglycemic conditions affect the rate of wound closure for human keratinocytes. For this the present inventors utilized the “scratch assay” as an in vitro model for wound healing (FIG. 1C). The assay was performed by pre-incubating HaCaT cells with different amounts of glucose for 48 hours, after which a “wound” was introduced in the confluent layer of cells. Letting the “wound healing” progress for 16 hours shows that elevated levels of glucose in the culture media decreased the rate of wound closure in a dose-dependent manner (FIG. 1D).

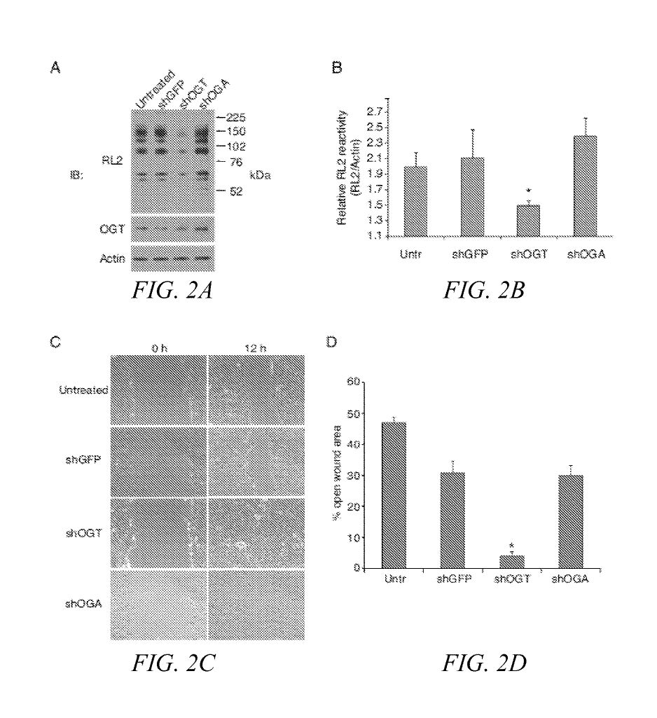

Gene knockdown of key enzymes for the O-GlcNAc pathway by RNA interference affects the rate of wound closure in human keratinocyte culture. The apparent link between delayed wound closure and elevated levels of O-GlcNAc modification in HaCaT cells led us to further investigate the role of the enzymes responsible for the addition and removal of O-GlcNAc protein modification (OGT and OGA, respectively) in more detail. In order to do this, the present inventors stably transducted HaCaT cells with shRNAs against either enzyme and analyzed cell lysates by immunoblot analysis (FIGS. 2A and 2B) Immunoblot analysis of the cell lysates confirmed the impact of RNAi on O-GlcNAc levels, with shOGT displaying significantly reduced levels of O-GlcNAc modification. The shOGA transducted cells displayed levels of O-GlcNAc modification similar to untransducted and shGPF controls (FIG. 2B). Scratch-wounding of shRNA-transducted cells show that knocking down OGT significantly increases the rate of wound closure, while the opposite is true for OGA (FIGS. 2C and 2D). shGFP transducted controls were not significantly different from untransducted cells. Collectively, these data strongly suggest that decreasing the amount of O-GlcNAc (shOGT) accelerates wound healing, whereas suppressing the removal of O-GlcNAc (shOGA) inhibits wound healing in human keratinocytes, thus underlining the link between O-GlcNAc levels and the rate of wound healing.

siRNA Knock-Down of OGT Decreases Keratinocyte O-GlcNAcylation and Accelerates Wound Closure at Hyperglycemic Conditions.

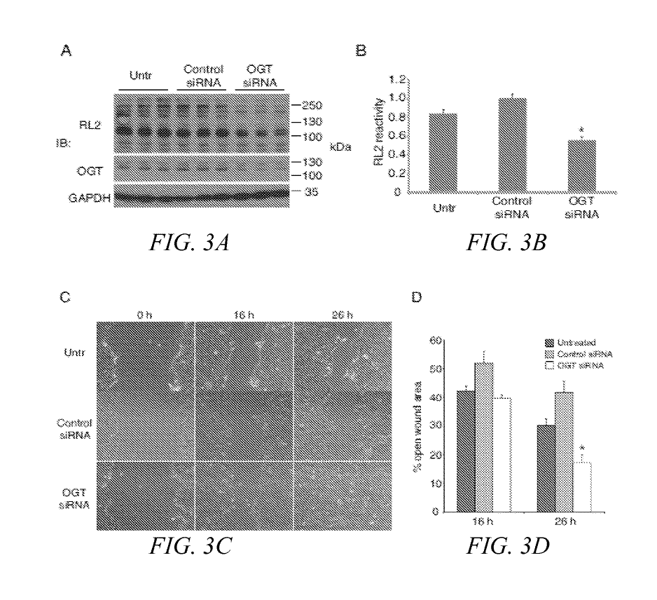

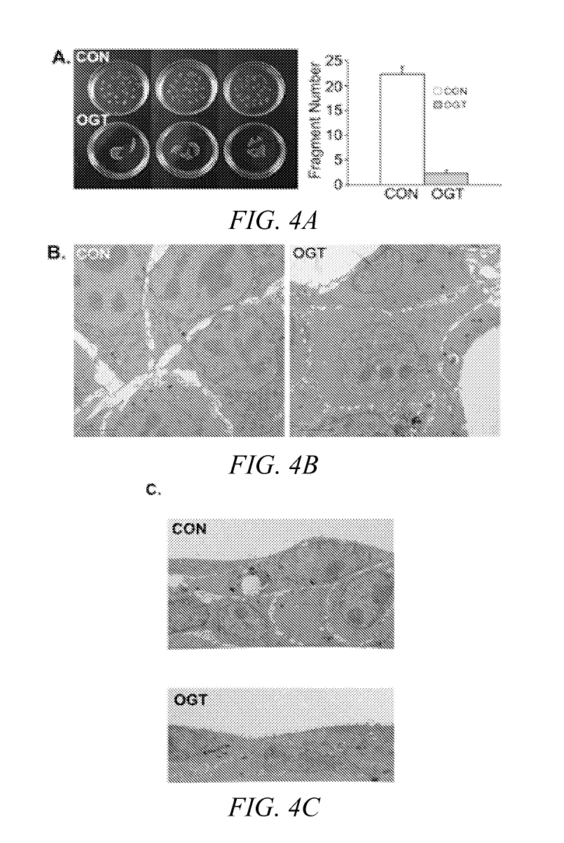

To investigate the potential of using a more therapeutically relevant approach to target the OGT gene expression, the present inventors tested small interfering RNAs (siRNAs) as a means to knock down OGT (FIG. 3). A 19mer siRNA directed against the OGT mRNA sequence was synthesized and HaCaT cells were transfected using an siRNA with a scrambled sequence as a control. Two days post transfection cell lysates were probed for RL2 and OGT immunoreactivity (FIGS. 3A and 3B). The results show that siRNA against OGT results in a marked knock down in both OGT levels and RL2 immunoreactivity as quantified from immunoblots.

Next, siRNA transfected cells were tested in a scratch-wounding assay to examine the effect of this form of OGT RNAi on wound closure in vitro. FIG. 3C shows that wound healing at the 26-hour time point is significantly more progressed with OGT siRNA compared to both control siRNA and untreated cells (FIG. 3D). These results further support that the level of intracellular O-GlcNAc modification in human keratinocytes is linked to wound closure rate and that this may be manipulated using OGT knockdown.

shRNA Knockdown of OGT Decreases Keratinocyte Intracellular Protein O-Glycosylation; Whereas, OGA Knockdown Increases Protein O-Glycosylation.

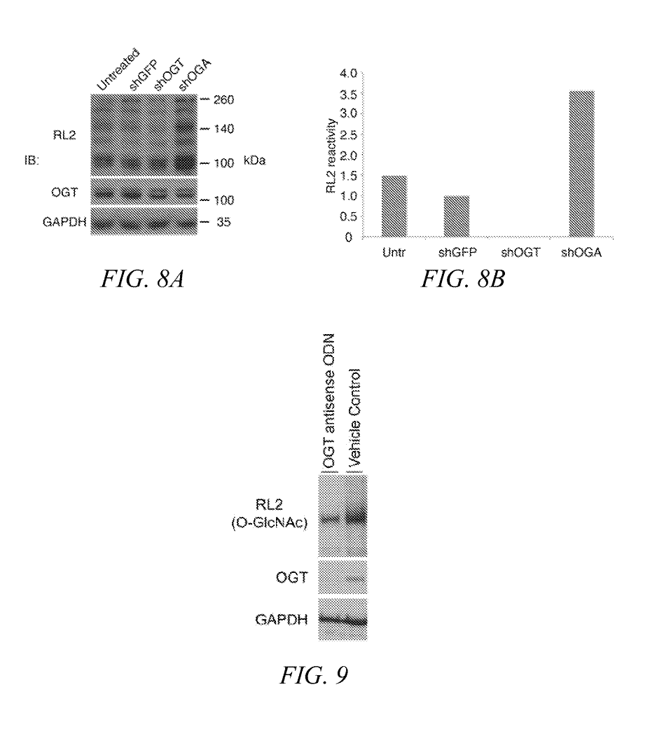

HaCaT cells stably transfected with shRNA targeting GFP (control), OGT, and OGA were grown to confluency. Cell lysates were then analyzed by immunoblotting with antibodies to the (i) O-GlcNAc modification (RL2), (ii) OGT protein, and (iii) GAPDH (loading control), as shown in FIG. 8A and FIG. 8B. As shown, genetic knockdown of OGT decreases O-GlcNAc protein modification; whereas, genetic knockdown of OGA increases O-GlcNAc protein modification.

OGT Antisense Oligodeoxynucleotides Down Regulate OGT Protein Levels and O-GlcNAC Modification in Skin of Test Mice when Applied Topically to a Wound.

Topical application of 10 nM OGT antisense oligodeoxynucleotides in 50 ul Pluronic® F-127 gel to full thickness skin wound of WT mice at t=0 and t=24 hrs post wounding. Perilesional skin of mice treated with OGT antisense ODN or vehicle control was harvested at 48 hrs post wounding. Skin extracts were separated by SDSPAGE and probed by immunoblot with antibodies to the O-GlcNAc modification (RL2), OGT protein, or GAPDH (as a loading control), as shown in FIG. 9.

Discussion

Increasing evidence from the literature suggests that alterations in the hexosamine pathway play a key role in the pathophysiology of diabetes. For example, overexpression of OGT in mice results in a diabetic phenotype (8) and increased levels of O-GlcNAcylation have been observed in cells and tissue from type 2 diabetes patients relative to healthy controls (9,15). Previously, the present inventors had reported that over expression of OGT in keratinocytes (i) increases GlcNAc modification of cellular proteins and (ii) markedly enhances cell-cell adhesion (12). Consistent with these observations, the present inventors observed a dose dependent increase in protein O-glycosylation in human keratinocyte cultures grown in increasing concentrations of glucose. Furthermore, increasing concentrations of glucose and O-GlcNAc protein modification was associated with delayed wound closure in a dose dependent fashion. Significantly, silencing OGT activity with either OGT specific shRNA or siRNA decreases GlcNAc modification of keratinocyte proteins and promotes wound healing in a scratch model assay, even in the presence of elevated glucose concentrations. Collectively, these observations suggest that increased intracellular O-glycosylation, mediated by the enzyme OGT, likely contributes to delayed wound healing in chronic diabetic skin wounds.

The effects of increased OGT activity on promoting cell adhesion and delaying wound healing may in part be due to regulation of keratinocyte cell adhesion components, including desmosomes, adherens junctions, and cytoskeletal elements as the present inventors have previously reported.(12) In this context, the present inventors previously showed that plakoglobin, a component of both adherens junction and desmosome cell-cell adhesion complexes, is post-translationally stabilized by increased O-glycosylation in OGT overexpressing keratinocytes. This increased plakoglobin protein level drove formation of desmosomes and plakoglobin based adherens junctions and markedly enhanced cell-cell adhesion.(12) These observations indicate that in keratinocytes, O-glycosylation functions in part to regulate plakoglobin's post-translational stability and significantly, to regulate keratinocyte cell-cell adhesion. During wound healing, keratinocytes migrate into the wound to promote re-epithelialization. Keratinocytes at the wound margin must down-regulate adhesion to adjacent cells at the trailing margin to permit movement away from the edge and into the wound. By increasing cell-cell adhesion, the present inventors suggest that increased intracellular protein O-glycosylation retards wound healing; whereas, down-regulation of intracellular protein O-glycosylation promotes wound healing.

It is worth noting that O-glycosylation is a ubiquitous intracellular modification. In addition to modifying cell adhesion and structural proteins, transcription factors and regulatory enzymes are also modified by OGT catalyzed addition of GlcNAc to serine and threonine residues. Thus, the effects of OGT activity are likely to be pleiotropic. In addition to its effects on adhesion, altering levels of intracellular protein O-glycosylation may also impact cell proliferation and chemotaxis and it may be the combination of these effects that contribute to the observed delayed wound healing.

Diabetic wounds represent a significant health care burden. The incidence and social and financial cost of treating these wounds is likely to increase as the incidence of diabetes increases due to the rising incidence of obesity and to aging populations. The present inventors have demonstrated that decreasing the global level of O-GlcNAcylation through knockdown of OGT using RNAi accelerates wound healing in a hyperglycemic keratinocyte culture model. Collectively, these data show that locally targeting OGT may prove an effective approach to promote healing in diabetic ulcers. As it has previously been demonstrated that the impaired barrier function in the wounds allows for transfection with oligonucleotides (16), the present inventors suggest that topical administration of siRNAs against OGT may be an effective treatment to promote healing in chronic diabetic skin wounds, as well as wounds in general.

### Example 2

Topical Delivery of OGT Antisense Oligonucleotides to Promote Healing in Diabetic Wounds.

Data of the present inventors indicates that increased intracellular O-glycosylation of proteins catalyzed by the nucleocytoplasmic enzyme O-GlcNAc transferase (OGT) contributes to delayed wound healing in chronic diabetic skin wounds. This studies described in this Example will test if healing of diabetic skin wounds can be accelerated by knockdown of the enzyme OGT in the skin through direct delivery of OGT specific oligonucleotides. The present inventors contemplate that downregulating OGT activity by topical application of OGT antisense oligonucleotides (OGT antisense ODNs) and/or OGT siRNA to skin wound sites will accelerate healing in normal and diabetic wounds.

The studies described in this Example are related to:

I) Determination of whether OGT knockdown in diabetic mouse models can accelerate the rate of wound healing, and

II) Further characterization of mechanism by which OGT mediated intracellular O-glycosylation regulates wound healing.

Topical Delivery of OGT Antisense Oligonucleotides to Promote Healing in Diabetic Wounds

Chronic wounds are a significant source of morbidity affecting 6.5 million patients in the United States and costing approximately $25 billion annually to treat. Patients with diabetes are at increased risk for developing chronic non-healing wounds. A variety of factors likely contribute to the predisposition of diabetic patients to develop chronic wounds including neuropathy, vasculopathy, as well as the underlying endocrine dysfunction that results in elevated glucose levels.

O-glycosylation of serine and threonine residues is a common regulatory post-translational modification analogous to protein phosphorylation; increased intracellular protein O-glycosylation has been observed in diabetic and hyperglycemic states. Two intracellular enzymes, UDP-N-acetylglucosamine-polypeptide β-N-acetylglucosaminyl transferase (OGT) and O-GlcNAc-selective N-acetyl-β-D-glucosaminidase (OGA), mediate addition and removal, respectively, of N-acetylglucosamine (GlcNAc) from intracellular protein substrates. Alterations in O-GlcNAc modification of intracellular proteins is linked to diabetes and the increased levels of protein O-glycosylation observed in diabetic tissues may in part explain some of the observed underlying pathophysiology that contributes to delayed wound healing.

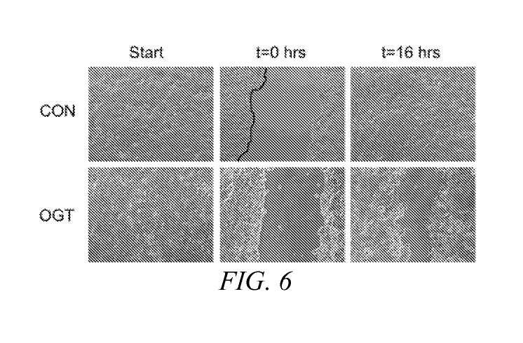

Increased OGT activity in keratinocytes delays wound healing. During wound healing, keratinocytes migrate into the wound to promote re-epithelialization. Keratinocytes at the wound margin must down-regulate adhesion to adjacent cells at the trailing margin to permit movement away from the edge and into the wound. By increasing cell-cell adhesion, the present inventors contemplate that increased intracellular protein O-glycosylation retards wound healing; whereas, down-regulation of intracellular protein O-glycosylation promotes wound healing. In this proposal, the present inventors will further characterize the role of O-glycosylation in wound healing. As a preliminary test, time to wound closure in OGT over-expressing keratinocytes was compared to control keratinocytes using a scratch assay (FIG. 6). Increased OGT mediated O-glycosylation of intracellular proteins resulted in delayed healing. At 16 hrs post wounding, control keratinocytes had completely closed the wound. In contrast, OGT over-expressing keratinocytes had failed to close the wound; less than 50% wound closure was observed in OGT cells at 16 hrs post wounding.

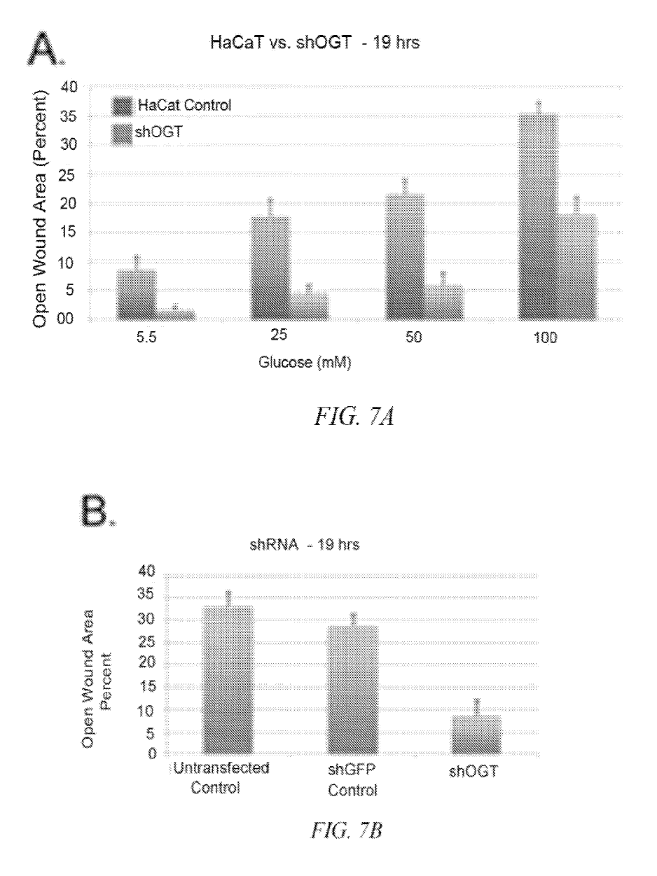

The present inventors have shown that (i) increasing protein O-glycosylation by over-expression of OGT in murine keratinocytes results in elevated protein O-glycosylation and a hyper-adhesive phenotype (FIGS. 2 and (17)) and (ii) human keratinocytes cultured under elevated hyperglycemic conditions display increased levels of O-GlcNAc modification as well as a delay in the rate of wound closure in vitro (FIG. 7). The present inventors have further shown that specific knock-down of OGT by RNA interference (RNAi) reverses this effect significantly (FIGS. 2, 3, 7), thereby opening up the opportunity for OGT-targeted therapeutic intervention in delayed wound healing in diabetic patients. It is contemplated that inhibiting OGT will promote healing of chronic wounds including diabetic wounds. The present inventors propose that topical administration of inhibitory nucleotides (e.g. OGT antisense oligonucleotides or siRNA) will promote wound healing in vivo.

Intracellular O-glycosylation in diabetes. Hyperglycemia, excess glucose, feeds into the glucosamine pathway to provide excess UDP-GlcNAc for OGT to modify intracellular proteins (18). Excess glucose is converted to glucosamine which is ultimately converted to UDP-N-acetylglucosamine (UDP-GlcNAc), the donor substrate for OGT modification of intracellular proteins. Consequently, hyperglycemia is associated with increased O-glycosylation of a variety of proteins (18-22). The increased GlcNAc modification of intracellular proteins observed in hyperglycemic states including diabetes is thought to contribute to some of the pathology associated with diabetes. For example, pancreatic β-cells have high levels of OGT and are sensitive to alterations in intracellular O-GlcNAc modification and over-expression of OGT in muscle and adipose tissue causes diabetes in transgenic mouse models (23).

Genetic association of diabetes and mutations causing increased intracellular protein O-glycosylation. A mutation that results in early termination in the gene encoding O-GlcNAcase (OGA) has been associated with a genetic predisposition to adult onset type II diabetes in a Mexican American population (24). OGA removes GlcNAc from intracellular proteins and the identified OGA mutations result in increased levels of protein O-glycosylation. This observation provides further support for a pathologic role for increased intracellular O-glycosylation in diabetes

Preliminary work suggests that increased intracellular O-glycosylation, mediated by the enzyme OGT, contributes to delayed wound healing in chronic diabetic skin wounds, including:

Overexpression of OGT in keratinocytes (i) increases GlcNAc modification of cellular proteins, (ii) markedly enhances cell-cell adhesion (FIG. 7)(1), and (iii) delays wound closure in a keratinocyte scratch assay model of wound healing (FIG. 6);

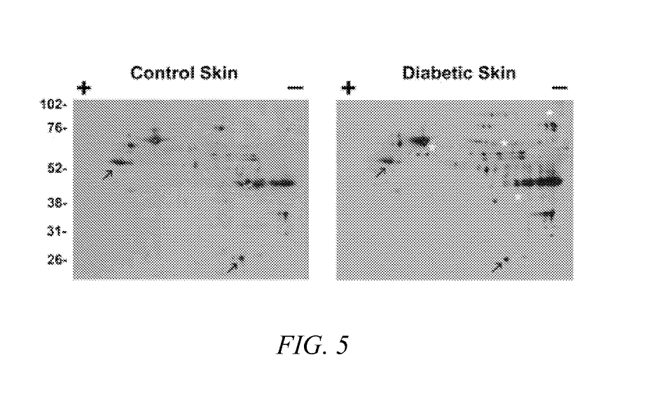

Increased GlcNAc modification of cellular proteins is observed in skin from diabetic mice (FIG. 5);

Increasing concentrations of glucose in human keratinocyte cultures is associated with increased GlcNAc modification of keratinocyte proteins and inhibits wound closure in a dose dependent fashion (FIG. 7); and

Silencing OGT activity with an OGT specific shRNA or siRNA decreases GlcNAc modification of keratinocytes proteins and promotes wound healing in a scratch model assay even in the presence of elevated glucose concentrations (FIGS. 2, 3, 7).

The effects of increased OGT activity on promoting cell adhesion and delaying wound healing may in part be due to regulation of keratinocyte cell adhesion components, including desmosomes, adherens junctions, and cytoskeletal elements as the present inventors have previously reported (17).

Thus, (i) delayed healing of epithelial wounds by increasing intracellular protein O-glycosylation by either increased glucose concentrations, increased OGT activity, or decreased OGA activity and (ii) accelerated wound healing by OGT specific shRNA or siRNA, that decrease protein O-glycosylation, support a role for this pathway in delayed healing of chronic diabetic skin wounds.

The identification of OGT as a regulator of keratinocyte wound healing in diabetes and hyperglycemic states and the demonstration that down-regulating OGT activity promotes wound healing are highly novel and innovative observations. The development of topical antisense OGT ODNs to down-regulate OGT activity in vivo will provide proof of concept that OGT inhibition will accelerate healing of chronic diabetic wounds. Furthermore, topical delivery of antisense ODNs to wounds has been demonstrated in human subjects to down-regulate target genes due to the impaired barrier present in a wound. Thus, topical delivery of OGT antisense ODNs represents a highly novel, practical and viable approach to promote healing and would represent a significant advance in the care of chronic non-healing diabetic wounds. By decreasing the time to healing, topical OGT antisense ODNs are anticipated to decrease both the direct costs of caring for these wounds as well as the indirect costs resulting from loss of productivity in affected individuals. This proposal will test if healing of diabetic skin wounds can be accelerated by knockdown of the enzyme OGT in the skin through direct delivery of OGT specific oligonucleotides. This is a high risk, high impact study that, by proof of concept that OGT inhibition in vivo can promote healing, has the potential to rapidly lead to translational clinical studies of OGT antisense ODNs in patients with chronic diabetic skin wounds.

Approach/Methods:

I. Determine if OGT Knockdown in Diabetic Mouse Models can Accelerate the Rate of Wound Healing

Diabetic mouse models. Initial in vivo studies will focus on the well characterized and readily available streptozoticin (STZ) induced diabetic C57BL/6J mice obtained from Jackson Laboratories (Bar Harbor, Me.); non diabetic C57BL/6J mice will be used as WT controls. Alternatively, using established protocols the present inventors can induce diabetes in 6-8 week old male C57BL/6J mice by intraperitoneal injection of streptozoticin (STZ), 50 mg STZ/kg body weight, qd×5 days. Ten days post injection, blood glucose levels are determined to identify diabetic mice (i.e., non-fasted blood glucose levels >300-400 mg/dl). Additional diabetic mouse models that may be explored include diet-induced obesity models (pre diabetic type 2 diabetes model) and/or db/db mice, both available from Jackson laboratories.

Wound-Healing Experiments in Diabetic Mouse Models.

Mice are anesthetized, shaved, and a full thickness mid-dorsal wound (6 mm diameter circular shaped, 113 mm² area) is created by excising the skin with a 6 mm punch biopsy. Wounded mice either receive no treatment (group 1), vehicle alone (group 2), or topically with OGT antisense-ODN (OGT antisense oligodeoxynucleotides) in vehicle (group 3), or topically with control (OGT sense) ODN in vehicle (group 4). Four full thickness excisional wounds are made on the shaved back on either side of the dorsal midline of diabetic and control mice. 1 μM OGT antisense-ODN in 30% Pluronic F-127 gel (SIGMA) chilled on ice to one wound. 1 μM control OGT sense-ODN to one wound, vehicle alone to one wound, and nothing to the remaining wound. For experiments to examine extent of OGT knockdown and effects on O-GlcNAc modification, wounds and surrounding skin are harvested with 8 mm punch biopsy at various time points (minimum of 8 mice per time point).

Optimizing in vivo dosing. To identify optimal concentrations and dosing regimens, dose response curves of OGT antisense ODNs in vehicle will be employed in the C57BL/6J mice WT background. Concentrations of ODNs in vehicle from 0.01 μM to 10 μM will be explored initially as published studies in vivo have shown this to be effective for knockdown in skin of test animals. 30% Pluronic® F-127 gel (SIGMA) will be the initial vehicle for ODN delivery as this has been demonstrated to be effective for topical delivery of ODNs in vivo; however, additional vehicles may be explored. Time course studies will be performed to determine optimal dosing regimens in vivo. For example, persistence of OGT knockdown after delivery of a single application will be assessed by biopsy of skin of test animals 24 h, 48 h, 4 days and 7 days after application of topical ODNs. Levels of OGT knockdown and the effect of intracellular protein O-GlcNAc modification will be assayed by (i) immunofluorescence and/or immunoperoxidase staining of skin biopsy sections and (ii) immunoblot of skin extracts with antibodies to OGT and O-GlcNAc. Dosing regimens of topical ODNs will be based on the half-life of the knockdown effect. For example, should the knockdown persist for 48 hours, every other day dosing regimens of topical ODNs will be utilized for in vivo wound healing studies.

Assaying wound healing in vivo. The animals will be examined clinically at different time points until complete wound closure is achieved. Wound closure is measured daily until complete healing of non-treated and vehicle control and OGT antisense-ODN treated mice. Wounds from individual mice are digitally photographed and wound areas quantified (Sigma-Scan; Sigma-Aldrich) and standardized and expressed as a percentage of the initial wound size (100%)(9). The mean values (n=8-10 animals per group) are plotted for each time point, ±SEM. A Student's t test is used for comparison of control and OGT antisense-ODN treated groups. Once wound healing is complete, mice will be sacrificed by decapitation following the protocol approved by the Animal Care Committee of UNC-CH. Skin tissue samples will be analyzed for levels of OGT and functionally for protein O-GlcNAc modification by direct immunofluorescence and immunoperoxidase staining of skin biopsies using anti-OGT specific antibodies and O-GlcNAc specific antibodies (clone RL2 or CTD110.), respectively Immunoblot of tissue extracts utilizing antibodies to OGT and RL2 and CTD 110.1 O-GlcNAc specific antibodies is an additional approach to examine the effectiveness of OGT antisense ODN delivery and functional knockdown of OGT.

II. Further Characterize the Mechanism by which OGT Mediated O-Glycosylation Regulates Wound Healing In Vitro and In Vivo.

In addition to adhesion, regulation of cell proliferation and/or chemotaxis could also contribute to delayed wound healing observed in hyperglycemic conditions or OGT over-expressing keratinocytes. This study will further characterize the effects of increased GlcNAc modification of keratinocyte intracellular proteins on (1) cell-cell adhesion, (2) cell proliferation, and (3) chemotaxis. The present inventors contemplate (i) that the effects of OGT activity are likely to be pleitropic, (ii) that levels of intracellular O-glycosylation alter cell proliferation, chemotaxis, and adhesion, and (iii) that the combination of these effects contributes to the observed delayed wound healing.

Rationale: Preliminary data that the present inventors have generated using monolayer scratch assays indicate that increased intracellular O-glycosylation in keratinocytes retards wound healing and that OGT knockdown accelerates wound healing. O-glycosylation is a ubiquitous intracellular modification. In addition to modifying cell adhesion and structural proteins, transcription factors and regulatory enzymes are also modified by OGT catalyzed addition of GlcNAc to serine and threonine residues. In addition to adhesion, O-GlcNAc mediated regulation of cell proliferation and/or chemotaxis could also contribute to delayed wound healing observed in hyperglycemic conditions or OGT over-expressing keratinocytes. This study characterizes the effects of increased GlcNAc modification of keratinocyte intracellular proteins on (1) cell-cell adhesion, (2) cell proliferation, and (3) chemotaxis. The present inventors contemplate (i) that the effects of OGT activity are likely to be pleitropic, (ii) that levels of intracellular O-glycosylation alter cell proliferation, chemotaxis, and adhesion, and (iii) that the combination of these effects contributes to the observed delayed wound healing.

In vitro assays. Using primary human keratinocytes as well as permanently transfected immortalized keratinocyte cell lines, the present inventors will examine the effects of altered intracellular O-glycosylation. O-glycosylation levels are manipulated by transfection of human OGT in cell lines, by shRNA knockdown of endogenous OGT and of endogenous OGA, by exposure of cells to increased concentrations of extracellular glucose, and by utilizing the OGA inhibitor PUGNAc. Using these tools to manipulate the levels of intracellular O-glycosylation, the present inventors will assay for alterations in (1) cell proliferation, (2) cell migration, (3) cell-cell adhesion, and (4) wound closure, as outlined below.

The present inventors will determine whether levels of intracellular O-glycosylation affect cell proliferation. Assays for cell proliferation will include BrDu incorporation and ³H-thymidine metabolic radio labeling experiments to facilitate quantitative analysis. Based on preliminary results in which human OGT was transiently transfected into the immortalized keratinocyte A431 squamous cell carcinoma, the present inventors predict that increased OGT activity and intracellular O-glycosylation will decrease proliferation; whereas, decreased O-glycosylation will increase proliferation.

The present inventors will determine whether levels of intracellular O-glycosylation affect cell chemotaxis. Modified Boyden Chamber assays will be employed to assay for altered chemotaxis to a variety of agents known to stimulate keratinocyte motility including fetal bovine serum, EGF, PDGF and GPCR agonists. The present inventors predict that increased OGT activity and intracellular O-glycosylation will decrease chemotaxis.

The present inventors will determine whether the levels of intracellular O-glycosylation affect cell-cell adhesion. Dispase assay will be used to directly measure cell-cell adhesion and the effects of junction protein components will be analyzed by immunoblot and IF analysis using antibodies to adherens junction and desmosome components to determine effects of OGT on junction protein levels and cellular localization as previously described (17). The present inventors predict that increased OGT activity and intracellular O-glycosylation will enhance cell-cell adhesion.

The present inventors will examiner all three effects simultaneously in models of wound healing (i. scratch and ii. organotypic). The present inventors will utilize both keratinocyte monolayer scratch assay and skin equivalent organotypic cultures to investigate the effects of intracellular O-glycosylation on wound healing. Permanently transfected OGT keratinocytes will be used to drive increased intracellular O-glycosylation in these model systems. Additionally, shRNA knockdown of either OGT or O-GlcNAcase in keratinocytes in monolayer cultures or in keratinocytes used to construct skin equivalents will enable us to examine alterations in endogenous OGT and OGA activity. OGT shRNA and OGA shRNA will be utilized to investigate the effects of decreased and increased intracellular O-glycosylation, respectively, on the wound healing process. The present inventors will assay for alterations in (1) cell-cell adhesion, (2) cell migration, (3) cell proliferation, and (4) wound closure. The present inventors predict that increased OGT activity and intracellular O-glycosylation will decrease wound healing.

In Vitro Assays:

Manipulating OGT Activity.

Several approaches will be utilized to modulate OGT activity within cell and tissue model systems. The present inventors have generated permanently transfected murine and human keratinocyte cell lines that drive over-expression of cloned murine and human OGT, respectively. Additionally, the present inventors have shRNA constructs to both OGT and OGA that the present inventors have shown to be effective at significantly reducing endogenous levels of OGT and OGA mRNA, protein, and activity. Modulation of extracellular glucose concentrations has also been shown to alter intracellular protein GlcNAc modification (2). Finally, inhibitors of OGA, specifically PUGNAC can be employed to increase intracellular O-glycosylation by inhibiting the enzyme that catalyzes the removal of GlcNAC from serine and threonine residues.

Assays for O-Glycosylation.

A number of assays will be employed to detect increased OGT-mediated GlcNAc modification of intracellular proteins. Detection of GlcNAc modified protein is by (i) immunoblot with the commercially available GlcNAc specific monoclonal antibodies RL2 (26-29) (Affinity Bioreagents) or CTD110.6 (Covance), (ii) specific incorporation of radiolabel into protein substrates by culturing cells in ³H-GlcNAC, or (iii) by galactosyltransferase radio labeling; β1,4-galactotransferase catalyzes the addition of galactose from the donor UDP-galactose to the OH-4 of N-acetylglucosamine and therefore can be utilized to specifically incorporate ³H-galactose into GlcNAc modified cellular proteins (30). The present inventors have used each of these approaches to demonstrate plakoglobin O-glycosylation (17).

Assays for Cell Proliferation.

Cells are incubated for 3 h with 10 μM of the thymidine analog 5-bromo-2′-deoxyuridine (BrdU) (which is preferentially incorporated into newly replicated DNA) and fixed in 4% paraformaldehyde containing 5% sucrose (pH 7.0) for 20 min at room temperature. Nuclei incorporating BrdU are detected with an anti-BrdU antibody and counted using a Zeiss fluorescence microscope. At least 500 cells are scored per point, including at least 5 different randomly chosen fields. Alternatively, proliferating keratinocytes are labeled with 3H-thymidine; the level of ³H signal incorporated into a defined number of cells for each experimental condition provides a direct quantitative measure of cell proliferation. Other approaches to measuring proliferation rate include staining with crystal violet or MTT and counting the number of positively stained cells.

Assay for cell-cell adhesion will be via the dispase-based dissociation assay as described (17, 31, 32). Briefly, cells grown to confluence in triplicate on 12 or 24 well tissue culture plates are washed twice with PBS, incubated in 0.25 or 0.05 ml of dispase II (2.4 U/ml; Roche Diagnostics GmbH), respectively at 37° C. for 1 h, rocked back and forth 10 times on a ClayAdams nutator, and the number of fragments counted.

Assay for Chemotaxis.

Chemotaxis is measured in a modified Boyden chamber, using polycarbonate filters (25 by 80 mm, 12 μM pore size). Chemoattractants are added to the lower chamber, and cells added to the upper chamber at 5×10⁴ cells/well. At specified time points (0-12 hrs), non-migratory cells on the upper membrane surface are mechanically removed and the cells that traverse and spread on the lower surface of the filter fixed and stained with Diff-Quik (Fisher Scientific, Pittsburgh, Pa.). The migrated cells are counted with a microscope and a 10× objective. For each data point, four random fields are each counted twice, and the average+/−standard deviation (SD) of three individual wells determined. Effects on chemotaxis of OGT mediated intracellular O-glycosylation may be global or pathway specific. To distinguish between these possibilities, assays will be performed to various chemotactic agents (growth factors such as EGF and PDGF, serum, and GPCR agonists).

In Vivo Assays:

In addition to altering cell adhesion, OGT may affect proliferation. Keratinocyte proliferation will be assayed in vivo by immunohistochemical staining of skin biopsies for Ki67, nuclear proliferating antigen (33) to determine effects of OGT knockdown on in vivo keratinocyte proliferation post wounding.

During wound healing, there are progressive changes in the inflammatory infiltrate, including early infiltration by neutrophils and then macrophages. Inflammatory changes are thought to impair early wound healing, thus, the present inventors will additionally analyze tissue samples for the effect of OGT antisense ODNs for effects on the inflammatory infiltrate in order to determine effects of OGT knockdown on wound inflammation in vivo.

Neutrophil and macrophage infiltration will be assessed by skin biopsy of the wound on d1 and d3 post wounding. Sections will be stained with hematoxylin and eosin and neutrophils quantified by counting cells/high power field. Additionally, staining for myeloperoxidase will also be used as an additional means to quantify neutrophil infiltrate into the wound. Macrophages will be quantified by staining skin biopsy samples.

### REFERENCES

1. Sen, C. K., Gordillo, G. M., Roy, S., Kirsner, R., Lambert, L., Hunt, T. K., Gottrup, F., Gurtner, G. C., and Longaker, M. T. (2009) Human skin wounds: a major and snowballing threat to public health and the economy. Wound Repair Regen 17, 763-771
2. Hart, G. W., Housley, M. P., and Slawson, C. (2007) Cycling of O-linked beta-N-acetylglucosamine on nucleocytoplasmic proteins. Nature 446, 1017-1022
3. Konrad, R. J., Janowski, K. M., and Kudlow, J. E. (2000) Glucose and streptozotocin stimulate p135 O-glycosylation in pancreatic islets. Biochem Biophys Res Commun 267, 26-32
4. Liu, K., Paterson, A. J., Chin, E., and Kudlow, J. E. (2000) Glucose stimulates protein modification by O-linked GlcNAc in pancreatic beta cells: linkage of O-linked GlcNAc to beta cell death. Proc Natl Acad Sci USA 97, 2820-2825
5. Dentin, R., Hedrick, S., Xie, J., Yates, J., 3rd, and Montminy, M. (2008) Hepatic glucose sensing via the CREB coactivator CRTC2. Science 319, 1402-1405
6. Konrad, R. J., Tolar, J. F., Hale, J. E., Knierman, M. D., Becker, G. W., and Kudlow, J. E. (2001) Purification of the O-glycosylated protein p135 and identification as O-GlcNAc transferase. Biochem Biophys Res Commun 288, 1136-1140
7. Konrad, R. J., Mikolaenko, I., Tolar, J. F., Liu, K., and Kudlow, J. E. (2001) The potential mechanism of the diabetogenic action of streptozotocin: inhibition of pancreatic beta-cell O-GlcNAc-selective N-acetyl-beta-D-glucosaminidase. Biochem J 356, 31-41
8. McClain, D. A., Lubas, W. A., Cooksey, R. C., Hazel, M., Parker, G. J., Love, D. C., and Hanover, J. A. (2002) Altered glycan-dependent signaling induces insulin resistance and hyperleptinemia. Proc Natl Acad Sci USA 99, 10695-10699
9. Park, K., Saudek, C. D., and Hart, G. W. (2010) Increased expression of beta-N-acetylglucosaminidase in erythrocytes from individuals with pre-diabetes and diabetes. Diabetes 59, 1845-1850
10. Lehman, D. M., Fu, D. J., Freeman, A. B., Hunt, K. J., Leach, R. J., Johnson-Pais, T., Hamlington, J., Dyer, T. D., Arya, R., Abboud, H., Goring, H. H., Duggirala, R., Blangero, J., Konrad, R. J., and Stern, M. P. (2005) A single nucleotide polymorphism in MGEAS encoding O-GlcNAc-selective N-acetyl-beta-D glucosaminidase is associated with type 2 diabetes in Mexican Americans. Diabetes 54, 1214-1221
11. Gurtner, G. C., Werner, S., Barrandon, Y., and Longaker, M. T. (2008) Wound repair and regeneration. Nature 453, 314-321
12. Hu, P., Berkowitz, P., Madden, V. J., and Rubenstein, D. S. (2006) Stabilization of plakoglobin and enhanced keratinocyte cell-cell adhesion by intracellular O-glycosylation. J Biol Chem 281, 12786-12791
13. Clark, R. J., McDonough, P. M., Swanson, E., Trost, S. U., Suzuki, M., Fukuda, M., and Dillmann, W. H. (2003) Diabetes and the accompanying hyperglycemia impairs cardiomyocyte calcium cycling through increased nuclear O-GlcNAcylation. J Biol Chem
14. Geback, T., Schulz, M. M., Koumoutsakos, P., and Detmar, M. (2009) TScratch: a novel and simple software tool for automated analysis of monolayer wound healing assays. Biotechniques 46, 265-274
15. Jensen, R. V., Zachara, N. E., Nielsen, P. H., Kimose, H. H., Kristiansen, S. B., and Botker, H. E. (2013) Impact of O-GlcNAc on cardioprotection by remote ischaemic preconditioning in non-diabetic and diabetic patients. Cardiovasc Res 97, 369-378
16. Wang, C. M., Lincoln, J., Cook, J. E., and Becker, D. L. (2007) Abnormal connexin expression underlies delayed wound healing in diabetic skin. Diabetes 56, 2809-2817
17. Hu, P., Berkowitz, P., Madden, V. J., and Rubenstein, D. S. (2006) Stabilization of plakoglobin and enhanced keratinocyte cell-cell adhesion by intracellular O-glycosylation. J Biol Chem 281, 12786-12791
18. Konrad, R. J., Janowski, K. M., and Kudlow, J. E. (2000) Glucose and streptozotocin stimulate p135 O-glycosylation in pancreatic islets. Biochem Biophys Res Commun 267, 26-32
19. Liu, K., Paterson, A. J., Chin, E., and Kudlow, J. E. (2000) Glucose stimulates protein modification by O-linked GlcNAc in pancreatic beta cells: linkage of O-linked GlcNAc to beta cell death. Proc Natl Acad Sci USA 97, 2820-2825
20. Dentin, R., Hedrick, S., Xie, J., Yates, J., 3rd, and Montminy, M. (2008) Hepatic glucose sensing via the CREB coactivator CRTC2. Science 319, 1402-1405
21. Konrad, R. J., Tolar, J. F., Hale, J. E., Knierman, M. D., Becker, G. W., and Kudlow, J. E. (2001) Purification of the O-glycosylated protein p135 and identification as O-GlcNAc transferase. Biochemical & Biophysical Research Communications. 288, 1136-1140
22. Konrad, R. J., Mikolaenko, I., Tolar, J. F., Liu, K., and Kudlow, J. E. (2001) The potential mechanism of the diabetogenic action of streptozotocin: inhibition of pancreatic beta-cell O-GlcNAc-selective N-acetyl-beta-D-glucosaminidase. Biochem J 356, 31-41
23. McClain, D. A., Lubas, W. A., Cooksey, R. C., Hazel, M., Parker, G. J., Love, D. C., and Hanover, J. A. (2002) Altered glycan-dependent signaling induces insulin resistance and hyperleptinemia. Proc Natl Acad Sci USA 99, 10695-10699
24. Lehman, D. M., Fu, D. J., Freeman, A. B., Hunt, K. J., Leach, R. J., Johnson-Pais, T., Hamlington, J., Dyer, T. D., Arya, R., Abboud, H., Goring, H. H., Duggirala, R., Blangero, J., Konrad, R. J., and Stern, M. P. (2005) A single nucleotide polymorphism in MGEAS encoding O-GlcNAc-selective N-acetyl-beta-D glucosaminidase is associated with type 2 diabetes in Mexican Americans. Diabetes 54, 1214-1221
25. Kung, H. N., Yang, M. J., Chang, C. F., Chau, Y. P., and Lu, K. S. (2008) In vitro and in vivo wound healing-promoting activities of beta-lapachone. Am J Physiol Cell Physiol 295, C931-943
26. Snow, C. M., Senior, A., and Gerace, L. (1987) Monoclonal antibodies identify a group of nuclear pore complex glycoproteins. Journal of Cell Biology 104, 1143-1156
27. Starr, C. M., and Hanover, J. A. (1990) Glycosylation of nuclear pore protein p62. Reticulocyte lysate catalyzes O-linked N-acetylglucosamine addition in vitro. Journal of Biological Chemistry 265, 6868-6873
28. Holt, G. D., Snow, C. M., Senior, A., Haltiwanger, R. S., Gerace, L., and Hart, G. W. (1987) Nuclear pore complex glycoproteins contain cytoplasmically disposed O-linked N-acetylglucosamine. Journal of Cell Biology 104, 1157-1164
29. Sayeski, P. P., and Kudlow, J. E. (1996) Glucose metabolism to glucosamine is necessary for glucose stimulation of transforming growth factor-alpha gene transcription. Journal of Biological Chemistry 271, 15237-15243
30. Roquemore, E. P., Chou, T. Y., and Hart, G. W. (1994) Detection of O-linked N-acetylglucosamine (O-GlcNAc) on cytoplasmic and nuclear proteins. Methods in Enzymology 230, 443-460
31. Huen, A. C., Park, J. K., Godsel, L. M., Chen, X., Bannon, L. J., Amargo, E. V., Hudson, T. Y., Mongiu, A. K., Leigh, I. M., Kelsell, D. P., Gumbiner, B. M., and Green, K. J. (2002) Intermediate filament-membrane attachments function synergistically with actin-dependent contacts to regulate intercellular adhesive strength. J Cell Biol 159, 1005-1017
32. Ishii, K., Harada, R., Matsuo, I., Shirakata, Y., Hashimoto, K., and Amagai, M. (2005) In vitro keratinocyte dissociation assay for evaluation of the pathogenicity of anti-desmoglein 3 IgG autoantibodies in pemphigus vulgaris. J Invest Dermatol 124, 939-946
33. Usui, M. L., Mansbridge, J. N., Carter, W. G., Fujita, M., and Olerud, J. E. (2008) Keratinocyte migration, proliferation, and differentiation in chronic ulcers from patients with diabetes and normal wounds. J Histochem Cytochem 56, 687-696

All publications, patents, and patent applications mentioned in this specification are herein incorporated by reference to the same extent as if each individual publication, patent, or patent application was specifically and individually indicated to be incorporated by reference.

It will be understood that various details of the presently disclosed subject matter can be changed without departing from the scope of the subject matter disclosed herein. Furthermore, the foregoing description is for the purpose of illustration only, and not for the purpose of limitation.

### SEQUENCE LISTING FREE TEXT

SEQ ID NO: 1 is an OGT sense DNA/nucleotide sequence—coding (3141 nt).

SEQ ID NO: 2 is an OGT polypeptide sequence (1046 aa).

SEQ ID NO: 3 is an OGT antiparallel sequence/OGT antisense DNA sequence.

SEQ ID NOs: 4-11 are OGT antisense olidodesoxynucleotides, wherein SEQ. ID. NO. 4, 5, and 6 are coding regions, SEQ. ID. NO. 7 and 8 identify 3′UTR region(s), and SEQ. ID. NO. 9, 10 and 11 identify introns.

SEQ ID NOs: 12-168 identify OGT antisense oligodeoxynucleotides (coding region).

SEQ ID NOs: 169-171 are OGT siRNA.

SEQ ID NO: 172 is OGT shRNA.

SEQ ID NO: 173 is OGT sense DNA/nucleotide sequence, the full sequence, including regulatory regions, introns, and with coding sequence (49836 nt).

1-3. (canceled)

4. A pharmaceutical composition comprising
(i) an antisense OGT polynucleotide, wherein the polynucleotide is 18-30 nucleotides in length and has a sequence that hybridizes to OGT mRNA; and
(ii) a pharmaceutically acceptable carrier.

5. The composition of claim 4, wherein the polynucleotide has
a sequence set forth in SEQ ID NO: 3, or
a sequence complementary to an 18-30 nucleotide sequence set forth in SEQ ID NO: 1 or SEQ ID NO: 173.

6-9. (canceled)

10. The composition of claim 4, wherein the polynucleotide comprises a sequence selected from any one of SEQ ID NOs: 4-168.

11-13. (canceled)

14. The composition of claim 4, wherein the pharmaceutically-acceptable carrier is selected from the group consisting of a gel, an alcohol, a polyoxyethylene-polyoxypropylene copolymer, Pluronic® F-127, an alginate or hydrogel, a cellulose-based carrier, hydroxymethyl cellulose, hydroxyethyl cellulose, carboxymethyl cellulose, hydroxypropylmethyl cellulose, and mixtures thereof.

15-20. (canceled)

21. The composition of claim 4, wherein the composition is prepared for topical application or local injection.

22. (canceled)

23. The composition of claim 4, wherein the composition is provided in a wound dressing or a colloidal gel dressing.

24-37. (canceled)

38. The composition of claim 4, and further comprising a second wound healing agent and/or a second anti-OGT agent.

39-40. (canceled)

41. A method of treating a subject having a wound, comprising administering an anti-OGT agent to the subject, wherein the anti-OGT agent is chosen from
(i) an antisense OGT polynucleotide, wherein the polynucleotide is 18-30 nucleotides in length and has a sequence that hybridizes to OGT mRNA;
(ii) a double stranded RNA molecule that inhibits expression of OGT; and
(iii) a short hairpin RNA molecule that inhibits expression of OGT.

42. (canceled)

43. The method of claim 41, wherein the polynucleotide has the sequence set forth in SEQ ID NO: 3, or a sequence complementary to an 18-30 nucleotide sequence set forth in SEQ ID NO: 1 or SEQ ID NO: 173.

44-45. (canceled)

46. The method of claim 41, wherein the antisense OGT polynucleotide comprises a sequence selected from any one of the sequences of SEQ ID NOs: 4-168.

47-51. (canceled)

52. The method of claim 41, wherein the isolated double-stranded RNA molecule includes a first strand comprising a sequence selected from SEQ ID NOS: 169-171, and including about 11 to 27 nucleotides.

53. The method of claim 41, wherein the small hairpin RNA comprises the sequence of SEQ ID NO: 172.

54-64. (canceled)

65. The method of claim 41, wherein the composition is administered from a wound dressing or a colloidal gel dressing.

66-69. (canceled)

70. The method of claim 41, wherein the anti-OGT agent is administered topically or by local injection.

71-73. (canceled)

74. The method of claim 41, wherein the wound is not healing at an expected rate, delayed, difficult to heal, or chronic.

75. The method of claim 41, wherein the wound is characterized at least in part by increased expression of OGT.

76-78. (canceled)

79. The method of claim 41, wherein the subject is diabetic.

80-81. (canceled)

82. The method of claim 41, wherein the subject has higher than normal blood glucose levels.

83. The method of claim 41, the subject has insulin-resistant receptors.

84. The method of claim 41, further comprising the step of administering a second wound healing agent and/or a second anti-OGT agent.

85. (canceled)
